(19) United States
(12) Patent Application Publication
(10) Pub. No.: US 2024/0350682 A1
(43) Pub. Date: Oct. 24, 2024

(54) STEAP2-TARGETED COMPOUNDS AND USE THEREOF

(71) Applicants: FUSION PHARMACEUTICALS INC., Hamilton (CA); ASTRAZENECA UK LIMITED, Cambridge (GB)

(72) Inventors: David Rodriguez Caicedo, Ancaster (CA); William Leslie Turnbull, Burlington (CA); Saleemulla Mahammad, Ancaster (CA); Dewald van Dyk, Wilmington, DE (US); Darlene Anne Monlish, Wilmington, DE (US); Vanessa Marie Muniz-Medina, Wilmington, DE (US); Nathanael David Sallada, Wilmington, DE (US); Sao Fong Cheung, Wilmington, DE (US); Jeong Min Han, Wilmington, DE (US); Frank Irvine Comer, Wilmington, DE (US); Asurayya Worrede, Wilmington, DE (US); Chien-ying Chang, Wilmington, DE (US)

(21) Appl. No.: 18/633,413

(22) Filed: Apr. 11, 2024

Related U.S. Application Data
(60) Provisional application No. 63/495,561, filed on Apr. 12, 2023.

(51) Int. Cl. A61K 51/10; A61P 35/00

(52) U.S. Cl. CPC A61K 51/1075; A61K 51/1093; A61P 35/00

(57) ABSTRACT

The present disclosure provides compounds or pharmaceutically acceptable salts thereof, e.g., radioimmunoconjugates including a chelating moiety or a metal complex thereof, a linker, and an antibody or antigen-binding fragment thereof targeting STEAP2. The present disclosure also provides pharmaceutical compositions of such compounds or pharmaceutically acceptable salts thereof and methods of treatment for conditions, e.g., cancer, using such compounds, pharmaceutically acceptable salts thereof, or pharmaceutical compositions.

### RELATED APPLICATIONS

This application claims priority to U.S. Provisional Application Ser. No. 63/495,561, filed Apr. 12, 2023. The content of this application is incorporated by reference in its entirety.

### SEQUENCE LISTING

This application contains a Sequence Listing named 16266_6000-00000_SL.xml, which was created Mar. 14, 2024, and is 39,334 bytes in size. The Sequence Listing has been filed electronically in XML format and is hereby incorporated by reference in its entirety.

### DESCRIPTION

### Technical Field

This disclosure relates to compounds or pharmaceutically acceptable salts thereof (e.g., radioimmunoconjugates) that target STEAP2, pharmaceutical compositions thereof, and methods of treating cancer using such compounds, pharmaceutically acceptable salts thereof, or pharmaceutical compositions.

### BACKGROUND

Prostate cancer is a multifactorial disease and the second most common cancer diagnosed in men worldwide. It is one of the leading causes of death in men with one in eight men being diagnosed in the UK and one in five/six in the USA. The six-transmembrane epithelial antigen of prostate-2 (STEAP2) protein plays an important role in prostate tumorigenesis, cell proliferation, and metastasis. The STEAP2 protein is expressed at very low levels in normal cells, whereas in prostate cancer cells STEAP2 protein is highly expressed. Elevated expression of STEAP2 in prostate cancer cells is correlated with cancer progression, metastasis, and poor survival outcomes. Studies suggest that STEAP2 expression is independent of androgen receptor and prostate-specific membrane antigen (PSMA) expression, two receptors commonly targeted therapeutically, which often develop resistance to targeted treatments. STEAP2 is expressed in both primary and metastatic prostate cancer samples. Increasing evidence has implicated STEAP2 as potential therapeutic target and biomarker for prostate cancer.

Similar to human patients with prostate cancer, STEAP2 overexpression contributes to disease progression and poor survival outcomes in preclinical mouse models with STEAP2-positive xenografted tumors.

The fact that normal tissues express negligible levels and prostate cancer cells show elevated expression of STEAP2 makes it an ideal target for cancer therapy.

Current treatment options for prostate cancer include surgery, hormonal therapy, chemotherapy, radiation therapy, and immunotherapy. These therapies may lead to nonspecific effects (xerostomia, effect on kidney functions) or may not result in long-term therapeutic efficacy. Additionally, treatments targeting two common receptors (Androgen receptor and PSMA) often lead to development of treatment resistant and aggressive forms of prostate cancer. Treatment of metastatic prostate cancers remains to be a long-standing challenge in the field.

Thus, there remains a need for improved therapeutics (e.g., cancer therapeutics) that can target STEAP2 as target-specific, curative therapies without the above drawbacks.

### SUMMARY

The present disclosure relates to compounds or pharmaceutically acceptable salts thereof (e.g., radioimmunoconjugates) that target STEAP2, pharmaceutical compositions thereof, and methods of treating cancer using such compounds, pharmaceutically acceptable salts thereof, or pharmaceutical compositions. Without being bound by theory, unlike naked antibodies, radioimmunoconjugates do not need to block the receptor function to have therapeutic efficacy, instead they emit radioactive particles (e.g., alpha emitters) that target the surrounding tumor cells within the limited range thus preventing any off target associated toxicity. STEAP2-targeted radioimmunoconjugates used for STEAP2 overexpressing cancers utilize the ability of STEAP2 complex to undergo antibody triggered internalization to deliver the targeted radionuclides inside the cancer cells specifically. Antibody monovalent binding to either of the receptors on normal healthy tissues/cells may not lead to internalization.

In certain embodiments, provided compounds (e.g., radioimmunoconjugates) exhibit an increased excretion rate (e.g., after being administered to a mammal) compared to currently known radiotherapeutics, while still maintaining therapeutic efficacy. In some embodiments, a faster excretion may limit off-target toxicities by limiting the amount of time that the compound stays in a subject. Thus, in some embodiments, provided compounds exhibit reduced off-target toxicities.

In one aspect, provided are compounds comprising the following structure, or pharmaceutically acceptable salts thereof:

A-L¹-(L²)ₙ-B Formula I

wherein
A is a chelating moiety or a metal complex thereof,
B is an antibody or antigen-binding fragment thereof,
L¹ is a bond, C═O, C═S, optionally substituted C₁-C₆ alkyl, optionally substituted C₁-C₆ heteroalkyl, optionally substituted aryl, or optionally substituted heteroaryl;
n is an integer between 1 and 5 (inclusive); and
L² each independently has the structure of Formula II:

—X¹-L³-Z¹— Formula II
wherein
X¹ is —C(O)NR¹—*, —NR¹C(O)—*, —C(S)NR¹—*, —NR¹C(S)—*, —OC(O)NR¹—*, —NR¹C(O)O—*, —NR¹C(O)NR¹—, —CH₂-Ph-C(O)NR¹—*, —NR¹C(O)-Ph-CH₂—*, —CH₂-Ph-NH—C(S)NR¹—*, —NR¹C(S)—NH-Ph-CH₂—*, —O—, or —NR¹—, wherein “*” indicates the attachment point to L³, and R¹ is hydrogen, optionally substituted C₁-C₆ alkyl, optionally substituted C₁-C₆ heteroalkyl, optionally substituted aryl, or optionally substituted heteroaryl;
L³ is optionally substituted C₁-C₅₀alkyl or optionally substituted C₁-C₅₀ heteroalkyl; and
Z¹ is —CH₂— #, —C(O)— #, —C(S)— #, —OC(O)— #, —C(O)O— #, —NR²C(O)— #, —C(O)NR²— #, or —NR²— #, wherein “#” indicates the attachment point to B, and R² is hydrogen, optionally substituted C₁-C₆ alkyl, optionally substituted C₁-C₆ heteroalkyl, optionally substituted aryl, or optionally substituted heteroaryl,
wherein the antibody or antigen-binding fragment thereof binds to six-transmembrane epithelial antigen of prostate-2 (STEAP2) and comprises:
a) HCDR1 comprising, having or consisting of the sequence of SEQ ID NO:1, HCDR2 comprising, having or consisting of the sequence of SEQ ID NO:2 and HCDR3 comprising, having or consisting of the sequence of SEQ ID NO:3 and LCDR1 comprising, having or consisting of the sequence of SEQ ID NO:6, LCDR2 comprising, having or consisting of the sequence of SEQ ID NO:7 and LCDR3 comprising, having or consisting of the sequence of SEQ ID NO:8;
(b) HCDR1 comprising, having or consisting of the sequence of SEQ ID NO:11, HCDR2 comprising, having or consisting of the sequence of SEQ ID NO:12 and HCDR3 comprising, having or consisting of the sequence of SEQ ID NO:13 and LCDR1 comprising, having or consisting of the sequence of SEQ ID NO:16, LCDR2 comprising, having or consisting of the sequence of SEQ ID NO:17 and LCDR3 comprising, having or consisting of the sequence of SEQ ID NO:18;
(c) HCDR1 comprising, having or consisting of the sequence of SEQ ID NO:21, HCDR2 comprising, having or consisting of the sequence of SEQ ID NO:22 and HCDR3 comprising, having or consisting of the sequence of SEQ ID NO:23 and LCDR1 comprising, having or consisting of the sequence of SEQ ID NO:25, LCDR2 comprising, having or consisting of the sequence of SEQ ID NO:26 and LCDR3 comprising, having or consisting of the sequence of SEQ ID NO:27; or
(d) HCDR1 comprising, having or consisting of the sequence of SEQ ID NO:31, HCDR2 comprising, having or consisting of the sequence of SEQ ID NO:32 and HCDR3 comprising, having or consisting of the sequence of SEQ ID NO:33 and LCDR1 comprising, having or consisting of the sequence of SEQ ID NO:35, LCDR2 comprising, having or consisting of the sequence of SEQ ID NO:36 and LCDR3 comprising, having or consisting of the sequence of SEQ ID NO:37, or a functional variant of an antibody or antigen-binding fragment of any one of (a)-(d).

In another aspect, provided are compounds comprising the following structure, or pharmaceutically acceptable salts thereof:

A-L¹-(L²)ₙ-B Formula I

wherein
A is a chelating moiety or a metal complex thereof,
B is an antibody or antigen-binding fragment thereof,
L¹ is a bond, C═O, C═S, optionally substituted C₁-C₆ alkyl, optionally substituted C₁-C₆ heteroalkyl, optionally substituted aryl, or optionally substituted heteroaryl;
n is an integer between 1 and 5 (inclusive); and
L² each independently has the structure of Formula II:

—X¹-L³-Z¹— Formula II
wherein
X¹ is —C(O)NR¹—*, —NR¹C(O)—*, —C(S)NR¹—*, —NR¹C(S)—*, —OC(O)NR¹—*, —NR¹C(O)O—*, —NR¹C(O)NR¹—, —CH₂-Ph-C(O)NR¹—*, —NR¹C(O)-Ph-CH₂—*, —CH₂-Ph-NH—C(S)NR¹—*, —NR¹C(S)—NH-Ph-CH₂—*, —O—, or —NR¹—, wherein “*” indicates the attachment point to L³, and R¹ is hydrogen, optionally substituted C₁-C₆ alkyl, optionally substituted C₁-C₆ heteroalkyl, optionally substituted aryl, or optionally substituted heteroaryl;
L³ is optionally substituted C₁-C₅₀alkyl or optionally substituted C₁-C₅₀ heteroalkyl; and
Z¹ is —CH₂— #, —C(O)— #, —C(S)— #, —OC(O)— #, —C(O)O— #, —NR²C(O)— #, —C(O)NR²— #, or —NR²— #, wherein “#” indicates the attachment point to B, and R² is hydrogen, optionally substituted C₁-C₆ alkyl, optionally substituted C₁-C₆ heteroalkyl, optionally substituted aryl, or optionally substituted heteroaryl,
wherein the antibody or antigen-binding fragment thereof binds to six-transmembrane epithelial antigen of prostate-2 (STEAP2) and competes for binding with an antibody or antigen-binding fragment that comprises
(a) HCDR1 comprising the sequence of SEQ ID NO:1, HCDR2 comprising the sequence of SEQ ID NO:2 and HCDR3 comprising the sequence of SEQ ID NO:3 and LCDR1 comprising the sequence of SEQ ID NO:6, LCDR2 comprising the sequence of SEQ ID NO:7 and LCDR3 comprising the sequence of SEQ ID NO:8;
(b) HCDR1 comprising the sequence of SEQ ID NO:11, HCDR2 comprising the sequence of SEQ ID NO:12 and HCDR3 comprising the sequence of SEQ ID NO:13 and LCDR1 comprising the sequence of SEQ ID NO:16, LCDR2 comprising the sequence of SEQ ID NO:17 and LCDR3 comprising the sequence of SEQ ID NO:18;
(c) HCDR1 comprising the sequence of SEQ ID NO:21, HCDR2 comprising the sequence of SEQ ID NO:22 and HCDR3 comprising the sequence of SEQ ID NO:23 and LCDR1 comprising the sequence of SEQ ID NO:25, LCDR2 comprising the sequence of SEQ ID NO:26 and LCDR3 comprising the sequence of SEQ ID NO:27; or
(d) HCDR1 comprising the sequence of SEQ ID NO:31, HCDR2 comprising the sequence of SEQ ID NO:32 and HCDR3 comprising the sequence of SEQ ID NO:33 and LCDR1 comprising the sequence of SEQ ID NO:35, LCDR2 comprising the sequence of SEQ ID NO:36 and LCDR3 comprising the sequence of SEQ ID NO:37.

Such compounds of Formula I may also be collectively referred to herein as “Compounds”. Embodiments of such compounds may include pharmaceutically acceptable salts thereof unless specified otherwise.

In some embodiments, variable A in Formula I is a chelating moiety selected from the group consisting of DOTA (1,4,7,10-tetraazacyclododecane-1,4,7,10-tetraacetic acid), DOTMA (1R,4R,7R,10R)-α,α,α″,α″′-tetramethyl-1,4,7,10-tetraazacyclododecane-1,4,7,10-tetraacetic acid, DOTAM (1,4,7,10-tetrakis(carbamoylmethyl)-1,4,7,10-tetraazacyclododecane), DOTPA (1,4,7,10-tetraazacyclododecane-1,4,7,10-tetra propionic acid), DO3AM-acetic acid (2-(4,7,10-tris(2-amino-2-oxoethyl)-1,4,7,10-tetraazacyclododecan-1-yl)acetic acid), DOTA-GA anhydride (2,2′,2″-(10-(2,6-dioxotetrahydro-2H-pyran-3-yl)-1,4,7,10-tetraazacyclododecane-1,4,7-triyl)triacetic acid, DOTP (1,4,7,10-tetraazacyclododecane-1,4,7,10-tetra(methylene phosphonic acid)), DOTMP (1,4,6,10-tetraazacyclodecane-1,4,7,10-tetramethylene phosphonic acid, DOTA-4AMP (1,4,7,10-tetraazacyclododecane-1,4,7,10-tetrakis(acetamido-methylenephosphonic acid), CB-TE2A (1,4,8,11-tetraazabicyclo[6.6.2]hexadecane-4,11-diacetic acid), NOTA (1,4,7-triazacyclononane-1,4,7-triacetic acid), NOTP (1,4,7-triazacyclononane-1,4,7-tri(methylene phosphonic acid), TETPA (1,4,8,11-tetraazacyclotetradecane-1,4,8,11-tetrapropionic acid), TETA (1,4,8,11-tetraazacyclotetradecane-1,4,8,11-tetra acetic acid), HEHA (1,4,7,10,13,16-hexaazacyclohexadecane-1,4,7,10,13,16-hexaacetic acid), PEPA (1,4,7,10,13-pentaazacyclopentadecane-N,N′,N″,N″′,N″″-pentaacetic acid), H₄octapa (N,N′-bis(6-carboxy-2-pyridylmethyl)-ethylenediamine-N,N′-diacetic acid), H₂dedpa (1,2-[[6-(carboxy)-pyridin-2-yl]-methylamino]ethane), H₆phospa (N,N′-(methylenephosphonate)-N,N′-[6-(methoxycarbonyl)pyridin-2-yl]-methyl-1,2-diaminoethane), TTHA (triethylenetetramine-N,N,N′,N″,N″′,N′″-hexaacetic acid), DO2P (tetraazacyclododecane dimethanephosphonic acid), HP-D03A (hydroxypropyltetraazacyclododecanetriacetic acid), EDTA (ethylenediaminetetraacetic acid), Deferoxamine, DTPA (diethylenetriaminepentaacetic acid), DTPA-BMA (diethylenetriaminepentaacetic acid-bismethylamide), and porphyrin.

In certain embodiments, variable A in Formula I is DOTA (1,4,7,10-tetraazacyclododecane-1,4,7,10-tetraacetic acid) or a metal complex thereof.

In some embodiments, the compound of Formula I is represented by:

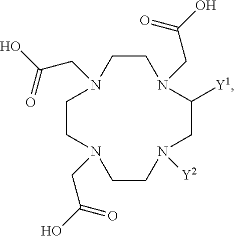

wherein Y¹ is —CH₂OCH₂(L²)ₙ-B, C═O(L²)ₙ-B, or C═S(L²)ₙ-B and Y² is —CH₂CO₂H; or
wherein Y¹ is H and Y² is L¹-(L²)ₙ-B. In certain embodiments, Y¹ is H.

In some embodiments, L¹ is

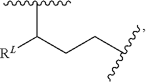

and R^{L} is hydrogen or —CO₂H.

In certain embodiments, X¹ is —C(O)NR¹—*or —NR¹C(O)—*, “*” indicating the attachment point to L³, and R¹ is H.

In certain embodiments, Z¹ is —CH₂—.

In some embodiments, L³ comprises (CH₂CH₂O)₂₋₂₀. In some embodiments, L³ is (CH₂CH₂O)ₘ(CH₂)_{w}, wherein m and w are each independently an integer between 0 and 10 (inclusive), and at least one of m and w is not 0.

In some embodiments, the metal complex comprises a metal selected from the group consisting of Bi, Pb, Y, Mn, Cr, Fe, Co, Zn, Ni, Tc, In, Ga, Cu, Re, a lanthanide, and an actinide.

In some embodiments, the metal complex comprises a radionuclide selected from the group consisting of ⁴⁴Sc, ⁴⁷Sc, ⁵⁵Co, ⁶⁰Cu, ⁶¹Cu, ⁶²Cu, ⁶⁴Cu, ⁶⁷Cu, ⁶⁶Ga ⁶⁷Ga, ⁶⁸Ga, ⁸²Rb, ⁸⁶Y, ⁸⁷Y, ⁸⁹Zr, ⁹⁰Y, ⁹⁷Ru, ⁹⁹Tc, ^{99m}Tc, ¹⁰⁵Rh, ¹⁰⁹Pd, ¹¹¹In, ^{117m}Sn, ¹⁴⁹Pm, ¹⁴⁹Tb, ¹⁵³Sm, ¹⁶⁶Ho, ¹⁷⁷Lu, ¹⁸⁶Re, ¹⁸⁸Re, ¹⁹⁸Au, ¹⁹⁹Au, ²⁰¹Tl, ²⁰³Pb, ²¹¹At, ²¹²Pb, ²¹²Bi, ²¹³Bi, ²²³Ra, ²²⁵Ac, ²²⁷Th, and ²²⁹Th.

In some embodiments, variable A is a metal complex of a chelating moiety. In some such embodiments, the metal complex comprises a radionuclide. In some embodiments, the radionuclide is an alpha emitter, e.g., an alpha emitter selected from the group consisting of Astatine-211 (²¹¹At), Bismuth-212 (²¹²Bi), Bismuth-213 (²¹³Bi), Actinium-225 (²²⁵Ac), Radium-223 (²²³Ra), Lead-212 (²¹²Pb), Thorium-227 (²²⁷Th), and Terbium-149 (¹⁴⁹Tb), or a progeny thereof. In some embodiments, the radionuclide is ⁶⁸Ga, ¹¹¹In, ⁷⁷Lu, or ²²⁵Ac. In some embodiments, the radionuclide is ²²⁵Ac or a progeny thereof.

In some embodiments, compounds of Formula I comprise:

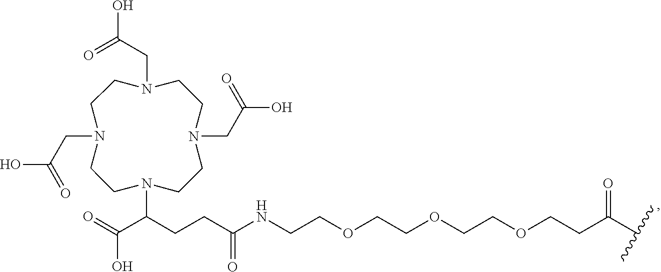 [SMILES: *C(=O)CCOCCOCCOCCNC(=O)CCC(C(=O)O)N1CCN(CC(=O)O)CCN(CC(=O)O)CCN(CC(=O)O)CC1]

or a metal complex thereof, or comprise

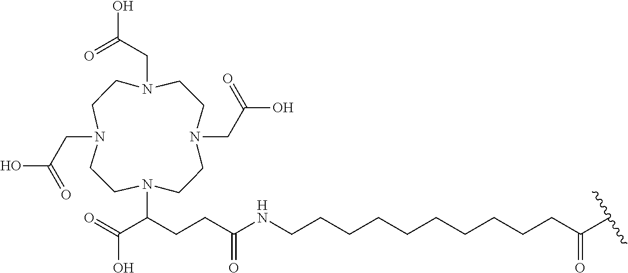 [SMILES: *C(=O)CCCCCCCCCCNC(=O)CCC(C(=O)O)N1CCN(CC(=O)O)CCN(CC(=O)O)CCN(CC(=O)O)CC1]

or a metal complex thereof.

In some embodiments, the compound or a pharmaceutically acceptable salt thereof comprises:

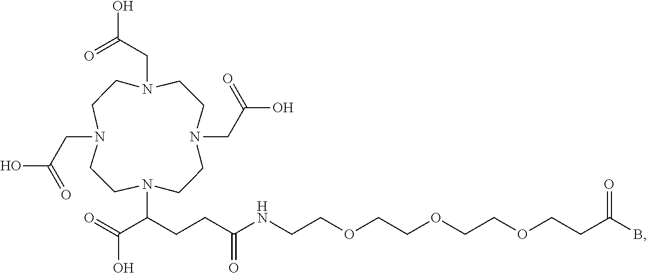

or a metal complex thereof.

In some embodiments, variable A of Formula I is a metal complex of a chelating moiety, and the metal complex comprises a radionuclide. In certain embodiments, the radionuclide is ⁶⁸Ga, ¹¹¹In ¹⁷⁷Lu, or ²²⁵Ac. In certain embodiments, the radionuclide is ²²⁵Ac. In certain embodiments, the radionuclide is an alpha emitter selected from the group consisting of Astatine-211 (²¹¹At), Bismuth-212 (²¹²Bi), Bismuth-213 (²¹³Bi), Actinium-225 (²²⁵Ac), Radium-223 (²²³Ra), Lead-212 (212Pb), Thorium-227 (²²⁷Th), and Terbium-149 (¹⁴⁹Tb), or a progeny thereof. In certain embodiments, the alpha emitter is ²²⁵Ac or a progeny thereof.

In some embodiments, the antibody or antigen-binding fragment thereof comprised within the Compound comprises: HCDR1 comprising, having or consisting of the sequence of SEQ ID NO:1, HCDR2 comprising, having or consisting of the sequence of SEQ ID NO:2 and HCDR3 comprising, having or consisting of the sequence of SEQ ID NO:3 and LCDR1 comprising, having or consisting of the sequence of SEQ ID NO:6, LCDR2 comprising, having or consisting of the sequence of SEQ ID NO:7 and LCDR3 comprising, having or consisting of the sequence of SEQ ID NO:8.

In some embodiments, the antibody or antigen-binding fragment thereof comprised within the Compound comprises: HCDR1 comprising the sequence of SEQ ID NO:1, HCDR2 comprising the sequence of SEQ ID NO:2 and HCDR3 comprising the sequence of SEQ ID NO:3 and LCDR1 comprising the sequence of SEQ ID NO:6, LCDR2 comprising the sequence of SEQ ID NO:7 and LCDR3 comprising the sequence of SEQ ID NO:8.

In some embodiments, the antibody or antigen-binding fragment thereof comprised within the Compound comprises a VH domain having at least 80%, 85%, 90% or 95% sequence identity to the amino acid sequence of SEQ ID NO:4 and a VL domain having at least 80%, 85%, 90% or 95% sequence identity to the amino acid sequence of SEQ ID NO:9. In some embodiments, the antibody or antigen-binding fragment thereof comprises a VH domain having at least 95% sequence identity to the amino acid sequence of SEQ ID NO:4 and a VL domain having at least 95% sequence identity to the amino acid sequence of SEQ ID NO:9. In some embodiments, the antibody or antigen-binding fragment thereof comprises a VH domain comprising the amino acid sequence of SEQ ID NO:4 and a VL domain comprising the amino acid sequence of SEQ ID NO:9.

In some embodiments, the antibody or antigen-binding fragment thereof comprised within the Compound comprises a heavy chain having at least 80%, 85%, 90% or 95% sequence identity to the amino acid sequence of SEQ ID NO:5 and a light chain having at least 80%, 85%, 90% or 95% sequence identity to the amino acid sequence of SEQ ID NO:10. In some embodiments, the antibody or antigen-binding fragment thereof comprises a heavy chain having at least 95% sequence identity to the amino acid sequence of SEQ ID NO:5 and a light chain having at least 95% sequence identity to the amino acid sequence of SEQ ID NO:10. In some embodiments, the antibody or antigen-binding fragment thereof comprises a heavy chain comprising the amino acid sequence of SEQ ID NO:5 and a light chain comprising the amino acid sequence of SEQ ID NO:10.

In some embodiments, the antibody or antigen-binding fragment thereof comprised within the Compound binds to STEAP2 (preferably a human STEAP2) with a binding affinity of between about 0.1 nM to about 40 nM, between about 0.5 nM to about 30 nM, between about 1 nM to about 20 nM, or between about 1 nM to about 10 nM.

In some embodiments, the compound of Formula I, or the pharmaceutically acceptable salt thereof, comprises:

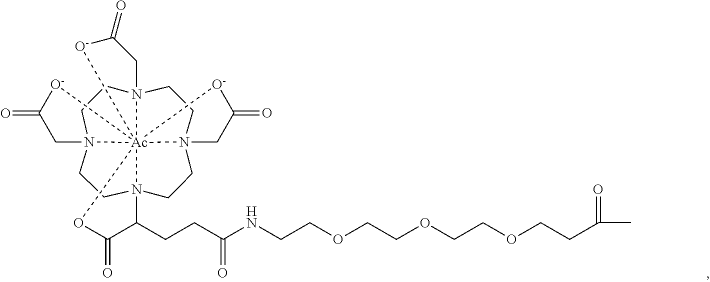

wherein [symbol] is an antibody or an antigen-binding fragment thereof that binds (e.g., specifically binds) to STEAP2. In some embodiments, the antibody or an antigen-binding fragment thereof is linked to A-L¹-(L²)ₙ- via the side-chain amino group of a lysine residue.

In another aspect, the present disclosure also relates to a pharmaceutical composition comprising one of the compounds or pharmaceutically acceptable salt thereof described above and a pharmaceutically acceptable carrier, diluent, or excipient.

Still within the scope of this disclosure is a method of treating a cancer in a subject, said method comprising administering to the subject (e.g., a human) a therapeutically effective amount a compound or pharmaceutically acceptable salt thereof described above or a respective pharmaceutical composition thereof.

In some embodiments, the cancer is a solid tumor cancer selected from the group consisting of prostate cancer (including primary, metastatic, and metastatic castration-resistant forms), bladder cancer (including primary and metastatic forms), breast cancer, colorectal carcinoma, gastric cancer, and other multiple solid tumors where STEAP2 may be overexpressed. Prostate cancers include adenocarcinoma of the prostate, transitional cell carcinoma of the prostate, squamous cell carcinoma of the prostate, small cell prostate cancer, and neuroendocrine differentiated tumors of the prostate.

In some embodiments, the cancer is one of prostate cancer (e.g., metastatic castration-resistant prostate cancer or mCRPC).

In some embodiments, the method of treatment of this disclosure further comprises administering to the subject (e.g., a human) in need thereof an antiproliferative agent, radiation sensitizer, an immunoregulatory or immunomodulatory agent.

Still within the scope of this disclosure is a compound or pharmaceutically acceptable salt thereof or the pharmaceutical composition described above for use in a method of treatment of cancer.

The present disclosure further covers use of a compound or pharmaceutically acceptable salt thereof or the pharmaceutical composition described above in the manufacture of a medicament for the treatment of cancer. In some embodiments, the cancer is a solid tumor cancer selected from the group consisting of prostate cancer, bladder cancer, breast cancer, colorectal carcinoma, and gastric cancer. In some embodiments, the cancer is prostate cancer. In some embodiments, the treatment further comprises an antiproliferative agent, a radiation sensitizer, or an immunomodulatory agent.

### BRIEF DESCRIPTION OF THE DRAWINGS

FIG. 1A shows STEAP2 RNA expression in normal tissues as compared to expression of other tumor associated antigens (TAAs) for prostate cancer, PSMA and STEAP1. Data queried from human protein atlas.

FIG. 1B shows elevated STEAP2 expression across all stages of clinically localized prostate cancer (CaP), ranging from primary diagnosis, primary castration resistant prostate cancer (CRPC), and to lymph node and bone metastasis. Images on the right illustrate two exemplary immunohistochemistry stains (IHC) between CRPC and bone metastasis.

FIG. 2A is a schematic depicting the general structure of bifunctional chelate comprising a chelate, a linker, and a cross-linking group.

FIG. 2B is a schematic depicting the general structure of a bifunctional conjugate comprising a chelate, a linker, and a targeting moiety.

FIG. 2C and FIG. 2D are schematics depicting the structures of [¹⁷⁷Lu]-STEAP2 and [²²⁵Ac]-STEAP2, two exemplary STEAP2 radioimmunoconjugates disclosed herein.

FIG. 3 is a schematic depicting the synthesis of the bifunctional chelate, 4-{[11-oxo-11-(2,3,5,6-tetrafluorophenoxy)undecyl]carbamoyl}-2-[4,7,10-tris(carboxymethyl)-1,4,7,10-tetraazacyclododecan-1-yl]butanoic acid (Compound B). Synthesis of Compound B is described in EXAMPLE 4.

FIG. 4 is a schematic depicting the synthesis of the bifunctional chelate, 4-{[2-(2-{2-[3-oxo-3-(2,3,5,6-tetrafluorophenoxy)propoxy]ethoxy}ethoxy)ethyl]carbamoyl}-2-[4,7,10-tris(carboxymethyl)-1,4,7,10-tetraazacyclododecan-1-yl]butanoic acid (Compound C). Synthesis of Compound C is described in EXAMPLE 5.

FIG. 5 is a schematic depicting the conjugation and radiolabeling for synthesis of [¹⁷⁷Lu]-STEAP2 conjugate (e.g., [¹⁷⁷Lu]-STEAP2 (40A3-LO11) and [¹⁷⁷Lu]-STEAP2 (40A3-L⁰¹⁴) described below). See EXAMPLE 6.

FIGS. 6A-6C show in vitro binding of [¹⁷⁷Lu]-STEAP2 to three different cell lines: LNCaP cells (FIG. 6A), C4-2 cells (FIG. 6B), and 22RV1 cells (FIG. 6C) with varying expression levels of STEAP2 in the absence (total binding) or presence (nonspecific binding) of 4 μM unlabeled antibody. Specific binding was calculated by subtraction of nonspecific binding from total binding. Values shown are the mean±SEM (n=3). See EXAMPLE 7.

FIGS. 7A-7C show the results of internalization (or in vitro residualization) of [¹⁷⁷Lu]-STEAP2 (10 nM) in three different cell lines: LNCaP cells (FIG. 7A), C4-2 cells (FIG. 7B), and 22RV1 cells (FIG. 7C) with varying expression levels of STEAP2 at 2 h and 24 h post incubation. Values shown are the mean±SEM (n=3). See EXAMPLE 8.

FIGS. 8A-8B show the results of biodistribution studies in a various animal models and injected with [¹⁷⁷Lu]-STEAP2 in male athymic NCr nude mice (n=3) with subcutaneous xenografts at specified timepoint following intravenous injection. Percentage injected dose per gram of tissue (% ID/g) is plotted on the x-axis and is shown for blood, bone, brain, heart, intestines, kidneys, lungs, liver, pancreas, spleen, stomach, skin, urine and bladder, and tumor at 4, 24, 72, 96, 168, and 336 hours. See EXAMPLE 9.

FIGS. 9A-9B show the results of an in vivo efficacy study, including relative body-weight, in male athymic NCr nude mice (n=5) following intravenous injection of [²²⁵Ac]-STEAP2 (50-400 nCi/2 μg), cold antibody and vehicle. See EXAMPLE 10.

FIG. 9C shows the results of an in vivo efficacy study, including relative body-weight, in male NOD-SCID mice (n=4) following intravenous injection of [²²⁵Ac]-STEAP2 (50-100 nCi/2 g), isotype antibody and vehicle. See EXAMPLE 10.

FIG. 9D shows the results of the CTG-3167 PDX efficacy study, including body weight changes, in male NOG mice (n=3) following intravenous injection of [²²⁵Ac]-STEAP2 (50-100 nCi) or isotype antibody as compared to the untreated cohort. See EXAMPLE 10.

### DETAILED DESCRIPTION

Radioimmunoconjugates are designed to target a protein or receptor that is upregulated in a disease state to deliver a radioactive payload to damage and kill cells of interest (radioimmunotherapy). Delivering a radioactive payload results in targeted alpha, beta, gamma particle or Auger electron emission that can cause direct effects to DNA (such as single or double stranded DNA breaks) or indirect effects such as by-stander or crossfire effects.

Radioimmunoconjugates typically contain a biological targeting moiety (e.g., an antibody or antigen-binding fragment thereof that is capable of specifically binding to STEAP2), a radionuclide (e.g., an alpha or beta emitter), and a molecule that links the two. Conjugates are formed when a bifunctional chelate is appended to the biological targeting moiety so that structural alterations are minimal while maintaining target affinity. Once radiolabelled, the final radioimmunoconjugate is formed.

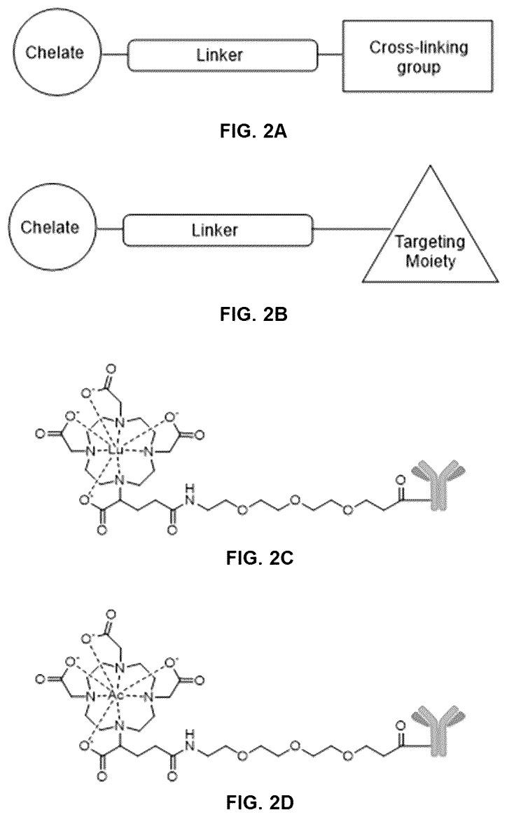

Bifunctional chelates structurally contain a chelate, a linker, and a cross-linking group (FIG. 2A). When developing new bifunctional chelates, most efforts focus on the chelating portion of the molecule. Several examples of bifunctional chelates have been described with various cyclic and acyclic structures conjugated to a targeted moiety. [Bioconjugate Chem. 2000, 11, 510-519; Bioconjugate Chem. 2012, 23, 1029-1039; Mol Imaging Biol. 2011, 13, 215-221, Bioconjugate Chem. 2002, 13, 110-115.]

One of the key factors of developing safe and effective radioimmunoconjugates is maximizing efficacy while minimizing off-target toxicity in normal tissue. While this statement is one of the core tenets of developing new drugs, the application to radioimmunotherapeutics presents new challenges. Radioimmunoconjugates do not need to block a receptor, as needed with a therapeutic antibody, or release the cytotoxic payload intracellularly, as required by an antibody drug conjugate (“ADC”), to have therapeutic efficacy. However, the emission of the toxic particle is an event that occurs as a result of first-order (radioactive) decay and can occur at random anywhere inside the body after administration. Once the emission occurs, damage could occur to surrounding cells within the range of the emission leading to the potential of off-target toxicity. Therefore, limiting exposure of these emissions to normal tissue is the key to developing new therapeutic radioimmunoconjugates.

One potential method for reducing off-target exposure is to remove the radioactivity more effectively from the body (e.g., from normal tissue in the body). One mechanism is to increase the rate of clearance of the biological targeting agent. Without being bound by theory, this approach may require identifying ways to shorten the half-life of the biological targeting agent, which is not well described for biological targeting agents. Regardless of the mechanism, increasing drug clearance will also negatively impact the pharmacodynamics/efficacy in that the more rapid removal of drug from the body will lower the effective concentration at the site of action, which, in turn, would require a higher total dose and would not achieve the desired results of reducing total radioactive dose to normal tissue.

Other efforts have focused on accelerating the metabolism of the portion of the molecule that contains the radioactive moiety. To this end, some efforts have been made to increase the rate of cleavage of the radioactivity from the biological targeting agents using what have been termed “cleavable linkers”. Cleavable linkers, however, have been taken on different meaning as it relates to radioimmunoconjugates. Cornelissen, et al. has described cleavable linkers as those by which the bifunctional chelate attaches to the biologic targeting agent through a reduced cysteine, whereas others have described the use of enzyme-cleavable systems that require the co-administration of the radioimmunoconjugate with a cleaving agent/enzyme to release [Mol Cancer Ther. 2013, 12(11), 2472-2482; Methods Mol Biol. 2009, 539, 191-211; Bioconjug Chem. 2003, 14(5), 927-33]. These methods either change the nature of the biological targeting moiety, in the case of the cysteine linkage, or are not practical from a drug development perspective (enzyme cleavable systems) since, in the case of the citations provided, require the administration of two agents.

The present disclosure provides, among other things, compounds, e.g., radioimmunoconjugates, that are more effectively eliminated from the body after catabolism and/or metabolism, thereby more effectively eliminating radioactivity from the body while maintaining therapeutic efficacy. This unexpected superiority is achieved, at least in part, by making modifications to the linker region of the bifunctional chelate.

Disclosed immunoconjugates may, in some embodiments, achieve a reduction of total body radioactivity, for example, by increasing the extent of excretion of the catabolic/metabolic products while maintaining the pharmacokinetics of the intact molecule when compared to known bifunctional chelates. In some embodiments, this reduction in radioactivity results from the clearance of catabolic/metabolic by-products without impacting other in vitro and in vivo properties such as binding specificity, cellular retention, and tumor uptake in vivo. Thus, in some embodiments, provided compounds achieve reduced radioactivity in the human body while maintaining on-target activity.

In some embodiments, an anti-STEAP2 antibody radiopharmaceutical targeted alpha therapy (TAT) may be used as a stand-alone treatment or in combination with other therapies such as check point inhibitors, chemotherapy, DNA damage repair inhibitors or other therapeutic modalities to target clinical indications, e.g., those expressing STEAP2. The treatment will activate a variety of pathways to drive anti-tumor processes by causing alpha radiation targeted tumor cell death via DNA damage, leading to the stimulation of the host immune system towards tumor cells, and ultimately resulting in the killing/eradication of tumor cells.

The evaluation of Lu-177-conjugated anti-STEAP2 antibody biodistribution in mice with STEAP2-positive tumors revealed the specificity of radioconjugated anti-STEAP2 antibody and its uptake in STEAP2-positive tumors. Furthermore, single dose therapy with anti-STEAP2 antibody conjugated with Ac-225 led to tumor regression and overall survival benefit, suggesting specificity and effectiveness of anti-STEAP2 targeted alpha therapy.

Disclosed herein are certain radiopharmaceuticals and their use for treating cancers (e.g., prostate cancer). Specifically, in some embodiments, the present disclosure provides anti-STEAP2 antibody-based radiopharmaceuticals (antibody-conjugated targeted alpha therapy) using actinium-225, lutetium-177, Indium-111 or other suitable therapeutic radioisotope payload targeted to STEAP2 expressing cancer indications.

### Definitions

As used herein, the term “bind” or “binding” of a targeting moiety means an at least temporary interaction or association with or to a target molecule, e.g., human STEAP2, as described herein.

The terms “bifunctional chelate,” as used herein, refers to a compound that comprises a chelate, a linker, and a cross-linking group. See, e.g., FIG. 2A. A “cross-linking group” is a reactive group that is capable of joining two or more molecules, e.g., joining a bifunctional chelate and a targeting moiety, by a covalent bond.

The term “bifunctional conjugate,” as used herein, refers to a compound that comprises a chelate or metal complex thereof, a linker, and a targeting moiety, e.g., an antibody or antigen-binding fragment thereof. See, e.g., FIG. 2B.

The term “cancer,” as used herein, refers to any disease caused by the proliferation of malignant neoplastic cells, such as tumors, neoplasms, carcinomas, sarcomas, leukemias, and lymphomas. In some embodiments, a cancer of the present disclosure comprises cells (e.g., tumor cells) expressing STEAP2, such as, but not limited to, lung cancer, colorectal cancer, pancreatic cancer, or head and neck cancer.

The term “chelate,” as used herein, refers to an organic compound or portion thereof that can be complexed with a central metal or radiometal atom at two or more points.

The term “conjugate,” as used herein, refers to a molecule that contains a chelating group or metal complex thereof, a linker group, and which optionally contains a targeting moiety, e.g., an antibody or antigen-binding fragment thereof.

“Affinity” refers to the strength of noncovalent interactions between a single binding site of a molecule (e.g., of an antibody or antigen-binding fragment thereof) and a single binding site (e.g., epitope) on the molecule's binding partner (e.g., an antigen). The affinity of a molecule X for its partner Y can generally be represented by the dissociation constant (Kd). Affinity can be measured by common methods known in the art, including those described herein. Specific illustrative and exemplary embodiments for measuring binding affinity are described in the following.

“Affinity matured” in reference to an antibody means an antibody with one or more alterations in one or more complementarity determining regions (CDRs), compared to a parent antibody which does not possess such alterations, such alterations resulting in an improvement in the affinity of the antibody for the antigen.

“Anti-STEAP2 antibody” refers to an antibody that is capable of binding to STEAP2. In some embodiments, the anti-STEAP2 antibody binds specifically to STEAP2. That means that the extent of binding of the anti-STEAP2 antibody to an unrelated, non-protein-X protein is less than about 10% of the binding of the antibody to STEAP2.

“Antibody” is used in the broadest sense and encompasses various antibody structures, including but not limited to monoclonal antibodies, polyclonal antibodies, multispecific antibodies (e.g., bispecific antibodies). The structures and locations of immunoglobulin variable domains, e.g., CDRs, may be defined using well known numbering schemes, e.g., the Kabat numbering scheme, the Chothia numbering scheme, or a combination of Kabat and Chothia (see, e.g., Sequences of Proteins of Immunological Interest, U.S. Department of Health and Human Services (1991), eds. Kabat et al.; Al Lazikani et al., (1997) J. Mol. Bio. 273:927 948); Kabat et al., (1991) Sequences of Proteins of Immunological Interest, 5^{th} edit., NIH Publication no. 91-3242 U.S. Department of Health and Human Services; Chothia et al., (1987) J. Mol. Biol. 196:90 1-917; Chothia et al., (1989) Nature 342:877-883; and Al-Lazikani et al., (1997) J. Mal. Biol. 273:927-948. In preferred embodiments, variable domains, e.g., CDRs, are defined according to the Kabat numbering scheme.

“Antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. An exemplary competition assay includes a homogeneous time-resolved fluorescence (HTRF) assay.

“Antigen-binding fragment” refers to a molecule other than an intact antibody that comprises a portion of the intact antibody that binds to the antigen to which the intact antibody binds. Examples of antigen-binding fragments include, but are not limited to, Fv, Fab, Fab′, F(ab′)2, Fab′-SH, diabodies, triabodies, tetrabodies, linear antibodies, single-chain antibody molecules (e.g., scFv), and multispecific antibodies formed from antigen-binding fragments. For a review of certain antigen-binding fragments, see Hudson et al. Nat. Med. 9:129-134 (2003). For a review of scFv fragments, see, e.g., Pluckthün, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)2 fragments comprising salvage receptor binding epitope residues and having increased in vivo half-life, see U.S. Pat. No. 5,869,046. Diabodies are antigen-binding fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003). Single-domain antibodies are antigen-binding fragments comprising all or a portion of the heavy chain variable domain or all or a portion of the light chain variable domain of an antibody. Antigen-binding fragments can be made by various techniques, including but not limited to proteolytic digestion of an intact antibody as well as production by recombinant host cells (e.g. E. coli or phage).

“Compete” or “competes” or “competes for binding to” used herein in regard to an antibody or antigen-binding fragment thereof, means that a first antigen-binding domain binds to an epitope of a protein (e.g., STEAP2) in a manner sufficiently similar to the binding of a second antibody or antigen-binding fragment thereof, such that the result of binding of the first antibody or antigen-binding fragment thereof with its epitope is detectably decreased in the presence of the second antibody or antigen-binding fragment thereof compared to the binding of the first antibody or antigen-binding fragment thereof in the absence of the second antibody or antigen-binding fragment thereof. “Cross-competes” means that, as well as the second antibody or antigen-binding fragment competing for binding to an antigen with a first antibody which has been pre-incubated with the antigen, the first antibody also competes for binding to the antigen when the second antibody is pre-incubated with said antigen.

“Complementarity determining regions” and “CDRs” are used herein to refer to the amino acid residues of an antibody or antigen-binding fragment that provide the primary contact residues for antigen binding.

“Class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called a, 8, E, y, and 11, respectively.

“Epitope” includes any protein determinant capable of specific binding to an antibody or antigen-binding fragment thereof. Epitope determinants generally consist of chemically active surface groupings of molecules such as amino acids or carbohydrate or sugar side chains and can have specific three-dimensional structural characteristics, as well as specific charge characteristics. An epitope may be “linear” or “conformational.” Conformational and linear epitopes are distinguished in that the binding to the former but not the latter is generally lost in the presence of denaturing solvents.

“Fc region” herein is used to define a C-terminal region of an immunoglobulin heavy chain that contains at least a portion of the constant region. The term includes native sequence Fc regions and variant Fc regions. In some embodiments, a variant Fc region may be modified compared to a wild-type constant region. That is, the Fc region may comprise alterations or modifications to one or more of the three heavy chain constant domains (CH1, CH2 or CH3). Example modifications include additions, deletions or substitutions of one or more amino acids in one or more domains. Such modifications may also be present in the constant light (CL) domain. Such changes may be included to optimize effector function, half-life, etc. Unless otherwise specified herein, numbering of amino acid residues in the Fc region or constant region is according to the EU numbering system, also called the EU index, as described in Kabat et al., Sequences of Proteins of Immunological Interest, 5^{th} Ed. Public Health Service, National Institutes of Health, Bethesda, MD, 1991.

“Framework” or “FW” refers to variable domain residues other than complementarity determining region (CDR) residues. The FR of a variable domain generally consists of four FW domains: FW1, FW2, FW3, and FW4. Accordingly, the CDR and FW sequences generally appear in the following sequence in VH: FW1-HCDR1-FW2-HCDR2-FW3-HCDR3-FW4, and the CDR and FW sequences generally appear in the following sequence in VL: FW1-LCDR1-FW2-LCDR2-FW3-LCDR3-FW4.

“Full-length”, “intact” and “whole” when used together with the term “antibody” are used interchangeably. They refer to an antibody having a structure substantially similar to a native antibody structure or having heavy chains that contain an Fc region as defined herein.

“Host cell” refers to cells into which exogenous nucleic acid has been introduced. Host cell includes the progeny of such cells and transformed cells, which include the primary transformed cells and progeny derived therefrom without regard to the passage number.

“Human antibody,” as used herein, includes antibodies having variable regions in which both the framework and CDR regions are derived from sequences of human origin.

Furthermore, if the antibody contains a constant region, the constant region is also derived from such human sequences, e.g., human germline sequences, or mutated versions of human germline sequences or antibody containing consensus framework sequences derived from human framework sequences analysis, for example, as described in Knappik, et al. (2000. J Mol Biol296, 57-86). Human antibodies of the disclosure may include amino acid residues not encoded by human sequences (e.g., mutations introduced by random or site-specific mutagenesis in vitro or by somatic mutation in vivo, or a conservative substitution to promote stability or manufacturing). However, the term “human antibody” as used herein, is not intended to include antibodies in which CDR sequences derived from the germline of another mammalian species, such as a mouse, have been grafted onto human framework sequences.

A “chimeric” antibody refers to an antibody comprising a portion of the heavy and/or light chain which is identical or homologous to corresponding sequences in antibodies derived from a particular species or belonging to a particular antibody class or subclass (e.g., chimeric humanized, class-switched antibodies), while the remainder of the chain(s) is identical or homologous to corresponding sequences in antibodies derived from another species or belonging to another antibody class or subclass (Morrison et al., Proc. Natl. Acad. Sci. USA, 81:6851-6855 (1984)).

A “humanized” antibody refers to an antibody comprising amino acid residues from non-human CDRs and amino acid residues from human FWs. A humanized antibody may comprise substantially all of at least one, and typically two, variable domains, in which all or substantially all of the CDRs correspond to those of a non-human antibody, and all or substantially all of the FWs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

“Isolated” antibody is an antibody which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

“Isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extra-chromosomally or at a chromosomal location that is different from its natural chromosomal location.

The term “subject” refers to an animal, human or non-human, to whom treatment according to the methods of the present disclosure is provided. Veterinary and nonveterinary applications are contemplated. The term includes, but is not limited to, mammals, e.g., humans, other primates, pigs, rodents such as mice and rats, rabbits, guinea pigs, hamsters, cows, horses, cats, dogs, sheep and goats. Typical subjects include humans, farm animals, and domestic pets such as cats and dogs. The preferred subject is a human.

As used herein, the term “compound,” is meant to include all stereoisomers, geometric isomers, tautomers, and/or salts thereof of the structures disclosed herein.

The compounds recited or described herein can be asymmetric (e.g., having one or more stereocenters). All stereoisomers, such as enantiomers and diastereomers, are intended unless otherwise indicated. Compounds discussed in the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis.

As used herein, “detection agent” refers to a molecule or atom which is useful in diagnosing a disease by locating the cells containing the antigen. Various methods of labeling polypeptides with detection agents are known in the art. Examples of detection agents include, but are not limited to, radioisotopes and radionuclides, dyes (such as with the biotin-streptavidin complex), contrast agents, luminescent agents (e.g., fluorescein isothiocyanate or FITC, rhodamine, lanthanide phosphors, cyanine, and near IR dyes), and magnetic agents, such as gadolinium chelates.

As used herein, the term “radionuclide,” refers to an atom capable of undergoing radioactive decay (e.g., ³H, ¹⁴C, ¹⁵N, ¹⁸F, ³⁵S, ⁴⁴Sc, ⁴⁷Sc, ⁵⁵Co, ⁶⁰Cu, ⁶¹Cu, ⁶²Cu, ⁶⁴Cu, ⁶⁷Cu, ⁷⁵Br, ⁷⁶Br, ⁷⁷Br, ⁸⁹Zr, ⁸⁶Y, ⁸⁷Y, ⁹⁰Y, ⁹⁷Ru, ⁹⁹Tc, ^{99m}Tc, ¹⁰⁵Rh, ¹⁰⁹Pd, ¹¹¹In, ¹²³I, ¹²⁴I, ¹²⁵I, ¹³¹I, ¹⁴⁹Pm, ¹⁴⁹Tb ¹⁵³Sm, ¹⁶⁶Ho, ¹⁷⁷Lu, ¹⁸⁶Re, ¹⁸⁸Re, ¹⁹⁸Au, ¹⁹⁹Au, ²⁰³Pb, ²¹¹At, ²¹²Pb, ²¹²Bi, ²¹³Bi, ²²³Ra, ²²⁵Ac, ²²⁷Th, ²²⁹Th ⁶⁶Ga, ⁶⁷Ga, ⁶⁸Ga, ⁸²Rb, ^{117m}Sn, ²⁰¹Tl). The terms radioactive nuclide, radioisotope, or radioactive isotope may also be used to describe a radionuclide. Radionuclides may be used as detection agents, as described herein. In some embodiments, the radionuclide may be used as therapeutic agents, e.g., an alpha-emitting radionuclide.

The term an “effective amount” of an agent (e.g., any of the foregoing conjugates), as used herein, is that amount sufficient to effect beneficial or desired results, such as clinical results, and, as such, an “effective amount” depends upon the context in which it is being applied. For example, in therapeutic applications, an “effective amount” may be an amount sufficient to cure or at least partially arrest the symptoms of the disorder and its complications, to substantially improve at least one symptom associated with the disease or a medical condition, to slow the progression of symptoms of the disorder and its complications, and/or to slow the progression of at least one symptom associated with the disease or a medical condition.

Typically, an “effective amount” in the context of the present disclosure is an amount of a radioimmunoconjugate disclosed herein, e.g., an Ac-225-radioimmunoconjugate, that produces at least some measurable therapeutic response or desired effect in some fraction of the patient to whom it is administered. For example, in the treatment of cancer, an agent or compound that decreases, prevents, delays, suppresses, or arrests any symptom of the disease or condition would be therapeutically effective. A therapeutically effective amount of an agent or compound is not required to cure a disease or condition but may, for example, provide a treatment for a disease or condition such that the onset of the disease or condition is delayed, hindered, or prevented, such that the disease or condition symptoms are ameliorated, or such that the term of the disease or condition is changed. For example, the disease or condition may become less severe and/or recovery is accelerated in an individual. An effective amount may be administered by administering a single dose or multiple (e.g., at least two, at least three, at least four, at least five, or at least six) doses.

The term “immunoconjugate,” as used herein, refers to a conjugate that includes a targeting moiety, such as an antibody (or antigen-binding fragment thereof), nanobody, affibody, or a consensus sequence from Fibronectin type III domain. In some embodiments, the immunoconjugate comprises an average of at least 0.10 conjugates per targeting moiety (e.g., an average of at least 0.2, 0.3, 0.4, 0.5, 0.6, 0.7, 0.8, 0.9, 1, 2, 3, 4, 5, 6, 7, or 8 conjugates per targeting moiety). In some embodiments, the immunoconjugate comprises an average of at least or about 0.20 conjugates per targeting moiety. In some embodiments, the immunoconjugate comprises an average of at least or about 0.30 conjugates per targeting moiety. In some embodiments, the immunoconjugate comprises an average of at least or about 0.40 conjugates per targeting moiety. In some embodiments, the immunoconjugate comprises an average of at least or about 0.50 conjugates per targeting moiety. In some embodiments, the immunoconjugate comprises an average of at least or about 0.60 conjugates per targeting moiety. In some embodiments, the immunoconjugate comprises an average of at least or about 0.70 conjugates per targeting moiety. In some embodiments, the immunoconjugate comprises an average of at least or about 0.80 conjugates per targeting moiety. In some embodiments, the immunoconjugate comprises an average of at least or about 0.90 conjugates per targeting moiety. In some embodiments, the immunoconjugate comprises an average of at least or about 1 conjugate per targeting moiety.

The term “radioconjugate,” as used herein, refers to any conjugate that includes a radioisotope or radionuclide, such as any of the radioisotopes or radionuclides described herein.

The term “radioimmunoconjugate,” as used herein, refers to any immunoconjugate that includes a radioisotope or radionuclide, such as any of the radioisotopes or radionuclides described herein. A radioimmunoconjugate provided in the present disclosure typically refers to a bifunctional conjugate that comprises a metal complex formed from a radioisotope or radionuclide.

The term “radioimmunotherapy,” as used herein, refers a method of using a radioimmunoconjugate to produce a therapeutic effect. In some embodiments, radioimmunotherapy may include administration of a radioimmunoconjugate to a subject in need thereof, wherein administration of the radioimmunoconjugate produces a therapeutic effect in the subject. In some embodiments, radioimmunotherapy may include administration of a radioimmunoconjugate to a cell, wherein administration of the radioimmunoconjugate kills the cell. Wherein radioimmunotherapy involves the selective killing of a cell, in some embodiments the cell is a cancer cell in a subject having cancer.

The term “pharmaceutical composition” as used herein, represents a composition containing a radioimmunoconjugate described herein formulated with a pharmaceutically acceptable excipient. In some embodiments, the pharmaceutical composition is manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein.

A “pharmaceutically acceptable excipient,” as used herein, refers any ingredient other than the compounds described herein (for example, a vehicle capable of suspending or dissolving the active compound) and having the properties of being nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, radioprotectants, sorbents, suspending or dispersing agents, sweeteners, or waters of hydration. Exemplary excipients include, but are not limited to: ascorbic acid, histidine, phosphate buffer, butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

The term “pharmaceutically acceptable salt,” as use herein, represents those salts of the compounds described here that are, within the scope of sound medical judgment, suitable for use in contact with the tissues of humans and animals without undue toxicity, irritation, or allergic response. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. Salts can be prepared in situ during the final isolation and purification of the compounds described herein or separately by reacting the free base group with a suitable organic acid.

The compounds of the disclosure may have ionizable groups so as to be capable of preparation as pharmaceutically acceptable salts. These salts may be acid addition salts involving inorganic or organic acids or the salts may, in the case of acidic forms of the compounds of the disclosure be prepared from inorganic or organic bases. Frequently, the compounds are prepared or used as pharmaceutically acceptable salts prepared as addition products of pharmaceutically acceptable acids or bases. Suitable pharmaceutically acceptable acids and bases are well-known in the art, such as hydrochloric, sulphuric, hydrobromic, acetic, lactic, citric, or tartaric acids for forming acid addition salts, and potassium hydroxide, sodium hydroxide, ammonium hydroxide, caffeine, various amines for forming basic salts. Methods for preparation of the appropriate salts are well-established in the art.

Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, among others. Representative alkali or alkaline earth metal salts include sodium, lithium, potassium, calcium, and magnesium, as well as nontoxic ammonium, quaternary ammonium, and amine cations, including, but not limited to ammonium, tetramethylammonium, tetraethylammonium, methylamine, dimethylamine, trimethylamine, triethylamine, and ethylamine.

The term “polypeptide,” as used herein, refers to a string of at least two amino acids attached to one another by a peptide bond. In some embodiments, a polypeptide may include at least 3-5 amino acids, each of which is attached to others by way of at least one peptide bond. Those of ordinary skill in the art will appreciate that polypeptides can include one or more “non-natural” amino acids or other entities that nonetheless are capable of integrating into a polypeptide chain. In some embodiments, a polypeptide may be glycosylated, e.g., a polypeptide may contain one or more covalently linked sugar moieties. In some embodiments, a single “polypeptide” (e.g., an antibody polypeptide) may comprise two or more individual polypeptide chains, which may in some cases be linked to one another, for example by one or more disulfide bonds or other means.

By “substantial identity” or “substantially identical” is meant a polypeptide sequence that has the same polypeptide sequence, respectively, as a reference sequence, or has a specified percentage of amino acid residues, respectively, that are the same at the corresponding location within a reference sequence when the two sequences are optimally aligned. For example, an amino acid sequence that is “substantially identical” to a reference sequence has at least 80%, 85%, 90%, 95%, 96%, 97%, 98%, 99%, or 100% identity to the reference amino acid sequence. For polypeptides, the length of comparison sequences will generally be at least 5, 6, 7, 8, 9, 10, 11, 12, 13, 14, 15, 16, 17, 18, 19, 20, 25, 50, 75, 90, 100, 150, 200, 250, 300, or 350 contiguous amino acids (e.g., a full-length sequence). Sequence identity may be measured using sequence analysis software on the default setting (e.g., Sequence Analysis Software Package of the Genetics Computer Group, University of Wisconsin Biotechnology Center, 1710 University Avenue, Madison, WI 53705). Such software may match similar sequences by assigning degrees of homology to various substitutions, deletions, and other modifications.

As used herein, and as well understood in the art, “to treat” a condition or “treatment” of the condition (e.g., the conditions described herein such as cancer) is an approach for obtaining beneficial or desired results, such as clinical results. Beneficial or desired results can include, but are not limited to, alleviation or amelioration of one or more symptoms or conditions; diminishment of extent of disease, disorder, or condition; stabilized (i.e., not worsening) state of disease, disorder, or condition; preventing spread of disease, disorder, or condition; delay or slowing the progress of the disease, disorder, or condition; amelioration or palliation of the disease, disorder, or condition; and remission (whether partial or total), whether detectable or undetectable. “Palliating” a disease, disorder, or condition means that the extent and/or undesirable clinical manifestations of the disease, disorder, or condition are lessened and/or time course of the progression is slowed or lengthened, as compared to the extent or time course in the absence of treatment.

As used herein, the term “about” or “approximately,” when used in reference to a quantitative value, includes the recited quantitative value itself, unless specifically stated otherwise. As used herein, the term “about” or “approximately” refers to a ±¹⁰% variation from the recited quantitative value unless otherwise indicated or inferred from the context.

As used herein, the term “targeting moiety” refers to any molecule or any part of a molecule that is capable of binding to a given target. The term, “STEAP2 targeting moiety” refers to a targeting moiety (e.g., an antibody or antigen-binding fragment thereof) that is capable of binding to STEAP2, e.g., anti-STEAP2 antibody.

As used herein, a “functional variant” binds to the same target antigen as the reference antibody, and exhibits the same antigen cross-reactivity as the reference antibody. The functional variants may have a different affinity for the target antigen when compared to the reference antibody, but substantially the same affinity is preferred. A functional variant may be referred to as a “variant antibody”.

As used herein, the term “functional fragment,” when used to refer to a STEAP2 fragment, refers to N-terminally and/or C-terminally truncated STEAP2 or protein domains thereof. Unless otherwise specified, a fragment described herein is a functional fragment. Unless otherwise noted, fragments of STEAP2 used in accordance with embodiments described herein retain the capability of the full-length STEAP2 to be recognized and/or bound by an STEAP2-targeting moiety as described in the present disclosure.

The use of the terms “a” and “an” and “the” and similar referents in the context of describing the disclosure (especially in the context of the claims) are to be construed to cover both the singular and the plural, unless otherwise indicated herein or clearly contradicted by context.

Unless specifically stated or obvious from context, as used herein the term “or” is understood to be inclusive and covers both “or” and “and”.

The term “and/or” where used herein is to be taken as specific disclosure of each of the specified features or components with or without the other.

The terms “comprising”, “having”, “including”, and “containing” are to be construed as open-ended terms (i.e., meaning “including, but not limited to”) unless otherwise noted. The term “consisting of” is to be construed as close-ended.

### Compounds, e.g., Immunoconjugates or Radioimmunoconjugates

In one aspect, this disclosure provides compounds, e.g., immunoconjugates or radioimmunoconjugates, comprising the following structure, or pharmaceutically acceptable salts thereof:

A-L¹-(L²)ₙ-B Formula I
wherein
A is a chelating moiety or a metal complex thereof,
B is an antibody or antigen-binding fragment thereof that is capable of binding to STEAP2, wherein the antibody or antigen-binding fragment thereof is featured as disclosed herein;
L¹ is a bond, C═O, C═S, optionally substituted C₁-C₆ alkyl, optionally substituted C₁-C₆ heteroalkyl, optionally substituted aryl, or optionally substituted heteroaryl;
n is an integer between 1 and 5 (inclusive); and
L² each independently has the structure of Formula II:

—X¹-L³-Z¹— Formula II
wherein
   X¹ is —C(O)NR¹—*, —NR¹C(O)—*, —C(S)NR¹—*, —NR¹C(S)—*, —OC(O)NR¹—*, —NR¹C(O)O—*, —NR¹C(O)NR¹—*, —CH₂-Ph-C(O)NR¹—*, —NR¹C(O)-Ph-CH₂—*, —CH₂-Ph-NH—C(S)NR¹—*, —NR¹C(S)—NH-Ph-CH₂—*, —O—*, or —NR¹—*; wherein “*” indicates the attachment point to L³, and R¹ is hydrogen, optionally substituted C₁-C₆ alkyl, optionally substituted C₁-C₆ heteroalkyl, or optionally substituted aryl or heteroaryl;
   L³ is optionally substituted C₁-C₅₀alkyl or optionally substituted C₁-C₅₀ heteroalkyl; and
   Z¹ is —CH₂—#, —C(O)—#, —C(S)—#, —OC(O)—#, —C(O)O—#, —NR²C(O)—#, —C(O)NR²—#, or —NR²—#, wherein “#” indicates the attachment point to B, and R² is hydrogen, optionally substituted C₁-C₆ alkyl, optionally substituted C₁-C₆ heteroalkyl, optionally substituted aryl, or optionally substituted heteroaryl.

Typical substituents of alkyl, heteroalkyl, aryl, or heteroaryl include, but are not limited to halo (e.g., F, Cl, Br, I), OH, CN, nitro, amino, C₁₋₆ alkyl, C₂₋₆ alkenyl, C₂₋₆ alkynyl, C₃₋₈ cycloalkyl, C₁₋₆ heteroalkyl, C₁₋₆ heterocycloalkyl, haloalkyl (e.g., CF₃), alkoxy (e.g., OCH₃), alkylamino (e.g., NH₂CH₃), sulfonyl, aryl, and heteroaryl.

In some embodiments, the compound or pharmaceutically acceptable salt thereof (e.g., immunoconjugate or radioimmunoconjugate) has or comprises:

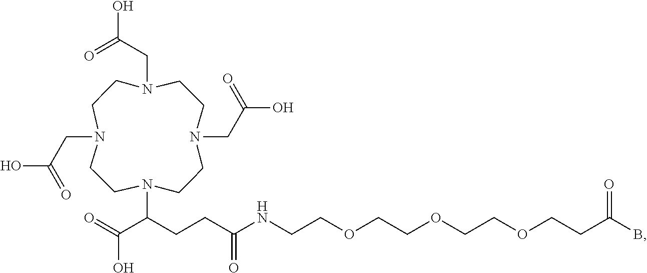

wherein B is a STEAP2 antibody or antigen-binding fragment thereof as disclosed herein.

In some embodiments, the compound or pharmaceutically acceptable salt thereof of Formula I comprises:

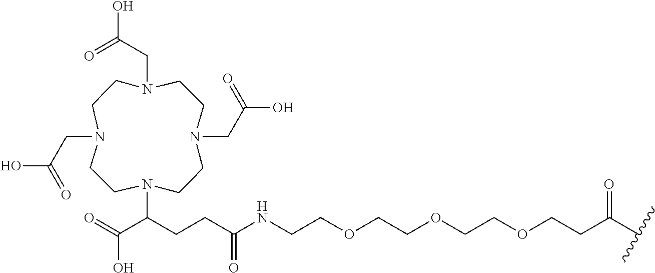 [SMILES: *C(=O)CCOCCOCCOCCNC(=O)CCC(C(=O)O)N1CCN(CC(=O)O)CCN(CC(=O)O)CCN(CC(=O)O)CC1]

or a metal complex thereof or the compound comprises

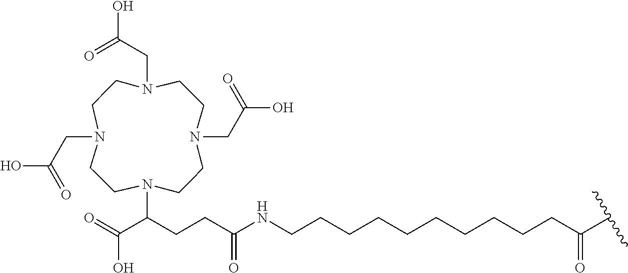 [SMILES: *C(=O)CCCCCCCCCCNC(=O)CCC(C(=O)O)N1CCN(CC(=O)O)CCN(CC(=O)O)CCN(CC(=O)O)CC1]

or a metal complex thereof.

In some embodiments, provided compounds or pharmaceutically acceptable salt thereof (e.g., immunoconjugates or radioimmunoconjugates) are capable of binding to different cell lines with varying expression levels of STEAP2 with a Kd value of at most about 25 nM, at most about 20 nM, at most about 15 nM, at most about 12.5 nM, at most about 10 nM, at most about 7.5 nM, at most about 7 nM, at most about 6.5 nM, at most about 6 nM, at most about 5 nM, at most about 4 nM, at most about 3.5 nM, at most about 3 nM, or at most about 2.5 nM. In some embodiments, provided compounds or pharmaceutically acceptable salt thereof (e.g., immunoconjugates or radioimmunoconjugates) are capable of binding to different cell lines with varying expression levels of STEAP2 with a Kd value of about 15 nM, about 12.5 nM, about 10 nM, about 7.5 nM, about 7 nM, about 6.5 nM, about 6 nM, about 5 nM, about 4 mM, about 3.5 nM, about 3 nM, or about 2.5 nM.

In some embodiments, as further described herein, the compound or pharmaceutically acceptable salt thereof (e.g., immunoconjugate or radioimmunoconjugate) comprises a chelating moiety or a metal complex thereof, which metal complex may comprise a radionuclide. In some such compounds or pharmaceutically acceptable salts thereof, the average ratio or median ratio of the chelating moiety to the STEAP2 targeting moiety (e.g., STEAP2 antibody) is eight or less, seven or less, six or less, five or less, four or less, three or less, two or less, or about one. In some compounds or pharmaceutically acceptable salts thereof, the average ratio or median ratio of the chelating moiety to the STEAP2 targeting moiety (e.g., STEAP2 antibody) is about one.

In some embodiments, after a radioimmunoconjugate is administered to a mammal, the proportion of radiation (of the total amount of radiation that is administered) that is excreted by the intestinal route, the renal route, or both is greater than the proportion of radiation excreted by a comparable mammal that has been administered a reference radioimmunoconjugate. By “reference immunoconjugate” it is meant a known radioimmunoconjugate that differs from a radioimmunoconjugate described herein at least by (1) having a different linker; (2) having a targeting moiety of a different size and/or (3) lacking a targeting moiety. In some embodiments, the reference radioimmunoconjugate is selected from the group consisting of [⁹⁰Y]-ibritumomab tiuxetan (Zevalin (⁹⁰Y)) and [¹¹¹In]-ibritumomab tiuxetan (Zevalin (¹¹¹In)).

In some embodiments, the proportion of radiation excreted by a given route or set of routes) is at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% greater than the proportion of radiation excreted by the same route(s) by a comparable mammal that has been administered a reference radioimmunoconjugate. In some embodiments, the proportion of radiation excreted is at least 1.5-fold, at least 2-fold, at least 2.5-fold, at least 3-fold, at least 3.5-fold, at least 4-fold, at least 4.5-fold, at least 5-fold, at least 6-fold, at least 7-fold, at least 8-fold, at least 9-fold, or at least 10-fold greater than proportion of radiation excreted by a comparable mammal that has been administered a reference radioimmunoconjugate. The extent of excretion can be measured by methods known in the art, e.g., by measuring radioactivity in urine and/or feces and/or by measuring total body radioactivity over a period time. See also, e.g., International Patent Publication WO 2018/024869.

In some embodiments, the extent of excretion is measured at a time period of at least or about 12 hours after administration, at least or about 24 hours after administration, at least or about 2 days after administration, at least or about 3 days after administration, at least or about 4 days after administration, at least or about 5 days after administration, at least or about 6 days after administration, or at least or about 7 days, after administration.

In some embodiments, after a compound or pharmaceutically acceptable salt thereof (e.g., immunoconjugate or radioimmunoconjugate) has been administered to a mammal, the compound (e.g., immunoconjugate or radioimmunoconjugate) exhibits decreased off-target binding effects (e.g., toxicities) as compared to a reference compound (e.g., reference conjugate, e.g., a reference immunoconjugate such as a reference radioimmunoconjugate). In some embodiments, this decreased off-target binding effect is a feature of a compound (e.g., immunoconjugate or radioimmunoconjugate) that also exhibits a greater excretion rate as described herein.

### Targeting Moieties

Targeting moieties include any molecule or any part of a molecule that is capable of binding (e.g., capable of specifically binding, specifically binds to, etc.) to a given target, e.g., STEAP2. In some embodiments, the targeting moiety comprises a protein or polypeptide. In some embodiments, the targeting moiety is selected from the group consisting of antibodies or antigen binding fragments thereof, nanobodies, affibodies, and consensus sequences from Fibronectin type III domains (e.g., Centyrins or Adnectins). In some embodiments, a moiety is both a targeting and a therapeutic moiety, i.e., the moiety is capable of binding to a given target and also confers a therapeutic benefit.

In some embodiments, the targeting moiety has a molecular weight of at least 50 kDa, at least 75 kDa, at least 100 kDa, at least 125 kDa, at least 150 kDa, at least 175 kDa, at least 200 kDa, at least 225 kDa, at least 250 kDa, at least 275 kDa, or at least 300 kDa.

In some embodiments, the targeting moiety specifically binds to and inhibits STEAP2.

By “inhibits,” it is meant that the targeting moiety at least partially inhibits one or more functions of STEAP2. In some embodiments, the targeting moiety impairs signaling downstream of STEAP2, e.g., results in the suppressed growth of tumor cells with varying expression levels of STEAP2.

### STEAP2 Antibodies or Antigen-Binding Fragments Thereof

The disclosure provides antibodies or antigen-binding fragments (including Fv, Fab, Fab′, F(ab′)2, Fab′-SH, diabodies, triabodies, tetrabodies, linear antibodies, single-chain antibody molecules (e.g., scFv)), that bind to STEAP2, e.g., bind specifically to STEAP2, that are for use with (e.g., comprised within) the Compound disclosed herein.

Accordingly, the present disclosure provides Compounds (including pharmaceutically acceptable salts thereof) comprising an antibody or antigen-binding fragment thereof that binds specifically to STEAP2. Such an antibody (or antigen-binding fragment) comprised within the Compound may also be referred to as an anti-STEAP2 antibody or antigen-binding fragment thereof. Where no reference to the specific antigen is provided in connection with an antibody, that should be taken to mean that that antibody or antigen-binding fragment binds to STEAP2, unless it is otherwise obvious that the particular antibody being described is specific for an alternative antigen.

STEAP2 is a member of the STEAP family and encodes a multi-pass membrane protein that localizes to the Golgi complex, the plasma membrane, and the vesicular tubular structures in the cytosol. STEAP2 is understood to be expressed on the surface of antigen-presenting cells for interactions with ligands of immune cells. STEAP2 is also known as UNQ6507/PRO23203, STMP, IPCA1, PUMPCn, STAMP1 or PCANAP1, LOC261729, metalloreductase STEAP2, OTTHUMP00000067572, OTTHUMP00000067573, OTTHUMP00000196964, prostate cancer associated protein 1, prostate cancer-associated protein 1, SixTransMembrane Protein of Prostate 1, protein upregulated in metastatic prostate cancer, six transmembrane epithelial antigen of prostate 2, six-transmembrane epithelial antigen of prostate 2, and any grammatical equivalents.

Aside from the disclosure identifying STEAP2 as a tumor-targeting antigen (TAA) in an array of cancers, the inventors have developed a highly innovative approach for generating STEAP2-specific antibodies. The STEAP2 protein has been largely unstudied because its multi-transmembrane domains pose a significant challenge to antibody development. Without being bound by theory, this may be due to STEAP2's limited extracellular loops, which display near total conservation across species and high homology with other STEAP family members. Consequently, there is a lack of STEAP2-specific commercial antibodies. However, the inventors devised an innovative antigen design strategy to isolate and develop STEAP2-specific antibodies. The approach is characterized in further detail in the Examples.

In some embodiments, the antibody or antigen-binding fragment thereof comprises a heavy chain CDR1 (HCDR1), a heavy chain CDR2 (HCDR2), a heavy chain CDR3 (HCDR3), a light chain CDR1 (LCDR1), a light chain CDR2 (LCDR1) and a light chain CDR3 (LCDR3).

In some embodiments, the antibody or antigen-binding fragment thereof comprises a heavy chain variable domain comprising heavy chain CDR1 (HCDR1), a heavy chain CDR2 (HCDR2), a heavy chain CDR3 (HCDR3), and a light chain variable domain comprising a light chain CDR1 (LCDR1), a light chain CDR2 (LCDR1) and a light chain CDR3 (LCDR3).

In some embodiments, the antibody comprises a full-length heavy chain and a full-length light chain.

Exemplary anti-STEAP2 antibodies and antigen-binding fragments thereof are disclosed in Table 1.

TABLE 1

| | Sequence | Sequence | |
|---|---|---|---|
| Ab | identifier | feature | Sequence |
| 40A3(LO14) | SEQ ID NO: 1 | HCDR1 | RNSAVWN |
| 40A3(LO14) | SEQ ID NO: 2 | HCDR2 | RTYYRSKWYNDYAPSVKS |
| 40A3(LO14) | SEQ ID NO: 3 | HCDR3 | GLRQNQFYYYMDV |
| 40A3(LO14) | SEQ ID NO: 4 | VH | QVQLQQSGPGLVKPSQTLSLTCAIS |
| | | | GDSVSRNSAVWNWIRQSPSRGLEW |
| | | | LGRTYYRSKWYNDYAPSVKSRITIN |
| | | | PDTSKNQFSLQLNSVTPEDTAVYYC |
| | | | ARGLRQNQFYYYMDVWGKGTTVT |
| | | | VSS |
| 40A3(LO14) | SEQ ID NO: 5 | Heavy Chain | QVQLQQSGPGLVKPSQTLSLTCAIS |
| | | | GDSVSRNSAVWNWIRQSPSRGLEW |
| | | | LGRTYYRSKWYNDYAPSVKSRITIN |
| | | | PDTSKNQFSLQLNSVTPEDTAVYYC |
| | | | ARGLRQNQFYYYMDVWGKGTTVT |
| | | | VSSASTKGPSVFPLAPSSKSTSGGTA |
| | | | ALGCLVKDYFPEPVTVSWNSGALT |
| | | | SGVHTFPAVLQSSGLYSLSSVVTVP |
| | | | SSSLGTQTYICNVNHKPSNTKVDKR |
| | | | VEPKSCDKTHTCPPCPAPEFEGGPS |
| | | | VFLFPPKPKDTLMISRTPEVTCVVV |
| | | | DVSHEDPEVKFNWYVDGVEVHNA |
| | | | KTKPREEQYNSTYRVVSVLTVLHQ |
| | | | DWLNGKEYKCKVSNKALPASIEKTI |
| | | | SKAKGQPREPQVYTLPPSREEMTKN |
| | | | QVSLTCLVKGFYPSDIAVEWESNGQ |
| | | | PENNYKTTPPVLDSDGSFFLYSKLT |
| | | | VDKSRWQQGNVFSCSVMHEALHN |
| | | | HYTQKSLSLSPGK |
| 40A3(LO14) | SEQ ID NO: 6 | LCDR1 | RASQSVASNLA |
| 40A3(LO14) | SEQ ID NO: 7 | LCDR2 | GASTRAT |
| 40A3(LO14) | SEQ ID NO: 8 | LCDR3 | QQYNNWPFT |
| 40A3(LO14) | SEQ ID NO: 9 | VL | EIVMTQSPATLSVSPGERATLSCRAS |
| | | | QSVASNLAWYQQKPGQAPRLLIYG |
| | | | ASTRATGIPARFSGSGSGTEFTLTISS |
| | | | LQSEDFAVYYCQQYNNWPFTFGPG |
| | | | TKVDIK |
| 40A3(LO14) | SEQ ID NO: 10 | Light Chain | EIVMTQSPATLSVSPGERATLSCRAS |
| | | | QSVASNLAWYQQKPGQAPRLLIYG |
| | | | ASTRATGIPARFSGSGSGTEFTLTISS |
| | | | LQSEDFAVYYCQQYNNWPFTFGPG |
| | | | TKVDIKRTVAAPSVFIFPPSDEQLKS |
| | | | GTASVVCLLNNFYPREAKVQWKVD |
| | | | NALQSGNSQESVTEQDSKDSTYSLS |
| | | | STLTLSKADYEKHKVYACEVTHQG |
| | | | LSSPVTKSFNRGEC |
| 40A3(LO11) | SEQ ID NO: 11 | HCDR1 | RNSAVWN |
| 40A3(LO11) | SEQ ID NO: 12 | HCDR2 | RTYYRSKWYNDYAPSVKS |
| 40A3(LO11) | SEQ ID NO: 13 | HCDR3 | GLLQNQFYYYMDV |
| 40A3(LO11) | SEQ ID NO: 14 | VH | QVQLQQSGPGLVKPSQTLSLTCAIS |
| | | | GDSVSRNSAVWNWIRQSPSRGLEW |
| | | | LGRTYYRSKWYNDYAPSVKSRITIN |
| | | | PDTSKNQFSLQLNSVTPEDTAVYYC |
| | | | ARGLLQNQFYYYMDVWGKGTTVT |
| | | | VSS |
| 40A3(LO11) | SEQ ID NO: 15 | Heavy Chain | QVQLQQSGPGLVKPSQTLSLTCAIS |
| | | | GDSVSRNSAVWNWIRQSPSRGLEW |
| | | | LGRTYYRSKWYNDYAPSVKSRITIN |
| | | | PDTSKNQFSLQLNSVTPEDTAVYYC |
| | | | ARGLLQNQFYYYMDVWGKGTTVT |
| | | | VSSASTKGPSVFPLAPSSKSTSGGTA |
| | | | ALGCLVKDYFPEPVTVSWNSGALT |
| | | | SGVHTFPAVLQSSGLYSLSSVVTVP |
| | | | SSSLGTQTYICNVNHKPSNTKVDKR |
| | | | VEPKSCDKTHTCPPCPAPEFEGGPS |
| | | | VFLFPPKPKDTLMISRTPEVTCVVV |
| | | | DVSHEDPEVKFNWYVDGVEVHNA |
| | | | KTKPREEQYNSTYRVVSVLTVLHQ |
| | | | DWLNGKEYKCKVSNKALPASIEKTI |
| | | | SKAKGQPREPQVYTLPPSREEMTKN |
| | | | QVSLTCLVKGFYPSDIAVEWESNGQ |
| | | | PENNYKTTPPVLDSDGSFFLYSKLT |
| | | | VDKSRWQQGNVFSCSVMHEALHN |
| | | | HYTQKSLSLSPGK |
| 40A3(LO11) | SEQ ID NO: 16 | LCDR1 | RASQSVASNLA |
| 40A3(LO11) | SEQ ID NO: 17 | LCDR2 | GASTRAT |
| 40A3(LO11) | SEQ ID NO: 18 | LCDR3 | QQYNNWPFT |
| 40A3(LO11) | SEQ ID NO: 19 | VL | EIVMTQSPATLSVSPGERATLSCRAS |
| | | | QSVASNLAWYQQKPGQAPRLLIYG |
| | | | ASTRATGIPARFSGSGSGTEFTLTISS |
| | | | LQSEDFAVYYCQQYNNWPFTFGPG |
| | | | TKVDIK |
| 40A3(LO11) | SEQ ID NO: 20 | Light Chain | EIVMTQSPATLSVSPGERATLSCRAS |
| | | | QSVASNLAWYQQKPGQAPRLLIYG |
| | | | ASTRATGIPARFSGSGSGTEFTLTISS |
| | | | LQSEDFAVYYCQQYNNWPFTFGPG |
| | | | TKVDIKRTVAAPSVFIFPPSDEQLKS |
| | | | GTASVVCLLNNFYPREAKVQWKVD |
| | | | NALQSGNSQESVTEQDSKDSTYSLS |
| | | | STLTLSKADYEKHKVYACEVTHQG |
| | | | LSSPVTKSFNRGEC |
| 40A3 | SEQ ID NO: 21 | HCDR1 | RNSAVWN |
| 40A3 | SEQ ID NO: 22 | HCDR2 | RTYYRSKWYNDYAVSVKS |
| 40A3 | SEQ ID NO: 23 | HCDR3 | GLLQNNFYYYMDV |
| 40A3 | SEQ ID NO: 24 | VH | QVQLQQSGPGLVKPSQTLSLTCAIS |
| | | | GDSVSRNSAVWNWIRQSPSRGLEW |
| | | | LGRTYYRSKWYNDYAVSVKSRITIN |
| | | | PDTSKNQFSLQVNSVTPEDTAVYYC |
| | | | ARGLLQNNFYYYMDVWGKGTTVT |
| | | | VSS |
| 40A3 | SEQ ID NO: 25 | LCDR1 | RASQSVNSNLA |
| 40A3 | SEQ ID NO: 26 | LCDR2 | GASTRAT |
| 40A3 | SEQ ID NO: 27 | LCDR3 | QQYNNWPFT |
| 40A3 | SEQ ID NO: 28 | VL | EIVMTQSPATLSVSPGERATLSCRAS |
| | | | QSVNSNLAWYQQKPGQAPRLLIYG |
| | | | ASTRATGIPARFSGSGSGTEFTLTISS |
| | | | LQSEDFAVYYCQQYNNWPFTFGPG |
| | | | TKVDIK |
| 40A3(LO7) | SEQ ID NO: 31 | HCDR1 | RNSAVWN |
| 40A3(LO7) | SEQ ID NO: 32 | HCDR2 | RTYYRSKWYNDYAVSVKS |
| 40A3(LO7) | SEQ ID NO: 33 | HCDR3 | GLLQNQFYYYMDV |
| 40A3(LO7) | SEQ ID NO: 34 | VH | QVQLQQSGPGLVKPSQTLSLTCAIS |
| | | | GDSVSRNSAVWNWIRQSPSRGLEW |
| | | | LGRTYYRSKWYNDYAVSVKSRITIN |
| | | | PDTSKNQFSLQLNSVTPEDTAVYYC |
| | | | ARGLLQNQFYYYMDVWGKGTTVT |
| | | | VSS |
| 40A3(LO7) | SEQ ID NO: 35 | LCDR1 | RASQSVSSNLA |
| 40A3(LO7) | SEQ ID NO: 36 | LCDR2 | GASTRAT |
| 40A3(LO7) | SEQ ID NO: 37 | LCDR3 | QQYNNWPFT |
| 40A3(LO7) | SEQ ID NO: 38 | VL | EIVMTQSPATLSVSPGERATLSCRAS |
| | | | QSVSSNLAWYQQKPGQAPRLLIYG |
| | | | ASTRATGIPARFSGSGSGTEFTLTISS |
| | | | LQSEDFAVYYCQQYNNWPFTFGPG |
| | | | TKVDIK |

### Variable Domains

In some embodiments, the antibody or antigen-binding fragment comprised within the Compound or pharmaceutically acceptable salt thereof comprises a VH domain comprising (or alternatively, consisting of), any of one of the VH domains disclosed in Table 1.

In some embodiments, the antibody or antigen-binding fragment comprised within the Compound or pharmaceutically acceptable salt thereof comprises a VL domain comprising (or alternatively, consisting of), any of one of the VL domains disclosed in Table 1.

In some embodiments, the antibody or antigen-binding fragment comprised within the Compound or pharmaceutically acceptable salt thereof comprises a VH domain comprising (or alternatively, consisting of), any of one of the VH domains disclosed in Table 1, wherein no more than 1, 2, 3, 4, 5, 6, 7, 8, 9, 10, 11, 12, 13, 14, 15, 16, 17, 18, 19 or 20 amino acids in the framework regions have been mutated (a mutation may variably be an amino acid substitution, deletion or addition).

In some embodiments, the antibody or antigen-binding fragment comprised within the Compound or pharmaceutically acceptable salt thereof comprises a VL domain comprising (or alternatively, consisting of), any of one of the VL domains disclosed in Table 1, wherein no more than 1, 2, 3, 4, 5, 6, 7, 8, 9, 10, 11, 12, 13, 14, 15, 16, 17, 18, 19 or 20 amino acids in the framework regions have been mutated (a mutation may variably be an amino acid substitution, deletion or addition).

In some embodiments, the antibody or antigen-binding fragment thereof comprised within the Compound or pharmaceutically acceptable salt thereof comprises a VH domain having at least 80%, 85%, 90% or 95% sequence identity to the amino acid sequence of a VH domain disclosed in Table 1.

In some embodiments, the antibody or antigen-binding fragment thereof comprised within the Compound or pharmaceutically acceptable salt thereof comprises a VL domain having at least 80%, 85%, 90% or 95% sequence identity to the amino acid sequence of a VL domain disclosed in Table 1.

In some embodiments, the antibody or antigen-binding fragment thereof comprised within the Compound or pharmaceutically acceptable salt thereof comprises a VH domain having (a) at least 80%, 85%, 90% or 95% sequence identity to the amino acid sequence of SEQ ID NO:4; (b) at least 80%, 85%, 90% or 95% sequence identity to the amino acid sequence of SEQ ID NO:14; (c) at least 80%, 85%, 90% or 95% sequence identity to the amino acid sequence of SEQ ID NO:24; or (d) at least 80%, 85%, 90% or 95% sequence identity to the amino acid sequence of SEQ ID NO:34. In some embodiments, the antibody or antigen-binding fragment thereof comprises a VH domain having (a) at least 95% sequence identity to the amino acid sequence of SEQ ID NO:4; (b) at least 95% sequence identity to the amino acid sequence of SEQ ID NO:14; (c) at least 95% sequence identity to the amino acid sequence of SEQ ID NO:24; or (d) at least 95% sequence identity to the amino acid sequence of SEQ ID NO:34.

In some embodiments, the antibody or antigen-binding fragment thereof comprised within the Compound or pharmaceutically acceptable salt thereof comprises a VL domain having (a) at least 80%, 85%, 90% or 95% sequence identity to the amino acid sequence of SEQ ID NO:9; (b) at least 80%, 85%, 90% or 95% sequence identity to the amino acid sequence of SEQ ID NO:19; (c) at least 80%, 85%, 90% or 95% sequence identity to the amino acid sequence of SEQ ID NO:28; or (d) at least 80%, 85%, 90% or 95% sequence identity to the amino acid sequence of SEQ ID NO:38. In some embodiments, the antibody or antigen-binding fragment thereof comprises a VL domain having (a) at least 95% sequence identity to the amino acid sequence of SEQ ID NO:9; (b) at least 95% sequence identity to the amino acid sequence of SEQ ID NO:19; (c) at least 95% sequence identity to the amino acid sequence of SEQ ID NO:28; or (d) at least 95% sequence identity to the amino acid sequence of SEQ ID NO:38.

In some embodiments, the antibody or antigen-binding fragment thereof comprised within the Compound or pharmaceutically acceptable salt thereof comprises a VH domain (a) having at least 80%, 85%, 90% or 95% sequence identity to the amino acid sequence of SEQ ID NO:4 and a VL domain having at least 80%, 85%, 90% or 95% sequence identity to the amino acid sequence of SEQ ID NO:9; (b) a VH domain having at least 80%, 85%, 90% or 95% sequence identity to the amino acid sequence of SEQ ID NO:14 and a VL domain having at least 80%, 85%, 90% or 95% sequence identity to the amino acid sequence of SEQ ID NO:19; (c) a VH domain having at least 80%, 85%, 90% or 95% sequence identity to the amino acid sequence of SEQ ID NO:24 and a VL domain having at least 80%, 85%, 90% or 95% sequence identity to the amino acid sequence of SEQ ID NO:28; or (d) a VH domain having at least 80%, 85%, 90% or 95% sequence identity to the amino acid sequence of SEQ ID NO:34 and a VL domain having at least 80%, 85%, 90% or 95% sequence identity to the amino acid sequence of SEQ ID NO:38.

In some embodiments, the antibody or antigen-binding fragment thereof comprised within the Compound or pharmaceutically acceptable salt thereof comprises a VH domain having (a) at least 95% sequence identity to the amino acid sequence of SEQ ID NO:4 and a VL domain having at least 95% sequence identity to the amino acid sequence of SEQ ID NO:9; (b) a VH domain having at least 95% sequence identity to the amino acid sequence of SEQ ID NO:14 and a VL domain having at least 95% sequence identity to the amino acid sequence of SEQ ID NO:19; (c) a VH domain having at least 95% sequence identity to the amino acid sequence of SEQ ID NO:24 and a VL domain having at least 95% sequence identity to the amino acid sequence of SEQ ID NO:28; or (d) a VH domain having at least 95% sequence identity to the amino acid sequence of SEQ ID NO:34 and a VL domain having at least 95% sequence identity to the amino acid sequence of SEQ ID NO:38.

In some embodiments, the antibody or antigen-binding fragment thereof comprised within the Compound or pharmaceutically acceptable salt thereof comprises a VH domain having at least 95% sequence identity to the amino acid sequence of SEQ ID NO:4 and a VL domain having at least 95% sequence identity to the amino acid sequence of SEQ ID NO:9. In some embodiments, the antibody or antigen-binding fragment thereof comprised within the Compound or pharmaceutically acceptable salt thereof comprises a VH domain having the amino acid sequence of SEQ ID NO:4 and a VL domain having the amino acid sequence of SEQ ID NO:9.

In some embodiments, the antibody or antigen-binding fragment thereof comprised within the Compound or pharmaceutically acceptable salt thereof comprises a VH domain having at least 95% sequence identity to the amino acid sequence of SEQ ID NO:14 and a VL domain having at least 95% sequence identity to the amino acid sequence of SEQ ID NO:19. In some embodiments, the antibody or antigen-binding fragment thereof comprised within the Compound or pharmaceutically acceptable salt thereof comprises a VH domain having the amino acid sequence of SEQ ID NO:14 and a VL domain having the amino acid sequence of SEQ ID NO:19.

In some embodiments, the antibody or antigen-binding fragment thereof comprised within the Compound or pharmaceutically acceptable salt thereof comprises a VH domain having at least 95% sequence identity to the amino acid sequence of SEQ ID NO:24 and a VL domain having at least 95% sequence identity to the amino acid sequence of SEQ ID NO:28. In some embodiments, the antibody or antigen-binding fragment thereof comprised within the Compound or pharmaceutically acceptable salt thereof comprises a VH domain having the amino acid sequence of SEQ ID NO:24 and a VL domain having the amino acid sequence of SEQ ID NO:28.

In some embodiments, the antibody or antigen-binding fragment thereof comprised within the Compound or pharmaceutically acceptable salt thereof comprises a VH domain having at least 95% sequence identity to the amino acid sequence of SEQ ID NO:34 and a VL domain having at least 95% sequence identity to the amino acid sequence of SEQ ID NO:38. In some embodiments, the antibody or antigen-binding fragment thereof comprised within the Compound or pharmaceutically acceptable salt thereof comprises a VH domain having the amino acid sequence of SEQ ID NO:34 and a VL domain having the amino acid sequence of SEQ ID NO:38.

### CDRs

In some embodiments, the antibody or antigen-binding fragment comprised within the compound of Formula I (i.e., comprised within the Compound or pharmaceutically acceptable salt thereof) comprises any of one, two or three of the HCDRs disclosed in Table 1.

In some embodiments, the antibody or antigen-binding fragment comprised within the Compound or pharmaceutically acceptable salt thereof comprises HCDR1 comprising, having or consisting of the sequence of SEQ ID NO:1, 11, 21 or 31, HCDR2 comprising, having or consisting of the sequence of SEQ ID NO:2, 12, 22 or 32 and HCDR3 comprising, having or consisting of the sequence of SEQ ID NO:3, 13, 23 or 33.

In some embodiments, the antibody or antigen-binding fragment comprised within the Compound or pharmaceutically acceptable salt thereof comprises (a) HCDR1 comprising, having or consisting of the sequence of SEQ ID NO:1, HCDR2 comprising, having or consisting of the sequence of SEQ ID NO:2 and HCDR3 comprising, having or consisting of the sequence of SEQ ID NO:3; (b) HCDR1 comprising, having or consisting of the sequence of SEQ ID NO:12, HCDR2 comprising, having or consisting of the sequence of SEQ ID NO:13 and HCDR3 comprising, having or consisting of the sequence of SEQ ID NO:14; (c) HCDR1 comprising, having or consisting of the sequence of SEQ ID NO:21, HCDR2 comprising, having or consisting of the sequence of SEQ ID NO:22 and HCDR3 comprising, having or consisting of the sequence of SEQ ID NO:23; or (d) HCDR1 comprising, having or consisting of the sequence of SEQ ID NO:31, HCDR2 comprising, having or consisting of the sequence of SEQ ID NO:32 and HCDR3 comprising, having or consisting of the sequence of SEQ ID NO:33.

In some embodiments, the antibody or antigen-binding fragment comprised within the Compound or pharmaceutically acceptable salt thereof comprises any of one, two or three of the LCDRs disclosed in Table 1.

In some embodiments, the antibody or antigen-binding fragment comprised within the Compound or pharmaceutically acceptable salt thereof comprises LCDR1 comprising, having or consisting of the sequence of SEQ ID NO:6, 16, 25 or 35, LCDR2 comprising, having or consisting of the sequence of SEQ ID NO:7, 17, 26 or 36 and LCDR3 comprising, having or consisting of the sequence of SEQ ID NO:8, 18, 27 or 37.

In some embodiments, the antibody or antigen-binding fragment comprised within the Compound or pharmaceutically acceptable salt thereof comprises (a) LCDR1 comprising, having or consisting of the sequence of SEQ ID NO:6, LCDR2 comprising, having or consisting of the sequence of SEQ ID NO:7 and LCDR3 comprising, having or consisting of the sequence of SEQ ID NO:8; (b) LCDR1 comprising, having or consisting of the sequence of SEQ ID NO:16, LCDR2 comprising, having or consisting of the sequence of SEQ ID NO:17 and LCDR3 comprising, having or consisting of the sequence of SEQ ID NO:18; (c) LCDR1 comprising, having or consisting of the sequence of SEQ ID NO:25, LCDR2 comprising, having or consisting of the sequence of SEQ ID NO:26 and LCDR3 comprising, having or consisting of the sequence of SEQ ID NO:27; (d) LCDR1 comprising, having or consisting of the sequence of SEQ ID NO:35, LCDR2 comprising, having or consisting of the sequence of SEQ ID NO:36 and LCDR3 comprising, having or consisting of the sequence of SEQ ID NO:37, or for each antibody or antigen-binding fragment, a functional variant thereof.

In some embodiments, the antibody or antigen-binding fragment comprised within the Compound or pharmaceutically acceptable salt thereof comprises a) HCDR1 comprising, having or consisting of the sequence of SEQ ID NO:1, HCDR2 comprising, having or consisting of the sequence of SEQ ID NO:2 and HCDR3 comprising, having or consisting of the sequence of SEQ ID NO:3 and LCDR1 comprising, having or consisting of the sequence of SEQ ID NO:6, LCDR2 comprising, having or consisting of the sequence of SEQ ID NO:7 and LCDR3 comprising, having or consisting of the sequence of SEQ ID NO:8; (b) HCDR1 comprising, having or consisting of the sequence of SEQ ID NO:11, HCDR2 comprising, having or consisting of the sequence of SEQ ID NO:12 and HCDR3 comprising, having or consisting of the sequence of SEQ ID NO:13 and LCDR1 comprising, having or consisting of the sequence of SEQ ID NO:16, LCDR2 comprising, having or consisting of the sequence of SEQ ID NO:17 and LCDR3 comprising, having or consisting of the sequence of SEQ ID NO:18; (c) HCDR1 comprising, having or consisting of the sequence of SEQ ID NO:21, HCDR2 comprising, having or consisting of the sequence of SEQ ID NO:22 and HCDR3 comprising, having or consisting of the sequence of SEQ ID NO:23 and LCDR1 comprising, having or consisting of the sequence of SEQ ID NO:25, LCDR2 comprising, having or consisting of the sequence of SEQ ID NO:26 and LCDR3 comprising, having or consisting of the sequence of SEQ ID NO:27; or (d) HCDR1 comprising, having or consisting of the sequence of SEQ ID NO:31, HCDR2 comprising, having or consisting of the sequence of SEQ ID NO:32 and HCDR3 comprising, having or consisting of the sequence of SEQ ID NO:33 and LCDR1 comprising, having or consisting of the sequence of SEQ ID NO:35, LCDR2 comprising, having or consisting of the sequence of SEQ ID NO:36 and LCDR3 comprising, having or consisting of the sequence of SEQ ID NO:37.

In some embodiments, the antibody or antigen-binding fragment comprised within the Compound or pharmaceutically acceptable salt thereof comprises a HCDR1 comprising, having, or consisting of the sequence of SEQ ID NO:1, a HCDR2 comprising, having, or consisting of the sequence of SEQ ID NO:2, a HCDR3 comprising, having, or consisting of the sequence of SEQ ID NO:3, a LCDR1 comprising, having, or consisting of the sequence of SEQ ID NO:6, a LCDR2 comprising, having, or consisting of the sequence of SEQ ID NO:7, and a LCDR3 comprising, having, or consisting of the sequence of SEQ ID NO:8.

In some embodiments, the antibody or antigen-binding fragment comprised within the Compound or pharmaceutically acceptable salt thereof comprises a HCDR1 comprising, having, or consisting of the sequence of SEQ ID NO:11, a HCDR2 comprising, having, or consisting of the sequence of SEQ ID NO:12, a HCDR3 comprising, having, or consisting of the sequence of SEQ ID NO:13, a LCDR1 comprising, having, or consisting of the sequence of SEQ ID NO:16, a LCDR2 comprising, having, or consisting of the sequence of SEQ ID NO:17, and a LCDR3 comprising, having, or consisting of the sequence of SEQ ID NO:18.

In some embodiments, the antibody or antigen-binding fragment comprised within the Compound or pharmaceutically acceptable salt thereof comprises a HCDR1 comprising, having, or consisting of the sequence of SEQ ID NO:21, a HCDR2 comprising, having, or consisting of the sequence of SEQ ID NO:22, a HCDR3 comprising, having, or consisting of the sequence of SEQ ID NO:23, a LCDR1 comprising, having, or consisting of the sequence of SEQ ID NO:25, a LCDR2 comprising, having, or consisting of the sequence of SEQ ID NO:26, and a LCDR3 comprising, having, or consisting of the sequence of SEQ ID NO:27.

In some embodiments, the antibody or antigen-binding fragment comprised within the Compound or pharmaceutically acceptable salt thereof comprises a HCDR1 comprising, having, or consisting of the sequence of SEQ ID NO:31, a HCDR2 comprising, having, or consisting of the sequence of SEQ ID NO:32, a HCDR3 comprising, having, or consisting of the sequence of SEQ ID NO:33, a LCDR1 comprising, having, or consisting of the sequence of SEQ ID NO:35, a LCDR2 comprising, having, or consisting of the sequence of SEQ ID NO:36, and a LCDR3 comprising, having, or consisting of the sequence of SEQ ID NO:37.

### Full Chains

In some embodiments, the antibody or antigen-binding fragment thereof comprised within the Compound or pharmaceutically acceptable salt thereof comprises a heavy chain having (a) at least 80%, 85%, 90% or 95% sequence identity to the amino acid sequence of SEQ ID NO:5; or (b) at least 80%, 85%, 90% or 95% sequence identity to the amino acid sequence of SEQ ID NO:15. In some embodiments, the antibody or antigen-binding fragment thereof comprises a heavy chain having (a) at least 95% sequence identity to the amino acid sequence of SEQ ID NO:5; or (b) at least 95% sequence identity to the amino acid sequence of SEQ ID NO: 15. In some embodiments, the antibody or antigen-binding fragment thereof comprises a heavy chain having the amino acid sequence of SEQ ID NO:5. In some embodiments, the antibody or antigen-binding fragment thereof comprises a heavy chain having the amino acid sequence of SEQ ID NO:15.

In some embodiments, the antibody or antigen-binding fragment thereof comprised within the Compound or pharmaceutically acceptable salt thereof comprises a light chain having (a) at least 80%, 85%, 90% or 95% sequence identity to the amino acid sequence of SEQ ID NO:10; or (b) at least 80%, 85%, 90% or 95% sequence identity to the amino acid sequence of SEQ ID NO:20. In some embodiments, the antibody or antigen-binding fragment thereof comprises a light chain having (a) at least 95% sequence identity to the amino acid sequence of SEQ ID NO:10; or (b) at least 95% sequence identity to the amino acid sequence of SEQ ID NO:20. In some embodiments, the antibody or antigen-binding fragment thereof comprises a light chain having the amino acid sequence of SEQ ID NO:10. In some embodiments, the antibody or antigen-binding fragment thereof comprises a light chain having the amino acid sequence of SEQ ID NO:20.

In some embodiments, the antibody or antigen-binding fragment thereof comprised within the Compound or pharmaceutically acceptable salt thereof comprises a heavy chain (a) having at least 80%, 85%, 90% or 95% sequence identity to the amino acid sequence of SEQ ID NO:5 and a light chain having at least 80%, 85%, 90% or 95% sequence identity to the amino acid sequence of SEQ ID NO:10; or (b) a heavy chain having at least 80%, 85%, 90% or 95% sequence identity to the amino acid sequence of SEQ ID NO:15 and a light chain having at least 80%, 85%, 90% or 95% sequence identity to the amino acid sequence of SEQ ID NO:20. In some embodiments, the antibody or antigen-binding fragment thereof comprises a heavy chain having at least 90% sequence identity to the amino acid sequence of SEQ ID NO:5 and a light chain having at least 90% sequence identity to the amino acid sequence of SEQ ID NO:10. In some embodiments, the antibody or antigen-binding fragment thereof comprises a heavy chain having at least 90% sequence identity to the amino acid sequence of SEQ ID NO:15 and a light chain having at least 90% sequence identity to the amino acid sequence of SEQ ID NO:20.

In some embodiments, the antibody or antigen-binding fragment thereof comprised within the Compound or pharmaceutically acceptable salt thereof comprises a heavy chain having (a) at least 95% sequence identity to the amino acid sequence of SEQ ID NO:5 and a light chain having at least 95% sequence identity to the amino acid sequence of SEQ ID NO:10; or (b) a heavy chain having at least 95% sequence identity to the amino acid sequence of SEQ ID NO:15 and a light chain having at least 95% sequence identity to the amino acid sequence of SEQ ID NO:20. In some embodiments, the antibody or antigen-binding fragment thereof comprises a heavy chain having at least 95% sequence identity to the amino acid sequence of SEQ ID NO:5 and a light chain having at least 95% sequence identity to the amino acid sequence of SEQ ID NO:10. In some embodiments, the antibody or antigen-binding fragment thereof comprises a heavy chain having at least 95% sequence identity to the amino acid sequence of SEQ ID NO:15 and a light chain having at least 95% sequence identity to the amino acid sequence of SEQ ID NO:20.

In some embodiments, the antibody or antigen-binding fragment thereof comprises a heavy chain having the amino acid sequence of SEQ ID NO:5 and a light chain having at the amino acid sequence of SEQ ID NO:10.

In some embodiments, the antibody or antigen-binding fragment thereof comprises a heavy chain having the amino acid sequence of SEQ ID NO:15 and a light chain having the amino acid sequence of SEQ ID NO:20.

In some embodiments, the disclosure also provides for an antibody or antigen-binding fragment thereof comprised within the Compound or pharmaceutically acceptable salt thereof that binds to the same epitope of STEAP2 as an anti-STEAP2 antibody disclosed herein. For example, the disclosure provides an antibody or antigen-binding fragment thereof that binds to the same epitope of STEAP2 as an anti-STEAP2 antibody having a VH domain of SEQ ID NO:4 and a VL domain of SEQ ID NO:9.

The heavy chain and/or light chain of the antibodies described herein may comprise one or more modifications, for example to abrogate or reduce Fc effector functions, promote formation of a heterodimeric antibody molecule, to increase the efficacy of cognate heavy and light chain pairing, and/or to assist with conjugate formation as described in more detail below. The constant region of a heavy chain (CH) and the constant region of a light chain (CL) that has been modified may be referred to as a modified CH region and CL region, respectively.

The antibody molecule may comprise a mutation in the CH region of the heavy chain to reduce or abrogate binding of the antibody molecule to one or more Fcγ receptors, such as FcγRI, FcγRIIa, FcγRIIb, FcγRIII and/or to complement. Such mutations abrogate or reduce Fc effector functions. Mutations for reduce or abrogate binding of antibody molecule to one or more Fcγ receptors and complement are known and include the “triple mutation” or “TM” of L234F/L235E/P331S described for example in Organesyan, V. et al., Structural characterization of human Fc fragment engineered for lack of effector functions, Acta Crystallographica Section D Biological Crystallography. 64(Pt6): 700-704. 2008. Other mutations that are known to modulate antibody effector function are described for example in Wang, X. et al., IgG Fc engineering to modulate antibody effector functions, Protein & Cell. 9(1): 63-73. 2018.

In some embodiments, the antibody or antigen-binding fragment thereof comprised within the Compound or pharmaceutically acceptable salt thereof is a monoclonal antibody, such as a chimeric, humanized or human antibody.

In some embodiments, the antibody is an antigen-binding fragment. In some embodiments, the antigen-binding fragment comprised within the Compound or pharmaceutically acceptable salt thereof is a Fv, Fab, Fab′, scFv, diabody or F(ab′)₂ fragment.

In some embodiments, the antibody comprised within the Compound or pharmaceutically acceptable salt thereof is a full-length antibody.

The antibody or antigen-binding fragment thereof comprised within the Compound or pharmaceutically acceptable salt thereof is capable of binding specifically to STEAP2 as an integral component of a cancer cell (for example, STEAP2 as an integral component of a cell membrane of a cancer cell). As described in the Examples, because of the difficulty associated with developing antibodies for STEAP2, it was hitherto unknown that antibodies with such binding specificity could be developed at all.

In one embodiment, the antibody or antigen-binding fragment thereof comprised within the Compound may bind to an exemplary prostate cancer cell line and patient derived xenografts, including but not limited to LNCaP. For example, the antibody or antigen-binding fragment thereof may bind to a STEAP2 (e.g., to a STEAP2 epitope) of a LNCaP cell line and/or any cancer cell lines (e.g., which may lack an exogenous nucleic acid encoding STEAP2). Suitably, the antibody or antigen-binding fragment thereof described herein may bind to a LNCaP cell line and a CHO cell line (e.g., which may lack an exogenous nucleic acid encoding STEAP2).

The antibody binding affinity can be measured by any suitable method of measuring binding affinity described herein or known to a person of ordinary skill in the arts.

Suitably, the antibody or antigen-binding fragment comprised within the Compound or pharmaceutically acceptable salt thereof binds to STEAP2 molecule with sufficient affinity such that the antibody is useful as a therapeutic agent or a diagnostic reagent in targeting STEAP2.

In one aspect, the antibody or antigen-binding fragment thereof comprised within the Compound or pharmaceutically acceptable salt thereof binds to STEAP2 (preferably a human STEAP2) with a dissociation constant (KD) of ≤1 μM, ≤100 nM, ≤10 nM, ≤7.5 nM, ≤5 nM, ≤4 nM, ≤3 nM, or ≤2 nM. In one aspect, the antibody or antigen-binding fragment thereof binds to STEAP2 (preferably a human STEAP2) with a dissociation constant (KD) of ≤10 nM. In one aspect, the antibody or antigen-binding fragment thereof binds to STEAP2 (preferably a human STEAP2) with a dissociation constant (KD) of ≤7 nM. In one aspect, the antibody or antigen-binding fragment thereof binds to STEAP2 (preferably a human STEAP2) with a dissociation constant (KD) of ≤5 nM. In one aspect, the antibody or antigen-binding fragment thereof binds to STEAP2 (preferably a human STEAP2) with a dissociation constant (KD) of ≤3 nM. In one aspect, the antibody or antigen-binding fragment thereof binds to STEAP2 (preferably a human STEAP2) with a dissociation constant (KD) of ≤1 nM.

In one aspect, the antibody or antigen-binding fragment thereof binds to a STEAP2 (preferably a human STEAP2) with a KD of between about 0.1 nM to about 40 nM, between about 0.5 nM to about 30 nM, between about 1 nM to about 20 nM, or between about 1 nM to about 10 nM.

In one embodiment, the antibody or antigen-binding fragment thereof comprised within the Compound or pharmaceutically acceptable salt thereof binds to a STEAP2 (preferably a human STEAP2) with a binding affinity of between about 1 nM to about 10 nM. In a more preferable embodiment, the antibody or antigen-binding fragment thereof binds to a STEAP2 (preferably a human STEAP2) with a KD of between about 1 nM to about 5 nM.

The binding affinity measurements may be carried out by any suitable assay known in the art. Suitable assays include an affinity assay performable via a KinExA system (e.g., KinExA 3100, KinExA 3200, or KinExA 4000) (Sapidyne Instruments, Idaho), or ForteBio Octet system.

In one embodiment, the extent of binding of an antibody or antigen-binding fragment thereof of the disclosure to an unrelated, non-STEAP2 protein is less than about 10%, 5%, 2% or 1% of the binding of the antibody (or antigen-binding fragment thereof) to STEAP2 (preferably human STEAP2). In one embodiment, the extent of binding of an antibody or antigen-binding fragment thereof of the disclosure to an unrelated, non-STEAP2 protein is less than about 10% of the binding of the antibody (or antigen-binding fragment thereof) to STEAP2 (preferably human STEAP2). In one embodiment, the extent of binding of an antibody or antigen-binding fragment thereof of the disclosure to an unrelated, non-STEAP2 protein is less than about 5% of the binding of the antibody (or antigen-binding fragment thereof) to STEAP2 (preferably human STEAP2). In one embodiment, the extent of binding of an antibody or antigen-binding fragment thereof of the disclosure to an unrelated, non-STEAP2 protein is less than about 2% of the binding of the antibody (or antigen-binding fragment thereof) to STEAP2 (preferably human STEAP2). In one embodiment, the extent of binding of an antibody or antigen-binding fragment thereof of the disclosure to an unrelated, non-STEAP2 protein is less than about 1% of the binding of the antibody (or antigen-binding fragment thereof) to STEAP2 (preferably human STEAP2). Said binding may be measured, e.g., by a radioimmunoassay (RIA), BIACORE® (using recombinant STEAP2 as the analyte and antibody as the ligand, or vice versa), KINEXA®, ForteBio Octet system, or other binding assays known in the art.

A “STEAP2 polypeptide” may comprise the full length polypeptide sequence of STEAP2 (e.g. SEQ ID NO: 29), or may comprise a fragment of STEAP2 of any length of the full length polypeptide sequence of STEAP2 (e.g. comprising a polypeptide sequence of 5%, 15%, 25%, 35%, 45%, 55%, 65%, 75%, 85% or 95% of the full length polypeptide sequence of STEAP2) which comprises an epitope which can bind (e.g. be bound by) an antibody or antigen-binding fragment of the disclosure. The STEAP2 polypeptide may comprise a sequence having 75%, 80%, 85%, 90% or 90% sequence identity to the sequence of SEQ ID NO.: 29. Preferably, the STEAP2 polypeptide comprises the sequence of SEQ ID NO.: 29. SEQ ID NO: 29:

MESISMMGSPKSLSETFLPNGINGIKDARKVTVGVIGSGDFAKSLTIRL
IRCGYHVVIGSRNPKFASEFFPHVVDVTHHEDALTKTNIIFVAIHREHY
TSLWDLRHLLVGKILIDVSNNMRINQYPESNAEYLASLFPDSLIVKGFN
VVSAWALQLGPKDASRQVYICSNNIQARQQVIELARQLNFIPIDLGSLS
SAREIENLPLRLFTLWRGPVVVAISLATFFFLYSFVRDVIHPYARNQQS
DFYKIPIEIVNKTLPIVAITLLSLVYLAGLLAAAYQLYYGTKYRRFPPW
LETWLQCRKQLGLLSFFFAMVHVAYSLCLPMRRSERYLFLNMAYQQVHA
NIENSWNEEEVWRIEMYISFGIMSLGLLSLLAVTSIPSVSNALNWREFS
FIQSTLGYVALLISTFHVLIYGWKRAFEEEYYRFYTPPNFVLALVLPSI
VILGKIILFLPCISRKLKRIKKGWEKSQFLEEGMGGTIPHVSPERVTVM

### Functional Variants

In some embodiments, the antibody (or antigen-binding fragment thereof) comprised within the Compound is a functional variant of an antibody characterized elsewhere herein by reference to sequence characteristics (e.g., the antibodies described in Table 1). For this purpose, an antibody of Table 1 is termed a “reference antibody”).

In one embodiment, the variant antibody (or antigen-binding fragment thereof) comprises at most 2 amino acid differences in one or more of HCDR1, HCDR2, HCDR3, LCDR1, LCDR2 and LCDR3 of the corresponding reference antibody.

In one embodiment, the variant antibody (or antigen-binding fragment thereof) comprises at most 1 amino acid difference in one or more of HCDR1, HCDR2, HCDR3, LCDR1, LCDR2 and LCDR3 of the corresponding reference antibody.

In one embodiment, the variant antibody (or antigen-binding fragment thereof) comprises at most 2 amino acid differences in one or more of HCDR1 compared to SEQ ID NO: 1, HCDR2 compared to SEQ ID NO: 2, HCDR3 compared to SEQ ID NO: 3, LCDR1 compared to SEQ ID NO: 4, LCDR2 compared to SEQ ID NO: 5 and LCDR3 compared to SEQ ID NO: 6 of the corresponding reference antibody.

In one embodiment, the variant antibody (or antigen-binding fragment thereof) comprises at most 1 amino acid difference in one or more of HCDR1 compared to SEQ ID NO: 1, HCDR2 compared to SEQ ID NO: 2, HCDR3 compared to SEQ ID NO: 3, LCDR1 compared to SEQ ID NO: 4, LCDR2 compared to SEQ ID NO: 5 and LCDR3 compared to SEQ ID NO: 6 of the corresponding reference antibody.

In each embodiment, the variant antibody may exhibit the same antigen cross-reactivity as the reference antigen or antigen-binding fragment thereof.

In one embodiment, a variant antibody may have at most 5, 4 or 3 amino acid differences total in the CDRs thereof when compared to a corresponding reference antibody, with the proviso that there is at most 2 (preferably at most 1) amino acid differences per CDR. In one embodiment, a variant antibody has at most 2 (more preferably at most 1) amino acid differences total in the CDRs thereof when compared to a corresponding reference antibody, with the proviso that there is at most 2 amino acid differences per CDR. In one embodiment, a variant antibody has at most 2 (more preferably at most 1) amino acid differences total in the CDRs thereof when compared to a corresponding reference antibody, with the proviso that there is at most 1 amino acid difference per CDR.

The amino acid difference may be an amino acid substitution, insertion or deletion. In one embodiment, the amino acid difference is a conservative amino acid substitution. Conservative substitution refers to the substitution of an amino acid with another within the same general class such as, for example, one acidic amino acid with another acidic amino acid, one basic amino acid with another basic amino acid or one neutral amino acid by another neutral amino acid. What is intended by a conservative amino acid substitution is well known in the art. A table of some well-known substitutable amino acids based on particular characteristics are provided in Table 2 below:

TABLE 2

| Class | Exemplary substitutable amino acids |
|---|---|
| Aliphatic | G, A, V, L, I |
| Hydroxyl or sulphur/ | S, C, U, T, M |
| selenium-containing | |
| Cyclic | P |
| Aromatic | F, Y, W |
| Basic | H, K, R |
| Acidic | D, E, N, Q |

In one embodiment, a variant antibody has the same framework sequences as the exemplary antibodies described herein. In another embodiment, the variant antibody may comprise a framework region having at most 2, or at most 1, amino acid difference, when compared to a corresponding reference antibody framework sequence. Accordingly, each framework region may have at most 2, or at most 1 amino acid difference, when compared to a corresponding reference antibody framework sequence.

In one embodiment, a variant antibody may have at most 5, 4 or 3 amino acid differences total in the framework regions thereof when compared to a corresponding reference antibody, with the proviso that there is at most 2 (or at most 1) amino acid differences per framework region. In one embodiment, a variant antibody has at most 2 (or at most 1) amino acid differences total in the framework regions thereof when compared to a corresponding reference antibody, with the proviso that there is at most 2 amino acid differences per framework region. In one embodiment, a variant antibody has at most 2 (or at most 1) amino acid differences total in the framework regions thereof when compared to a corresponding reference antibody, with the proviso that there is at most 1 amino acid difference per framework region.

Thus, a variant antibody may comprise a variable heavy chain and a variable light chain as described herein, wherein: (i) the heavy chain has at most 14 amino acid differences (at most 2 amino acid differences in each CDR and at most 2 amino acid differences in each framework region) when compared to a reference antibody heavy chain sequence herein; and (ii) the light chain has at most 14 amino acid differences (at most 2 amino acid differences in each CDR and at most 2 amino acid differences in each framework region) when compared to a reference antibody light chain sequence herein, wherein the variant antibody binds to the same target antigen as the reference antibody, and optionally exhibits the same antigen cross-reactivity (or lack thereof) as the reference antibody.

### Competes for Binding

In some embodiments, the disclosure provides a compound or pharmaceutically acceptable salt that comprises an antibody or antigen-binding fragment thereof that competes or cross-competes for binding to STEAP2 with another anti-STEAP2 antibody or antigen-binding fragment thereof disclosed herein.

For example, in some embodiments, the compound or pharmaceutically acceptable salt comprises an antibody or antigen-binding fragment that competes or cross-competes for binding to STEAP2 with an antibody or antigen-binding fragment that comprises HCDR1 comprising, having, or consisting of SEQ ID NO:1, HCDR2 comprising, having, or consisting of SEQ ID NO:2, HCDR3 comprising, having, or consisting of SEQ ID NO:3, LCDR1 comprising, having, or consisting of SEQ ID NO:6, LCDR2 comprising, having, or consisting of SEQ ID NO:7, and LCDR3 comprising, having, or consisting of SEQ ID NO:8. In some embodiments, the compound or pharmaceutically acceptable salt comprises an antibody or antigen-binding fragment that competes or cross-competes for binding to STEAP2 with an antibody or antigen-binding fragment that comprises a VH domain comprising, having, or consisting of SEQ ID NO:4 and a VL domain comprising, having, or consisting of SEQ ID NO:9. In some embodiments, the compound or pharmaceutically acceptable salt comprises an antibody or antigen-binding fragment that competes or cross-competes for binding to STEAP2 with an antibody or antigen-binding fragment that comprises a heavy chain comprising, having, or consisting of SEQ ID NO:5 and a light chain comprising, having, or consisting of SEQ ID NO:10.

In some embodiments, the compound or pharmaceutically acceptable salt comprises an antibody or antigen-binding fragment that competes or cross-competes for binding to STEAP2 with an antibody or antigen-binding fragment that comprises HCDR1 comprising, having, or consisting of SEQ ID NO:11, HCDR2 comprising, having, or consisting of SEQ ID NO:12, HCDR3 comprising, having, or consisting of SEQ ID NO:13, LCDR1 comprising, having, or consisting of SEQ ID NO:16, LCDR2 comprising, having, or consisting of SEQ ID NO:17, and LCDR3 comprising, having, or consisting of SEQ ID NO:18. In some embodiments, the compound or pharmaceutically acceptable salt comprises an antibody or antigen-binding fragment that competes or cross-competes for binding to STEAP2 with an antibody or antigen-binding fragment that comprises a VH domain comprising, having, or consisting of SEQ ID NO:14 and a VL domain comprising, having, or consisting of SEQ ID NO:19. In some embodiments, the compound or pharmaceutically acceptable salt comprises an antibody or antigen-binding fragment that competes or cross-competes for binding to STEAP2 with an antibody or antigen-binding fragment that comprises a heavy chain comprising, having, or consisting of SEQ ID NO:15 and a light chain comprising, having, or consisting of SEQ ID NO:20.

In some embodiments, the compound or pharmaceutically acceptable salt comprises an antibody or antigen-binding fragment that competes or cross-competes for binding to STEAP2 with an antibody or antigen-binding fragment that comprises HCDR1 comprising, having, or consisting of SEQ ID NO:21, HCDR2 comprising, having, or consisting of SEQ ID NO:22, HCDR3 comprising, having, or consisting of SEQ ID NO:23, LCDR1 comprising, having, or consisting of SEQ ID NO:25, LCDR2 comprising, having, or consisting of SEQ ID NO:26, and LCDR3 comprising, having, or consisting of SEQ ID NO:27. In some embodiments, the compound or pharmaceutically acceptable salt comprises an antibody or antigen-binding fragment that competes or cross-competes for binding to STEAP2 with an antibody or antigen-binding fragment that comprises a VH domain comprising, having, or consisting of SEQ ID NO:24 and a VL domain comprising, having, or consisting of SEQ ID NO:28.

In some embodiments, the compound or pharmaceutically acceptable salt comprises an antibody or antigen-binding fragment that competes or cross-competes for binding to STEAP2 with an antibody or antigen-binding fragment that comprises HCDR1 comprising, having, or consisting of SEQ ID NO:31, HCDR2 comprising, having, or consisting of SEQ ID NO:32, HCDR3 comprising, having, or consisting of SEQ ID NO:33, LCDR1 comprising, having, or consisting of SEQ ID NO:35, LCDR2 comprising, having, or consisting of SEQ ID NO:36, and LCDR3 comprising, having, or consisting of SEQ ID NO:37. In some embodiments, the compound or pharmaceutically acceptable salt comprises an antibody or antigen-binding fragment that competes or cross-competes for binding to STEAP2 with an antibody or antigen-binding fragment that comprises a VH domain comprising, having, or consisting of SEQ ID NO:34 and a VL domain comprising, having, or consisting of SEQ ID NO:38.

Competitive binding can be determined any one of a number of well know assays, including for example, solid phase assays such as competition ELISA assays, Dissociation-Enhanced Lanthanide Fluorescent Immunoassays (DELFIA®, Perkin Elmer), and radioligand binding assays. In one embodiment, the skilled person could determine whether an antibody or antigen-binding fragment thereof competes for binding to STEAP2 by using an in vitro competitive binding assay, such as a derivation of the homogeneous time-resolved fluorescence HTRF assay described in example 1 of WO2016/156440, which is hereby incorporated by reference. For example, the skilled person could label an antibody of Table 1 with a donor fluorophore and mix multiple concentrations with fixed concentration samples of acceptor fluorophore labelled-STEAP2 of SEQ ID NO:29 or fluorophore-labelled chimeric STEAP3-2 antigen of SEQ ID NO:30 (“STEAP3-2”). Subsequently, the fluorescence resonance energy transfer between the donor and acceptor fluorophore within each sample can be measured to ascertain binding characteristics. To elucidate competitive binding molecules the skilled person could first mix various concentrations of a test binding molecule with a fixed concentration of the labelled antibody of Table 1. A reduction in the FRET signal when the mixture is incubated with labelled STEAP2 or STEAP3-2 in comparison with a labelled antibody-only positive control would indicate competitive binding to STEAP2. An antibody or antigen-binding fragment thereof may be said to competitively inhibit binding of the reference antibody to a given epitope by at least 90%, at least 80%, at least 70%, at least 60%, or at least 50%.

(STEAP3-2 chimera-STEAP2-derived extracellular
loop sequences underlined)
SEQ ID NO: 30
MSHQPAVATKMPEEMDKPLISLHLVDSDSSLAKVPDEAPKVGILGSGDF
ARSLATRLVGSGFKVVVGSRNPKRTARLFPSAAQVTFQEEAVSSPEVIF
VAVFREHYSSLCSLSDQLAGKILVDVSNPTEQEHLQHRESNAEYLASLF
PTCTVVKAFNVISAWTLQAGPRDGNRQVPICGDQPEAKRAVSEMALAMG
FMPVDMGSLASAWEVEAMPLRLLPAWKVPTLLALGLFVCFYAYSFVRDV
IHPYARNQQSDFYKIPIEIVNKTLPCVAYVLLSLVYLPGVLAAALQLRR
GTKYQRFPDWLDHWLQHRKQIGLLSFFCAALHALYSFCLPLRRSERYLF
LNMAYQQVHANIENSWNEEEVWRIEIYLSLGVLALGTLSLLAVTSLPSI
ANSLNWREFSFVQSSLGFVALVLSTLHTLTYGWKRAFEEEYYRFYTPPN
FTLTLLVPCVVILAKALFLLPCISRRLARIRRGWERESTIKFTLPTDHA
LAEKTSHV

### Chelating Moiety or Metal Complex Thereof

### Chelating Moieties

Examples of suitable chelating moieties include, but are not limited to, DOTA (1,4,7,10-tetraazacyclododecane-1,4,7,10-tetraacetic acid), DOTMA (1R,4R,7R,10R)-α,α,α″, α″′-tetramethyl-1,4,7,10-tetraazacyclododecane-1,4,7,10-tetraacetic acid, DOTAM (1,4,7,10-tetrakis(carbamoylmethyl)-1,4,7,10-tetraazacyclododecane), DOTPA (1,4,7,10-tetraazacyclododecane-1,4,7,10-tetra propionic acid), DO3AM-acetic acid (2-(4,7,10-tris(2-amino-2-oxoethyl)-1,4,7,10-tetraazacyclododecan-1-yl)acetic acid), DOTA-GA anhydride (2,2′,2″-(10-(2,6-dioxotetrahydro-2H-pyran-3-yl)-1,4,7,10-tetraazacyclododecane-1,4,7-triyl)triacetic acid, DOTP (1,4,7,10-tetraazacyclododecane-1,4,7,10-tetra(methylene phosphonic acid)), DOTMP (1,4,6,10-tetraazacyclodecane-1,4,7,10-tetramethylene phosphonic acid, DOTA-4AMP (1,4,7,10-tetraazacyclododecane-1,4,7,10-tetrakis(acetamido-methylenephosphonic acid), CB-TE2A (1,4,8,11-tetraazabicyclo[6.6.2]hexadecane-4,11-diacetic acid), NOTA (1,4,7-triazacyclononane-1,4,7-triacetic acid), NOTP (1,4,7-triazacyclononane-1,4,7-tri(methylene phosphonic acid), TETPA (1,4,8,11-tetraazacyclotetradecane-1,4,8,11-tetrapropionic acid), TETA (1,4,8,11-tetraazacyclotetradecane-1,4,8,11-tetra acetic acid), HEHA (1,4,7,10,13,16-hexaazacyclohexadecane-1,4,7,10,13,16-hexaacetic acid), PEPA (1,4,7,10,13-pentaazacyclopentadecane-N,N′,N″,N″′, N″″-pentaacetic acid), H₄octapa (N,N′-bis(6-carboxy-2-pyridylmethyl)-ethylenediamine-N,N′-diacetic acid), H₂dedpa (1,2-[[6-(carboxy)-pyridin-2-yl]-methylamino]ethane), H₆phospa (N,N′-(methylenephosphonate)-N,N′-[6-(methoxycarbonyl)pyridin-2-yl]-methyl-1,2-diaminoethane), TTHA (triethylenetetramine-N,N,N′,N″,N″′, N″′-hexaacetic acid), DO2P (tetraazacyclododecane dimethanephosphonic acid), HP-D03A (hydroxypropyltetraazacyclododecanetriacetic acid), EDTA (ethylenediaminetetraacetic acid), Deferoxamine, DTPA (diethylenetriaminepentaacetic acid), DTPA-BMA (diethylenetriaminepentaacetic acid-bismethylamide), octadentate-HOPO (octadentate hydroxypyridinones), or porphyrins.

Preferably, the chelating moiety is selected from DOTA (1,4,7,10-tetraazacyclododecane-1,4,7,10-tetraacetic acid), DOTMA (1R,4R,7R,10R)-α,α′,α″,α′″-tetramethyl-1,4,7,10-tetraazacyclododecane-1,4,7,10-tetraacetic acid, DOTAM (1,4,7,10-tetrakis(carbamoylmethyl)-1,4,7,10-tetraazacyclododecane), DO3AM-acetic acid (2-(4,7,10-tris(2-amino-2-oxoethyl)-1,4,7,10-tetraazacyclododecan-1-yl)acetic acid), DOTP (1,4,7,10-tetraazacyclododecane-1,4,7,10-tetra(methylene phosphonic acid)), DOTA-4AMP (1,4,7,10-tetraazacyclododecane-1,4,7,10-tetrakis(acetamido-methylenephosphonic acid), NOTA (1,4,7-triazacyclononane-1,4,7-triacetic acid), and HP-DO3A (10-(2-hydroxypropyl)-1,4,7-tetraazacyclododecane-1,4,7-triacetic acid).

In some embodiments, the chelating moiety is DOTA.

In some embodiments, compounds comprise a metal complex of a chelating moiety. For example, chelating groups may be used in metal chelate combinations with metals, such as manganese, iron, and gadolinium and isotopes (e.g., isotopes in the general energy range of 60 to 10,000 keV), such as any of the radioisotopes and radionuclides discussed herein.

In some embodiments, chelating moieties are useful as detection agents, and compounds comprising such detectable chelating moieties can therefore be used as diagnostic or theranostic agents.

### Radioisotopes and Radionuclides

In some embodiments, the metal complex comprises a radionuclide. Examples of suitable radioisotopes and radionuclides include, but are not limited to, ³H, ¹⁴C, ¹⁵N, ¹⁸F, ³⁵S, ⁴⁴Sc, ⁴⁷Sc, ⁵⁵Co, ⁶⁰Cu, ⁶¹Cu, ⁶²Cu, ⁶⁴Cu, ⁶⁶Ga ⁶⁷Ga, ⁶⁷Cu, ⁶⁸Ga, ⁷⁵Br, ⁷⁶Br, ⁷⁷Br, ⁸²Rb, ⁸⁹Zr, ⁸⁶Y ⁸⁷Y, ⁹⁰Y, ⁹⁷Ru, ⁹⁹Tc, ^{99m}Tc, ¹⁰⁵Rh, ¹⁰⁹Pd, ¹¹¹In, ¹²³I, ¹²⁴I, ¹²⁵I, ¹³¹I, ¹⁴⁹Pm, ¹⁴⁹Tb, ¹⁵³Sm, ¹⁶⁶Ho, ¹⁷⁷Lu, ^{117m}Sn, ¹⁸⁶Re, ¹⁸⁸Re, ¹⁹⁸Au, ¹⁹⁹Au, ²⁰¹Tl, ²⁰³Pb, ²¹¹At, ²¹²Pb, ²¹²Bi, ²¹³Bi, ²²³Ra, ²²⁵Ac, ^{227Th}, and ^{229Th}.

In some embodiments, the metal complex comprises a radionuclide selected from ⁴⁴Sc, ⁴⁷Sc, ⁵⁵Co, ⁶²Cu, ⁶¹Cu, ⁶²Cu, ⁶⁴Cu, ⁶⁷Cu, ⁶⁶Ga ⁶⁷Ga, ⁶⁸Ga, ⁸²Rb, ⁸⁶Y, ⁸⁷Y, ⁸⁹Zr, ⁹⁰Y, ⁹⁷Ru, ⁹⁹Tc, ^{99m}Tc, ¹⁰⁵Rh, ¹⁰⁹Pd, ¹¹¹In, ^{117m}Sn, ¹⁴⁹Pm, ¹⁴⁹Tb, ¹⁵³Sm, ¹⁶⁶Ho, ¹⁷⁷Lu, ¹⁸⁶Re, ¹⁸⁸Re, ¹⁹⁸Au, ¹⁹⁹Au, ²⁰¹Tl, ²⁰³Pb, ²¹¹At ²¹²Pb, ²¹²Bi, ²¹³Bi, ²²³Ra, ²²⁵Ac, ²²⁷Th and ²²⁹Th.

In certain embodiments, the metal complex comprises a radionuclide selected from ⁶⁸Ga, ⁸⁹Zr, ⁹⁰Y¹¹¹n, ¹⁷⁷Lu, and ²²⁵Ac. In certain embodiments, the metal complex comprises a radionuclide of ¹⁷⁷Lu or ²²⁵Ac.

In some embodiments, the radionuclide is an alpha emitter, e.g., Astatine-211 (²¹¹At), Bismuth-212 (²¹²Bi), Bismuth-213 (²¹³Bi), Actinium-225 (²²⁵Ac), Radium-223 (²²³Ra), Lead-212 (²¹²Pb), Thorium-227 (²²⁷Th) or Terbium-149 (¹⁴⁹Tb), or a progeny thereof. In some embodiments, the alpha-emitter is Actinium-225 (²²⁵Ac), or a progeny thereof.

In certain embodiments, the metal complex comprises an alpha emitter of ²²⁵Ac or a progeny thereof.

### Linker

The compounds of this disclosure comprise the structure of Formula I below:

A-L¹-(L²)ₙ-B Formula I
wherein each of the variables is defined in the SUMMARY section above.
Each of the compounds of Formula I comprises a linker moiety as -L¹-(L²)ₙ-, wherein:
L¹ is a bond, C═O, C═S, optionally substituted C₁-C₆ alkyl, optionally substituted C₁-C₆ heteroalkyl, optionally substituted aryl, or optionally substituted heteroaryl;
n is an integer between 1 and 5 (inclusive); and
each L², independently, has the structure:

—X¹-L³Z¹— Formula II
wherein:
X¹ is —C(O)NR¹—*, —NR¹C(O)—*, —C(S)NR¹—*, —NR¹C(S)—*, —OC(O)NR¹—*, —NR¹C(O)O—*, —NR¹C(O)NR¹—*, —CH₂-Ph-C(O)NR¹—*, —NR¹C(O)-Ph-CH₂—*, —CH₂-Ph-NH—C(S)NR¹—*, —NR¹C(S)—NH-Ph-CH₂—*, —O—*, or —NR¹—*; wherein “*” indicates the attachment point to L³, and R¹ is hydrogen, optionally substituted C₁-C₆ alkyl, optionally substituted C₁-C₆ heteroalkyl, or optionally substituted aryl or heteroaryl;
L³ is optionally substituted C₁-C₅₀alkyl or optionally substituted C₁-C₅₀ heteroalkyl (e.g., (CH₂CH₂O)²-20); and
Z¹ is —CH²-#, —C(O)— #, —C(S)— #, —OC(O)— #, —C(O)O— #, —NR²C(O)— #, —C(O)NR²— #, or —NR²— #, wherein “#” indicates the attachment point to B, and R² is hydrogen, optionally substituted C₁-C₆ alkyl, optionally substituted C₁-C₆ heteroalkyl, optionally substituted aryl, or optionally substituted heteroaryl.

As described herein, the phrase “optionally substituted X” (e.g., optionally substituted alkyl) is intended to be equivalent to “X, wherein X is optionally substituted” (e.g., “alkyl, wherein one or more of the carbons of said alkyl is optionally substituted”). It is not intended to mean that the feature “X” (e.g., alkyl) per se is optional. Without limitation, a substituent can be alkyl, alkenyl, alkynyl, cycloalkyl, heteroalkyl, heterocycloalkyl, haloalkyl, aryl, heteroaryl, each of which has the same meaning as commonly used in the field. For example, the term “optionally substituted aryl” means an aryl that may be optionally substituted with one, two, three, or four substituents independently selected from alkyl, alkenyl, alkynyl, cycloalkyl, heteroalkyl, heterocycloalkyl, haloalkyl, aryl, and heteroaryl.

In some embodiments, L¹ is optionally substituted C₁-C₆ alkyl or optionally substituted C₁-C₆ heteroalkyl. In certain embodiments, L¹ is substituted C₁-C₆ alkyl or substituted C₁-C₆ heteroalkyl, the substituent comprising a heteroaryl group (e.g., six-membered nitrogen-containing heteroaryl). In some embodiments, L¹ is C₁-C₆ alky. For example, L¹ is —CH₂CH₂—.

In some embodiments, L¹ is a bond. In some embodiments, L¹ is

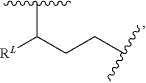

wherein R^{L} is hydrogen or —CO₂H.

In some embodiments, X¹ is-C(O)NR¹—*, —NR¹C(O)—*, or —NR¹—, “*” indicating the attachment point to L³, and R¹ is hydrogen, optionally substituted C₁-C₆ alkyl, optionally substituted C₁-C₆ heteroalkyl, optionally substituted aryl, or optionally substituted heteroaryl. In some embodiments, X¹ is —C(O)NR¹—*, “*” indicating the attachment point to L³, and R¹ is hydrogen.

In some embodiments, L³ is optionally substituted C₁-C₅₀alkyl (e.g., C₃-C₃₀alkyl, C₃-C₂₅ alkyl, C₃-C₂₀ alkyl, C₃-C₁₅ alkyl, C₃-C₁₀ alkyl, C₅-C₃₀alkyl, C₅-C₂₅ alkyl, C₅-C₂₀ alkyl, C₅-C₁₅ alkyl, and C₅-C₁₀ alkyl) or optionally substituted C₁-C₅₀ heteroalkyl (e.g., C₃-C₃₀ heteroalkyl, C₃-C₂₅ heteroalkyl, C₃-C₂₀ heteroalkyl, C₃-C₁₅ heteroalkyl, C₃-C₁₀ heteroalkyl, C₅-C₃₀ heteroalkyl, C₅-C₂₅ heteroalkyl, C₅-C₂₀ heteroalkyl, C₅-C₁₅ heteroalkyl, and C₅-C₁₀ heteroalkyl). An exemplary C₁-C₅₀ heteroalkyl is C₅-C₃₀ polyethylene glycol (e.g., C₅-C₂₅ polyethylene glycol, C₅-C₂₀ polyethylene glycol, C₅-C₁₅ polyethylene glycol). In certain embodiments, L³ is C₅-C₂₅ polyethylene glycol, C₅-C₂₀ polyethylene glycol, or C₅-C₁₅ polyethylene glycol.

In some embodiments, L³ is optionally substituted C₁-C₅₀ heteroalkyl (e.g., C₁-C₄₀ heteroalkyl, C₁-C₃₀ heteroalkyl, C₁-C₂₀ heteroalkyl, C₂-C₁₈ heteroalkyl, C₃-C₁₆ heteroalkyl, C₄-C₁₄ heteroalkyl, C₅-C₁₂ heteroalkyl, C₆-C₁₀ heteroalkyl, C₅-C₁₀ heteroalkyl, C₄ heteroalkyl, C₆ heteroalkyl, C₈ heteroalkyl, C₁₀ heteroalkyl, C₁₂ heteroalkyl, C₁₆ heteroalkyl, C₂₀ heteroalkyl, or C₂₄ heteroalkyl).

In some embodiments, L³ is optionally substituted C₁-C₅₀ heteroalkyl comprising a polyethylene glycol (PEG) moiety comprising 1-20 oxyethylene (—O—CH₂—CH₂—) units, e.g., 2 oxyethylene units (PEG2), 3 oxyethylene units (PEG3), 4 oxyethylene units (PEG4), 5 oxyethylene units (PEG5), 6 oxyethylene units (PEG6), 7 oxyethylene units (PEG7), 8 oxyethylene units (PEG8), 9 oxyethylene units (PEG9), 10 oxyethylene units (PEG10), 12 oxyethylene units (PEG12), 14 oxyethylene units (PEG14), 16 oxyethylene units (PEG16), or 18 oxyethylene units (PEG18).

In certain embodiments, L³ is optionally substituted C₁₋₅₀ heteroalkyl comprising a polyethylene glycol (PEG) moiety comprising 1-20 oxyethylene (—O—CH₂—CH₂—) units or portions thereof. For example, L³ comprises PEG3 as shown below:

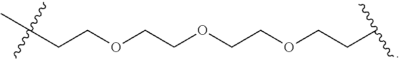 [SMILES: *CCOCCOCCOCC*]

In some embodiments, L³ is (CH₂CH₂O)ₘ(CH₂)_{w}, and m and w are each independently an integer between 0 and 10 (inclusive), and at least one of m and w is not 0.

In some embodiments, L³ is substituted C₁-C₅₀alkyl or substituted C₁-C₅₀ heteroalkyl, the substituent comprising a heteroaryl group (e.g., six-membered nitrogen-containing heteroaryl).

In some embodiments, Z¹ is CH₂, C═O, or NR¹; wherein R¹ is H, optionally substituted C₁-C₆ alkyl, optionally substituted C₁-C₆ heteroalkyl, optionally substituted aryl, or optionally substituted heteroaryl.

In certain embodiments, A-L¹-(L²)ₙ-B can be represented by the following structure:

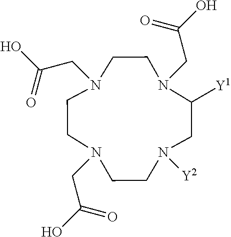

wherein Y¹ is —CH₂OCH₂(L²)ₙ-B, C═O(L²)ₙ-B, or C=S(L²)ₙ-B and Y² is —CH₂CO₂H; or wherein Y¹ is H and Y² is L¹-(L²)ₙ-B.

### Cross-Linking Groups

In some embodiments, compounds (e.g., radioimmunoconjugates) are synthesized using bifunctional chelates that comprise a chelate, a linker, and a cross-linking group. Once the compound (e.g., radioimmunoconjugate) is formed, the cross-linking group may be absent from the compound (e.g., radioimmunoconjugate).

In some embodiments, compounds (e.g., radioimmunoconjugates) comprise a cross-linking group instead of, or in addition to, the targeting moiety (e.g., in some embodiments, B in Formula I comprises a cross-linking group).

A cross-linking group is a reactive group that is able to join two or more molecules by a covalent bond. Cross-linking groups may be used to attach the linker and chelating moiety to a therapeutic or targeting moiety. Cross-linking groups may also be used to attach the linker and chelating moiety to a target in vivo. In some embodiments, the cross-linking group is an amino-reactive, methionine reactive or thiol-reactive cross-linking group, or comprises a sortase recognition sequence (i.e., LPXTG (SEQ ID NO: 39), where X is any amino acid). In some embodiments, the amino-reactive or thiol-reactive cross-linking group comprises an activated ester such as a hydroxysuccinimide ester, 2,3,5,6-tetrafluorophenol ester, 4-nitrophenol ester or an imidate, anhydride, thiol, disulfide, maleimide, azide, alkyne, strained alkyne, strained alkene, halogen, sulfonate, haloacetyl, amine, hydrazide, diazirine, phosphine, tetrazine, isothiocyanate, or oxaziridine. In some embodiments, the sortase recognition sequence may comprise of a terminal glycine-glycine-glycine (GGG) and/or LPTXG amino acid sequence (SEQ ID NO: 40), where X is any amino acid. A person having ordinary skill in the art will understand that the use of cross-linking groups is not limited to the specific constructs disclosed herein, but rather may include other known cross-linking groups.

### Pharmaceutical Compositions

In one aspect, the present disclosure provides pharmaceutical compositions comprising compounds disclosed herein. Such pharmaceutical compositions can be formulated for use in a variety of drug delivery systems. One or more physiologically acceptable excipients or carriers can also be included in a pharmaceutical composition for proper formulation. Non-limiting examples of suitable formulations compatible for use with the present disclosure include those described in Remington's Pharmaceutical Sciences, Mack Publishing Company, Philadelphia, PA, 17^{th} ed., 1985. For a brief review of methods for drug delivery, See, e.g., Langer (Science. 249:1527-1533, 1990).

Pharmaceutical compositions may be formulated for any of a variety of routes of administration discussed herein (See, e.g., the “Administration and Dosage” subsection herein), Sustained release administration is contemplated, by such means as depot injections or erodible implants or components. Thus, the present disclosure provides pharmaceutical compositions that include agents disclosed herein (e.g., radioimmunoconjugates) dissolved or suspended in an acceptable carrier, preferably an aqueous carrier, e.g., water, buffered water, saline, or PBS, among others. In some embodiments, pharmaceutical compositions contain pharmaceutically acceptable auxiliary substances to approximate physiological conditions, such as pH adjusting and buffering agents, tonicity adjusting agents, wetting agents, or detergents, among others. In some embodiments, pharmaceutical compositions are formulated for oral delivery and may optionally contain inert ingredients such as binders or fillers for the formulation of a unit dosage form, such as a tablet or a capsule. In some embodiments, pharmaceutical compositions are formulated for local administration and may optionally contain inert ingredients such as solvents or emulsifiers for the formulation of a cream, an ointment, a gel, a paste, or an eye drop.

In some embodiments, provided pharmaceutical compositions are sterilized by conventional sterilization techniques, e.g., may be sterile filtered. Resulting aqueous solutions may be packaged for use as is, or lyophilized. Lyophilized preparations can be, for example, combined with a sterile aqueous carrier prior to administration. The pH of preparations typically will be between 3 and 11, more preferably between 5 and 9 or between 6 and 8, and most preferably between 6 and 7, such as 6 to 6.5. Resulting compositions in solid form may be packaged, for example, in multiple single dose units, each containing a fixed amount of the above-mentioned agent or agents, such as in a sealed package of tablets or capsules. Pharmaceutical compositions in solid form can also be packaged in a container for a flexible quantity, such as in a squeezable tube designed for a topically applicable cream or ointment.

### Methods of Treatment

In one aspect, the present disclosure provides methods of treatment comprising administering to a subject in need thereof a compound (e.g., radioimmunoconjugate) as disclosed herein.

### Subjects

In some disclosed methods, a therapy (e.g., comprising a therapeutic agent) is administered to a subject. In some embodiments, the subject is a mammal, e.g., a human.

In some embodiments, the subject has cancer or is at risk of developing cancer. For example, the subject may have been diagnosed with cancer. For example, the cancer may be a primary cancer or a metastatic cancer. Subjects may have any stage of cancer, e.g., stage I, stage II, stage III, or stage IV with or without lymph node involvement and with or without metastases.

Provided compounds (e.g., radioimmunoconjugates) and compositions may prevent or reduce further growth of the cancer and/or otherwise ameliorate the cancer (e.g., prevent or reduce metastases). In some embodiments, the subject does not have cancer but has been determined to be at risk of developing cancer, e.g., because of the presence of one or more risk factors such as environmental exposure, presence of one or more genetic mutations or variants, family history, etc. In some embodiments, the subject has not been diagnosed with cancer.

In some embodiments, the cancer is any cancer that comprises cells expressing STEAP2. In certain embodiments, the cancer is lung cancer, colorectal cancer, pancreatic cancer, or head and neck cancer.

### Administration and Dosage

Compounds (e.g., radioimmunoconjugates) and pharmaceutical compositions thereof disclosed herein may be administered by any of a variety of routes of administration, including systemic and local routes of administration

Systemic routes of administration include parenteral routes and enteral routes. In some embodiments, compounds (e.g., radioimmunoconjugates) or pharmaceutical compositions thereof are administered by a parenteral route, for example, intravenously, intraarterially, intraperitoneally, subcutaneously, intracranially, or intradermally. In some embodiments, compounds (e.g., radioimmunoconjugates) or pharmaceutical compositions thereof are administered intravenously. In some embodiments, compounds (e.g., radioimmunoconjugates) or pharmaceutical compositions thereof are administered by an enteral route of administration, for example, trans-gastrointestinal, or orally.

Local routes of administration include, but are not limited to, peritumoral injections and intratumoral injections.

Pharmaceutical compositions can be administered for radiation treatment planning, diagnostic, and/or therapeutic treatments. When administered for radiation treatment planning or diagnostic purposes, the compound (e.g., radioimmunoconjugate) may be administered to a subject in a diagnostically effective dose and/or an amount effective to determine the therapeutically effective dose. In therapeutic applications, pharmaceutical compositions may be administered to a subject (e.g., a human) already suffering from a condition (e.g., cancer) in an amount sufficient to cure or at least partially arrest the symptoms of the disorder and its complications. For example, in the treatment of cancer, an agent or compound that decreases, prevents, delays, suppresses, or arrests any symptom of the disease or condition would be therapeutically effective. A therapeutically effective amount of an agent or compound is not required to cure a disease or condition but may, for example, provide a treatment for a disease or condition such that the onset of the disease or condition is delayed, hindered, or prevented, such that the disease or condition symptoms are ameliorated, or such that the term of the disease or condition is changed. For example, the disease or condition may become less severe and/or recovery is accelerated in an individual. In some embodiments, a subject is administered a first dose of a compound (e.g., radioimmunoconjugate) or composition in an amount effective for radiation treatment planning, then administered a second dose or set of doses of the compound (e.g., radioimmunoconjugate) or composition in a therapeutically effective amount.

For treating cancer comprising cells expressing STEAP2, the method of this disclosure typically comprises administering to a subject (e.g., a human) in need thereof a first dose of a compound or composition provided above in an amount effective for radiation treatment planning, followed by administering subsequent doses of a compound or composition provided above in a therapeutically effective amount.

In some embodiments, the compound or composition administered in the first dose and the compound or composition administered in the second dose are the same.

In some embodiments, the compound or composition administered in the first dose and the compound or composition administered in the second dose are different.

Therapeutically effective amounts may depend on the severity of the disease or condition and other characteristics of the subject (e.g., weight). Therapeutically effective amounts of disclosed compounds (e.g., radioimmunoconjugates) and compositions for subjects (e.g., mammals such as humans) can be determined by the ordinarily skilled artisan with consideration of individual differences (e.g., differences in age, weight and the condition of the subject).

In some embodiments, disclosed compounds (e.g., radioimmunoconjugates) exhibit an enhanced ability to target cancer cells. In some embodiments, the effective amount of disclosed compounds (e.g., radioimmunoconjugates) is lower than (e.g., less than or equal to about 90%, 75%, 50%, 40%, 30%, 20%1, 5%1, 2%1, 0%, 8%, 7%, 6%, 5%, 4%, 3%, 2%, 1%, 0.5%, or 0.1% of) the equivalent dose for a therapeutic effect of the unconjugated, and/or non-radiolabeled targeting moiety. In some embodiments, the effective amount of disclosed compounds (e.g., radioimmunoconjugates) is about 90% lower than the equivalent dose for a therapeutic effect of the unconjugated, and/or non-radiolabeled targeting moiety. In some embodiments, the effective amount of disclosed compounds (e.g., radioimmunoconjugates) is about 75% lower than the equivalent dose for a therapeutic effect of the unconjugated, and/or non-radiolabeled targeting moiety. In some embodiments, the effective amount of disclosed compounds (e.g., radioimmunoconjugates) is about 50% lower than the equivalent dose for a therapeutic effect of the unconjugated, and/or non-radiolabeled targeting moiety. In some embodiments, the effective amount of disclosed compounds (e.g., radioimmunoconjugates) is about 40% lower than the equivalent dose for a therapeutic effect of the unconjugated, and/or non-radiolabeled targeting moiety. In some embodiments, the effective amount of disclosed compounds (e.g., radioimmunoconjugates) is about 30% lower than the equivalent dose for a therapeutic effect of the unconjugated, and/or non-radiolabeled targeting moiety. In some embodiments, the effective amount of disclosed compounds (e.g., radioimmunoconjugates) is about 20% lower than the equivalent dose for a therapeutic effect of the unconjugated, and/or non-radiolabeled targeting moiety. In some embodiments, the effective amount of disclosed compounds (e.g., radioimmunoconjugates) is about 15% lower than the equivalent dose for a therapeutic effect of the unconjugated, and/or non-radiolabeled targeting moiety. In some embodiments, the effective amount of disclosed compounds (e.g., radioimmunoconjugates) is about 12% lower than the equivalent dose for a therapeutic effect of the unconjugated, and/or non-radiolabeled targeting moiety. In some embodiments, the effective amount of disclosed compounds (e.g., radioimmunoconjugates) is about 10% lower than the equivalent dose for a therapeutic effect of the unconjugated, and/or non-radiolabeled targeting moiety. In some embodiments, the effective amount of disclosed compounds (e.g., radioimmunoconjugates) is about 8% lower than the equivalent dose for a therapeutic effect of the unconjugated, and/or non-radiolabeled targeting moiety. In some embodiments, the effective amount of disclosed compounds (e.g., radioimmunoconjugates) is about 7% lower than the equivalent dose for a therapeutic effect of the unconjugated, and/or non-radiolabeled targeting moiety. In some embodiments, the effective amount of disclosed compounds (e.g., radioimmunoconjugates) is about 6% lower than the equivalent dose for a therapeutic effect of the unconjugated, and/or non-radiolabeled targeting moiety. In some embodiments, the effective amount of disclosed compounds (e.g., radioimmunoconjugates) is about 5% lower than the equivalent dose for a therapeutic effect of the unconjugated, and/or non-radiolabeled targeting moiety. In some embodiments, the effective amount of disclosed compounds (e.g., radioimmunoconjugates) is about 4% lower than the equivalent dose for a therapeutic effect of the unconjugated, and/or non-radiolabeled targeting moiety. In some embodiments, the effective amount of disclosed compounds (e.g., radioimmunoconjugates) is about 3% lower than the equivalent dose for a therapeutic effect of the unconjugated, and/or non-radiolabeled targeting moiety. In some embodiments, the effective amount of disclosed compounds (e.g., radioimmunoconjugates) is about 2% lower than the equivalent dose for a therapeutic effect of the unconjugated, and/or non-radiolabeled targeting moiety. In some embodiments, the effective amount of disclosed compounds (e.g., radioimmunoconjugates) is about 1% lower than the equivalent dose for a therapeutic effect of the unconjugated, and/or non-radiolabeled targeting moiety. In some embodiments, the effective amount of disclosed compounds (e.g., radioimmunoconjugates) is about ≤1% lower than the equivalent dose for a therapeutic effect of the unconjugated, and/or non-radiolabeled targeting moiety.

Single or multiple administrations of pharmaceutical compositions disclosed herein including an effective amount can be carried out with dose levels and pattern being selected by the treating physician. Dose and administration schedule can be determined and adjusted based on the severity of the disease or condition in the subject, which may be monitored throughout the course of treatment according to the methods commonly practiced by clinicians or those described herein.

The following specific examples are to be construed as merely illustrative, and not limitative of the remainder of the disclosure in any way whatsoever.

### EXAMPLES

### Example 1. STEAP2 is Over-Expressed in Prostate Cancer

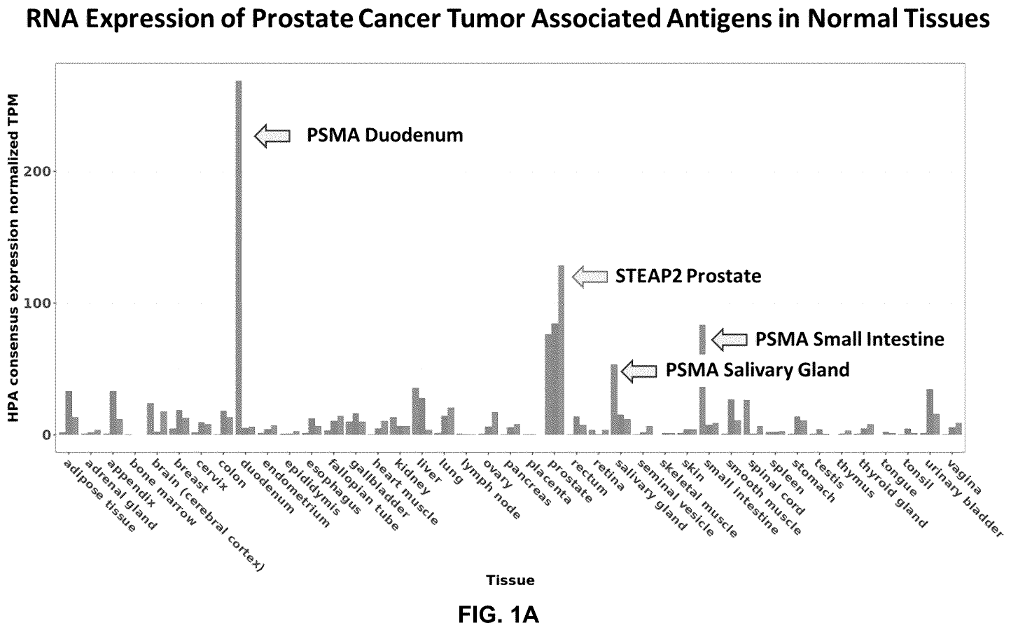

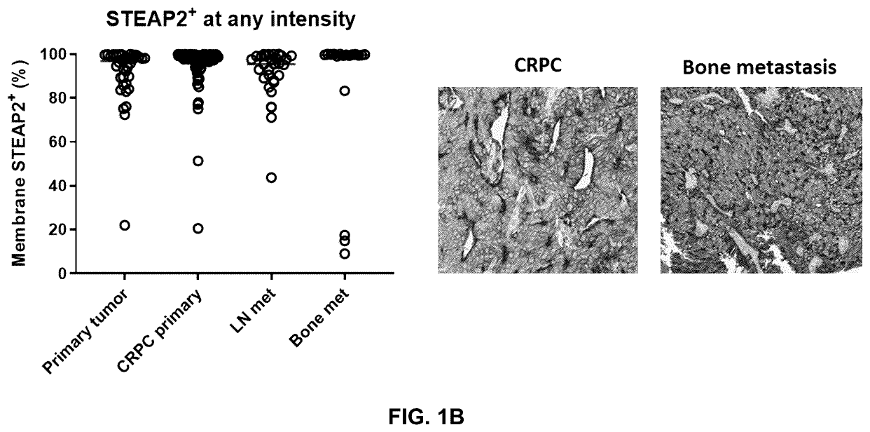

STEAP2 is a metalloreductase that reduces iron and copper to facilitate cellular uptake, metabolism, and proliferation, which is predominantly expressed in prostate cancer, with little expression in healthy tissue outside the prostate (FIG. 1A). A query with human protein atlas confirms that shows less RNA expression in vital organs than PSMA & STEAP1 (FIG. 1A). STEAP2 has high, homogeneous cell surface expression across all disease stages of prostate cancer, including metastases and castration-resistant prostate cancer (CRPC) (FIG. 1B).

The expression profile of STEAP2 was assessed using a validated IHC protocol to demonstrate STEAP2 expression in human tissues and human tumor tissues. Immunohistochemistry was carried out from a number of sections of tumors taken acquired from human subjects with primary (n=36), CRPC (n=78), lymph node metastases (n=30), or bone metastases (n=18). Expression across the collection of human tumors was similarly high.

### Example 2. Generation of Anti-STEAP2 Antibodies

40A3GL-LO14 is a human IgG1 κ antibody with reduced effector function (IgG1-TM) that binds to the extra-cellular domains (ecds) of STEAP2, a multi-pass membrane protein highly expressed on prostate cancer cells.

Parental mAb, 40A3, was isolated with a hybridoma campaign for which transgenic mice were immunized with STEAP2 expressing cells. It was not possible to drive expression and cell surface localization of STEAP2 in a non-prostate cell setting, such as Ad293 cells. Thus, a chimeric cell line was generated where the STEAP2 extracellular loops were grafted onto the backbone of the STEAP3 protein (STEAP3-2), to exploit the cell surface localization of STEAP3. Four- to six-week old transgenic female Del-1 mice (C57BL/6 background) were immunized with Ad293 cells overexpressing STEAP3-2. Three days post pre-fusion boost, splenocytes and lymph node cells were harvested. B-cells were isolated using a pan B-cell enrichment kit from Miltenyi. Isolated B-cells were further enriched by panning on irradiated STEAP2 knock out cell line. The antigen enriched B-cells were then fused to P3X63Ag8.653 (CRL-1580-ATCC) and seeded into 96 well plates in HAT selection medium. Supernatants from 96 well plates were screened using high through-put flow cytometry. Hybridomas specific to Ad293 OE STEAP2 were also tested for binding to primary cancer cell lines (LNCaP, LNCaP-STEAP2-KO) and additional STEAP family members. STEAP2-specific hybridomas were moved into limited dilution cloning. V-genes were rescued from all clones that retained specific binding to LNCaP. Recombinant antibodies were generated and used for further downstream testing.

Clone 40A3 was selected as a lead for further development based on cell-binding affinity, STEAP family member selectivity and human/murine cross-reactivity. The parental mAb was mutated by the introduction of germline leucine residue in framework three (FW3) of the VH domain and two deamidation motifs were removed from CDRs L1 and H3. The binding affinity of germ-lined variant, 40A3-LO7 (LO=lead clone number 7) was assessed on STEAP2 expressing LNCaP prostate cancer cells. 40A3-LO7 was shown to bind LNCaP cells with an on-cell binding affinity of 43.33 nM.

40A3-LO7 was affinity matured by site saturation mutagenesis and cell-based screening. Two affinity matured variants with limited background binding were subsequently identified, 40A3-LO11 (CDRL1_S30A CDRH2_V61P) and 40A3-LO14 (CDRL1_S30A CDRH2_V61P CDRH3_L97R). The combined CDRH2_V61P CDRH3_L97R substitution mutations in 40A3-LO14 improved binding by 26-fold relative to the starting antibody, 40A3-LO7.

The binding affinities for parental 40A3-LO7 and its affinity matured derivatives were assessed on LNCaP cells. 40A3-LO14 also showed the strongest binding affinity to LNCaP cells, with an EC50 of 1.67 nM. Variant 40A3-LO11 had a slightly poorer EC50 value of 2.38 nM. None of the variants tested showed binding to LNCaP STEAP2 CRISPR KO cells. Murine Cross Reactivity to AD293 muSTEAP3-2 was determined at 0.97 nM and 5.78 nM, respectively. Binding affinity, cross-reactivity and developability characteristics for each of LO11 and LO14 are summarized in Table 3 below:

TABLE 3

| Clone ID (IgG1- | Binding Affinity | Mu X-reactivity | Heat Stress (% | Photo stress (% |
|---|---|---|---|---|
| TM) | (LNCaP) | (AD293 STEAP3-2) | aggregate increase) | aggregate increase) |
| 40A3-LO14 | 1.67 nM | 0.97 nM | 0.27% | 1.75% |
| 40A3-LO11 | 2.38 nM | 5.78 nM | 0.24% | 0.8% |

### Example 3. General Materials and Methods for Radiopharmaceuticals

Lutetium-177 can be obtained from ITM Medical Isotopes as lutetium trichloride in a 0.05 N hydrochloric acid solution; indium-111, as indium trichloride in a 0.05 N hydrochloric acid solution, can be obtained from BWXT; and actinium-225 can be obtained as actinium-225 trinitrate from Oak Ridge National Laboratories or actinium-225 trichloride from Canadian Nuclear Laboratories.

Analytical HPLC-MS can be performed using a Waters Acquity HPLC-MS system comprised of a Waters Acquity Binary Solvent Manager, a Waters Acquity Sample Manager (samples cooled to 10° C.), a Water Acquity Column Manager (column temperature 30° C.), a Waters Acquity Photodiode Array Detector (monitoring at 254 nm and 214 nm), a Waters Acquity TQD with electrospray ionization and a Waters Acquity BEH C18, 2.1×50 (1.7 μm) column. Preparative HPLC can be performed using a Waters HPLC system comprised of a Waters 1525 Binary HPLC pump, a Waters 2489 UV/Visible Detector (monitoring at 254 nm and 214 nm) and a Waters XBridge Prep phenyl or C18 19×100 mm (5 μm) column.

HPLC elution method 1: Waters Acquity BEH C18 2.1×50 mm (1.7 μm) column; mobile phase A: H₂O (0.1% v/v TFA); mobile phase B: acetonitrile (0.1% v/v TFA); flow rate=0.3 mL/min; initial=90% A, 3-3.5 min=0% A, 4 min=90% A, 5 min=90% A.

HPLC elution method 2: Waters XBridge Prep Phenyl 19×100 mm (5 μm) column; mobile phase A: H₂O (0.1% v/v TFA); mobile phase B: acetonitrile (0.1% v/v TFA); flow rate: 10 mL/min; initial=80% A, 13 min=0% A.

HPLC elution method 3: Waters Acquity BEH C18 2.1×50 mm (1.7 μm) column; mobile phase A: H₂O (0.1% v/v TFA); mobile phase B: acetonitrile (0.1% v/v TFA); flow rate=0.3 mL/min; initial=90% A, 8 min=0% A, 10 min=0% A, 11 min=90% A, 12 min=90% A.

HPLC elution method 4: Waters XBridge Prep C18 OBD 19×100 mm (5 μm) column; mobile phase A: H₂O (0.1% v/v TFA); mobile phase B: acetonitrile (0.1% v/v TFA); flow rate: 10 mL/min; initial=80% A, 3 min=80% A, 13 min=20% A, 18 min=0% A.

HPLC elution method 5: Waters XBridge Prep C18 OBD 19×100 mm (5 μm) column; mobile phase A: H₂O (0.1% v/v TFA); mobile phase B: acetonitrile (0.1% v/v TFA); flow rate: 10 mL/min; initial=90% A, 3 min=90% A, 13 min=0% A, 20 min=0% A.

HPLC elution method 6: Waters XBridge Prep C18 OBD 19×100 mm (5 μm) column; mobile phase A: H₂O (0.1% v/v TFA); mobile phase B: acetonitrile (0.1% v/v TFA); flow rate: 10 mL/min; initial=75% A, 13 min=0% A, 15 min=0% A.

HPLC elution method 7: Waters XBridge Prep C18 OBD 19×100 mm (5 μm) column; mobile phase A: H₂O (0.1% v/v TFA); mobile phase B: acetonitrile (0.1% v/v TFA); flow rate: 10 mL/min; initial=80% A, 12 min=0% A, 15 min=0% A.

HPLC elution method 8: Waters XBridge Prep C18 OBD 19×100 mm (5 μm) column; mobile phase A: H₂O (0.1% v/v TFA); mobile phase B: acetonitrile (0.1% v/v TFA); flow rate: 10 mL/min; initial=90% A, 12 min=0% A, 15 min=0% A.

Analytical Size Exclusion Chromatography (SEC) can be performed using a Waters system comprised of a Waters 1525 Binary HPLC pump, a Waters 2489 UV/Visible Detector (monitoring at 280 nm), a Bioscan Flow Count radiodetector (FC-3300) and TOSOH TSKgel G3000SWxl, 7.8×300 mm column. The isocratic SEC method can have a flow rate of, e.g., mL/min, with a mobile phase of 0.1 M phosphate, 0.6 M NaCl, 0.025% sodium azide, pH=7.

MALDI-MS (positive ion) can be performed using a MALDI Bruker Ultraflextreme Spectrometer.

Radio thin-layer chromatography (radioTLC) can be performed with Bioscan AR-2000 Imaging Scanner, and can be carried out on iTLC-SG glass microfiber chromatography paper (Agilent Technologies, SGI0001) plates using citrate buffer (0.1 M, pH 5.5).

### Example 4. Synthesis of 4-{[11-Oxo-11-(2,3,5,6-Tetrafluorophenoxy)Undecyl]Carbamoyl}-2-[4,7,10-tris(carboxymethyl)-1,4,7,10-tetraazacyclododecan-1-yl]butanoic acid (Compound B)

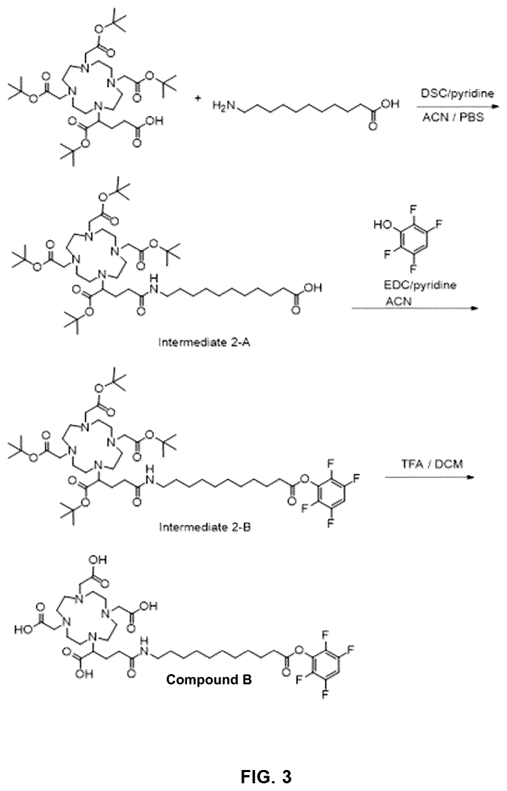

A bifunctional chelate, 4-{[11-oxo-11-(2,3,5,6-tetrafluorophenoxy)undecyl]carbamoyl}-2-[4,7,10-tris(carboxymethyl)-1,4,7,10-tetraazacyclododecan-1-yl]butanoic acid (Compound B), can be synthesized according to the scheme provided in FIG. 3. To a solution of 5-(tert-butoxy)-5-oxo-4-(4,7,10-tris(2-(tert-butoxy)-2-oxoethyl)-1,4,7,10-tetraazacyclododecan-1-yl)pentanoic acid (DOTA-GA-(tBu)₄, 50 mg, 0.07 mmol) in ACN (2.0 mL), DSC (50 mg, 0.21 mmol) is added, followed by pyridine, (0.20 mL, 2.48 mmol). The reaction is stirred at room temperature for 1 hour. To the reaction mixture is added 11-aminoundecanoic acid, (70 mg, 0.36 mmol) followed by PBS solution (1.0 mL) at room temperature. The reaction is stirred for 72 hours at room temperature. The reaction mixture is filtered with a syringe filter and purified directly by Prep-HPLC using method 6 to yield Intermediate 2-A.

To a solution of Intermediate 2-A (40 mg, 0.03 mmol), TFP (90 mg, 0.54 mmol) and EDC (40 mg, 0.27 mmol) in ACN (1.0 mL) is added pyridine (0.05 mL, 50 mg, 0.62 mmol) at room temperature. The solution is stirred at room temperature for 24 hours. The reaction is purified directly by Prep-HPLC using method 7 to provide Intermediate 2-B as a wax after concentration using a Biotage V10 Rapid Evaporator.

Intermediate 2-B is dissolved in DCM/TFA (1.0 mL/2.0 mL) and allowed to stir at room temperature for 24 hours. The reaction is concentrated by air stream and purified directly by Prep-HPLC using method 8 to yield Compound B as a clear wax after concentration. An aliquot is analyzed by HPLC-MS elution method 3.

¹H NMR (600 MHz, DMSO-d₆) δ 7.99-7.88 (m, 1H), 7.82 (t, J=5.5 Hz, 1H), 3.78 (broad s, 4H), 3.43 (broad s, 12H), 3.08 (broad s, 4H), 3.00 (m, 3H), 2.93 (broad s, 3H), 2.77 (t, J=7.2 Hz, 2H), 2.30 (broad s, 2H), 1.88 (broad s, 2H), 1.66 (p, J=7.3 Hz, 2H), 1.36 (m, 4H), 1.32-1.20 (m, 9H).

### Example 5. Synthesis of 4-{[2-(2-{2-[3-Oxo-3-(2,3,5,6-Tetrafluorophenoxy)Propoxy]ethoxy}ethoxy)ethyl]carbamoyl}-2-[4,7,10-tris(carboxymethyl)-1,4,7,10-tetraazacyclododecan-1-yl]butanoic acid (Compound C)

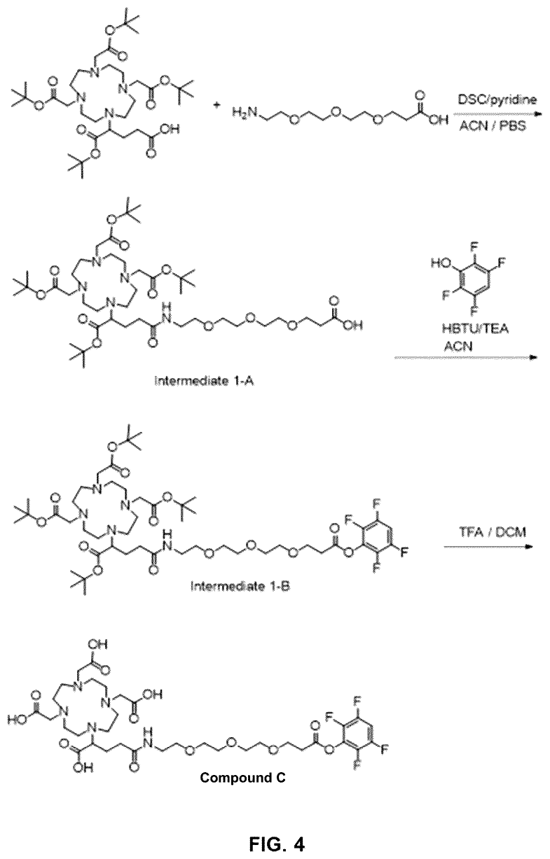

A bifunctional chelate, 4-{[2-(2-{2-[3-oxo-3-(2,3,5,6-tetrafluorophenoxy)propoxy]ethoxy}ethoxy)ethyl]carbamoyl}-2-[4,7,10-tris(carboxymethyl)-1,4,7,10-tetraazacyclododecan-1-yl]butanoic acid (Compound C), is synthesized according to the scheme provided in FIG. 4.

To a solution of 5-(tert-butoxy)-5-oxo-4-(4,7,10-tris(2-(tert-butoxy)-2-oxoethyl)-1,4,7,10-tetraazacyclododecan-1-yl)pentanoic acid (DOTA-GA(tBu)₄, 100 mg, 0.143 mmol) in ACN (8.0 mL) is added DSC (73 mg, 0.285 mmol) and pyridine (0.80 mL, 9.89 mmol). The reaction mixture is stirred for 90 min at ambient temperature. This solution is added to a semi-solution of amino-PEG3-acid (63 mg, 0.285 mmol in 1.2 mL of DMF) in a 100 mL round bottom flask. After 4 hours at ambient temperature, the reaction is worked up by concentrating to dryness under a stream of air. The crude material is purified by HPLC elution method 2 (dissolved the crude in 6 mL of 20% ACN/H₂O). The fractions containing product are pooled and concentrated under vacuum and then co-evaporated with ACN (3×2 mL).

To a vial containing Intermediate 1-A (82 mg, 60 μmol) is added ACN (2 mL), NEt₃ (50 μL, 360 μmol, 6 equiv.), HBTU (23 mg, 60 μmol, 1 equiv) and a TFP solution (50 mg, 300 mol, 5 equiv., dissolved in 250 μL of ACN). The resulting clear solution is stirred at ambient temperature for 3 hours. The reaction is worked up by concentrating the solution to dryness under an air stream and is then diluted with ACN/H₂O (1:1, 3 mL total) and purified on preparative HPLC using elution method 4. Fractions containing product are pooled and concentrated under vacuum and then co-evaporated with ACN (3×2 mL). Intermediate 1-B is obtained as a clear residue.

To a vial containing Intermediate 1-B (67 mg, 64 μmol) is added DCM (2 mL) and TFA (2 mL). The resulting solution is stirred at ambient temperature for 16 hours. Additional, TFA (2 mL) is added, and the reaction is stirred at ambient temperature for 6 hours. The reaction is concentrated to dryness under an air stream, with the crude product being finally dissolved in ACN/H₂O (1 mL of 10% ACN/H₂O). The crude reaction solution is then purified by preparative HPLC using elution method 5. The fractions containing product are pooled, frozen and lyophilized. Compound C is obtained as a white solid. An aliquot is analyzed by HPLC-MS elution method 3.

¹H NMR (DMSO-d₆, 600 MHz) δ 7.97-7.91 (m, 2H), 3.77 (t, 2H, J=6.0 Hz), 3.58-3.55 (m, 2H), 3.53-3.48 (m, 8H), 3.44-3.38 (m, 10H), 3.23-3.08 (m, 11H), 3.02 (t, 2H, J=6.0 Hz), 2.93 (broad s, 4H), 2.30 (broad s, 2H), 1.87 (broad s, 2H).

### Example 6. Conjugation and Radiolabeling for Synthesis of Radioimmunoconjugates Comprising STEAP2 Antibody

### Synthesis of STEAP2 Immunoconjugate

The anti-STEAP2 mAb (40A3-LO14 hIgG-TM) is obtained in solution at a concentration of 50.0 mg/mL (60 mM histidine, 240 mM sucrose, pH 6.0). The mAb is reformulated into acetate buffer (100 mM, pH 6.5) using HiTrap Desalting columns (Cytiva). The mAb is diluted to a concentration of ˜15 mg/mL with acetate buffer and the pH adjusted to between 9 and 10 with carbonate buffer. A solution of 4-{[2-(2-{2-[3-oxo-3-(2,3,5,6-tetrafluorophenoxy)propoxy]ethoxy}ethoxy)ethyl]carbamoyl}-2-[4,7,10-tris(carboxymethyl)-1,4,7,10-tetraazacyclododecan-1-yl]butanoic acid (5 mg/mL in 0.001 M HCl, 6 eq) is added to the mAb solution and the reaction allowed to stand at room temperature for 1 hour. The resulting immunoconjugate can be purified and concentrated by hydrophobic interaction chromatography and reformulated into acetate buffer (pH 6.5) using HiTrap Desalting columns. Identities of immunoconjugates can be confirmed by, e.g., MALDI-TOF, where typical chelate to antibody ratios (CARs) of 5-5.5 are obtained.

### Radiosynthesis of Radioimmunoconjugate [¹¹¹In]-STEAP2

To a 1.5 mL Eppendorf tube is added the STEAP2 immunoconjugate (20.6 μL, 7.29 mg/mL), acetate buffer (100 mM, pH 6.5, 75.2 μL) and a 0.05±0.01 M HCl solution of [¹¹¹In]InCl₃ (4.2 μL, 1.85 mCi). After 60 minutes at ambient temperature, radioTLC analysis of the reaction mixture (iTLC-SG plates, 5% methanol in 0.02 M citrate buffer as the mobile phase) indicated a radiochemical conversion (RCC) of 95%. Purification was carried out using a 1 mL column packed with Sephadex G50 resin (hydrated with acetate buffer). The product fractions were eluted using acetate buffer and combined. Acetate buffer solutions of sodium L-ascorbate and diethylenetriamine-pentaacetic acid calcium trisodium salt hydrate (DTPA) were added to give a final formulation of 10 mM ascorbate and 1 mM DTPA. Analysis of the resulting formulation by radioTLC and SEC-HPLC at end-of-synthesis (EOS) indicated the formation of [¹¹¹In]-STEAP2 (374 μL, 0.287 mg/mL, 10.5 mCi/mg, 99% radiochemical purity and >95% chemical purity).

### Radiosynthesis of Radioimmunoconjugate [¹⁷⁷Lu]-STEAP2

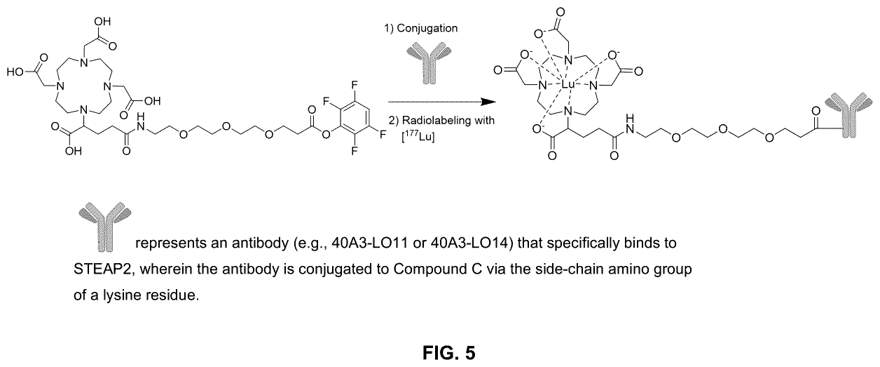

To a 1.5 mL Eppendorf tube is added the STEAP2 immunoconjugate (20.6 μL, 7.29 mg/mL), acetate buffer (100 mM, pH 6.5, 70.8 μL) and a 0.01 M HCl solution of [¹⁷⁷Lu]LuCl₃ (8.6 μL, 1.93 mCi). After 60 minutes at ambient temperature, radioTLC analysis of the reaction mixture (iTLC-SG plates, 5% methanol in 0.02 M citrate buffer as the mobile phase) indicated a radiochemical conversion (RCC) of 94%. Purification was carried out using a 1 mL column packed with Sephadex G50 resin (hydrated with acetate buffer). The product fractions were eluted using acetate buffer and combined. Acetate buffer solutions of sodium L-ascorbate and diethylenetriamine-pentaacetic acid calcium trisodium salt hydrate (DTPA) were added to give a final formulation of 10 mM ascorbate and 1 mM DTPA. Analysis of the resulting formulation by radioTLC and SEC-HPLC at end-of-synthesis (EOS) indicated the formation of [¹⁷⁷Lu]-STEAP2 (374 μL, 0.250 mg/mL, 13.1 mCi/mg, 98% radiochemical purity and >95% chemical purity). The structure of [¹⁷⁷Lu]-STEAP2 is shown in FIG. 2C and its synthetic scheme is depicted in FIG. 5.

### Radiosynthesis of Radioimmunoconjugate [²²⁵Ac]-STEAP2

To a 1.5 mL Eppendorf tube is added the STEAP2 immunoconjugate (95.1 μL, 5.89 mg/mL), acetate buffer (100 mM, pH 6.5, 70.8 μL) and a 0.001 M HCl solution of [²²⁵Ac]AcCl₃ (31.5 μL, 32.8 μCi). After 120 minutes at 37° C., radioTLC analysis of the reaction mixture (iTLC-SG plates, 5% methanol in 0.02 M citrate buffer as the mobile phase) indicated a radiochemical conversion (RCC) of 99%. Purification was carried out using a 1 mL column packed with Sephadex G50 resin (hydrated with acetate buffer). The product fractions were eluted using acetate buffer and combined. Acetate buffer solutions of sodium L-ascorbate and diethylenetriamine-pentaacetic acid calcium trisodium salt hydrate (DTPA) were added to give a final formulation of 10 mM ascorbate and 1 mM DTPA. Analysis of the resulting formulation by radioTLC and SEC-HPLC at end-of-synthesis (EOS) indicated the formation of [²²⁵Ac]-STEAP2 (463 μL, 0.845 mg/mL, 0.066 mCi/mg, >99% radiochemical purity and >95% chemical purity). The structure of [²²⁵Ac]-STEAP2 is shown in FIG. 2D.

Synthesis of hIgG Isotype Immunoconjugate

The hIgG mAb was obtained in solution at a concentration of 77.43 mg/mL (25 mM histidine, 150 mM sucrose, pH 6.0). The mAb was reformulated into PBS using a 1 mL column packed with Sephadex G50 resin (hydrated with PBS). This was diluted to a concentration of ˜7 mg/mL and the pH adjusted to between 9 and 10 using carbonate buffer. A solution of 4-{[2-(2-{2-[3-oxo-3-(2,3,5,6-tetrafluorophenoxy)propoxy]ethoxy}ethoxy)ethyl]carbamoyl}-2-[4,7,10-tris(carboxymethyl)-1,4,7,10-tetraazacyclododecan-1-yl]butanoic acid (5 mg/mL in 0.001 M HCl, 6 eq) is added to the mAb solution and the reaction allowed to stand at room temperature for 1 hour. The antibody is then purified and reconstituted into acetate buffer (100 mM, pH 6.5) using 0.5 mL Amicon centrifugal filters (50 kDa cutoff). Identities of immunoconjugates can be confirmed by, e.g., MALDI-TOF, where typical chelate to antibody ratios (CARs) of 3-4 are obtained.

### Radiosynthesis of Radioimmunoconjugate [²²⁵Ac]-hIgG Isotype

To a 1.5 mL Eppendorf tube is added the hIgG isotype immunoconjugate (55.3 μL, 5.43 mg/mL), acetate buffer (100 mM, pH 6.5, 127.8 μL) and a 0.001 M HCl solution of [²²⁵Ac]AcCl₃ (17.5 μL, 17.9 μCi). After 120 minutes at 37° C., radioTLC analysis of the reaction mixture (iTLC-SG plates, 5% methanol in 0.02 M citrate buffer as the mobile phase) indicated a radiochemical conversion (RCC) of 95%. Purification was carried out using a 1 mL column packed with Sephadex G50 resin (hydrated with acetate buffer). The product fractions were eluted using acetate buffer and combined. Acetate buffer solutions of sodium L-ascorbate and diethylenetriamine-pentaacetic acid calcium trisodium salt hydrate (DTPA) were added to give a final formulation of 10 mM ascorbate and 1 mM DTPA. Analysis of the resulting formulation by radioTLC and SEC-HPLC at end-of-synthesis (EOS) indicated the formation of [²²⁵Ac]-hIgG (463 μL, 0.441 mg/mL, 0.0675 mCi/mg, 98% radiochemical purity and >95% chemical purity).

### Example 7. In Vitro Binding of [¹⁷⁷Lu]-Compound C-Anti-STEAP2 Conjugate

A study was conducted to evaluate the receptor binding affinity of [¹⁷⁷Lu]-Compound C-anti-STEAP2 conjugate, i.e., [¹⁷⁷Lu]-STEAP2 (40A3-LO11) and [¹⁷⁷Lu]-STEAP2 (40A3-LO14), with three different cell lines: C4-2 cells, 22RV1 cells, and LNCaP cells. This study followed the procedures described below.

The purpose of this assay was to ensure that the radioimmunoconjugates maintained the binding characteristics of the native antibody in STEAP2 expressing cell lines including C4-2, 22RV1 and LNCaP. One day prior to the experiment, cells (1.5-2×10⁵) were seeded in 48-well microplates in 500 μL supplemented medium. At the start of the assay, cells were washed with PBS once and then treated with either Binding Buffer or 5 μM cold antibody [total binding (TB) and non-specific binding (NSB) respectively]. Plates were incubated at 4° C. for about one hour with mild shaking. Following incubation, both SB and NSB cells were treated with increasing concentrations of [¹⁷⁷Lu]-STEAP2 (40A3-LO11) or [¹⁷⁷Lu]-STEAP2 (40A3-LO14) (0.098 nM to 50 nM) and were incubated at 4° C. for about two hours with mild shaking.

Following incubation, the cells were washed twice with PBS and were then lysed with 1% Triton-X-100. The lysates were transferred to gamma counting tubes and run along with the standard of [¹⁷⁷Lu]-STEAP2 (40A3-LO11) or [¹⁷⁷Lu]-STEAP2 (40A3-LO14) on the Wizard 1470 gamma counter to determine the amount of radioactivity in counts per minute (CPM) for each lysate. The remaining lysate from each well (25 μL) was used for analyzing the protein content using a standard protein quantification assay.

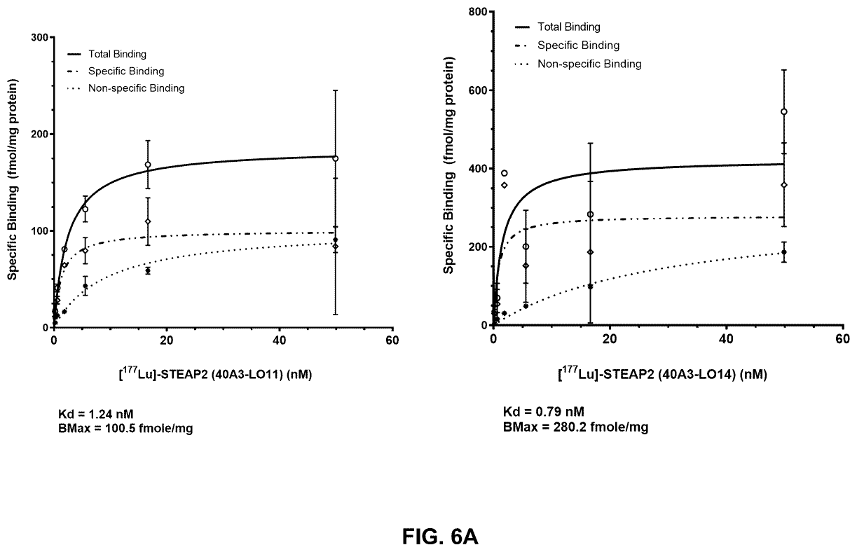

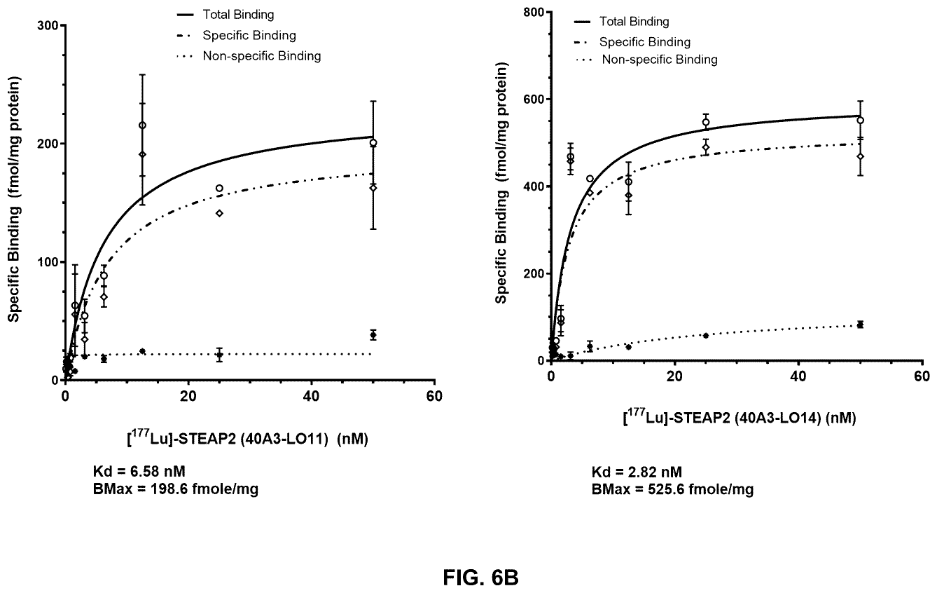

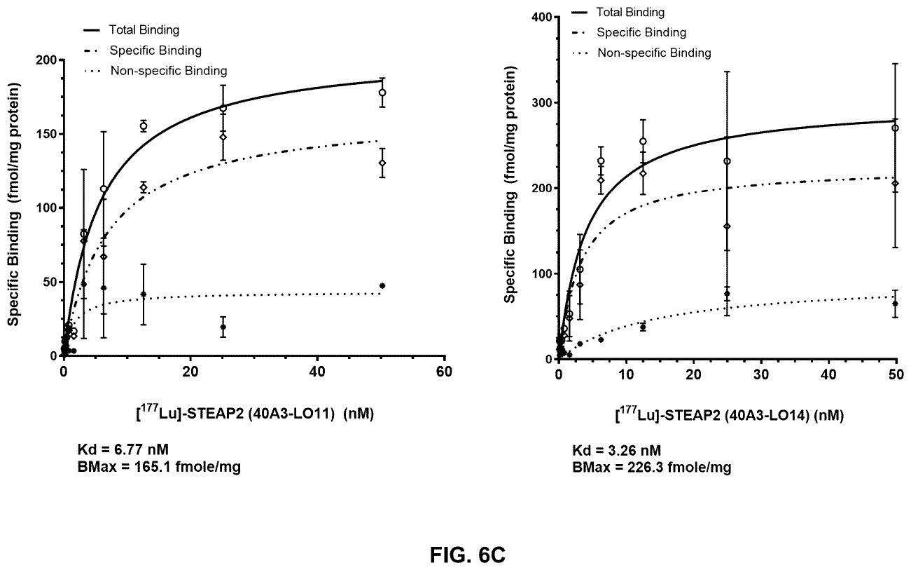

Total, specific, and non-specific binding values (fmol/mg) were plotted against conjugate concentration as shown in FIGS. 6A-6C. The Kd, and Bmax were derived by curve fitting of the specific binding data to a single-site hyperbola model (Graph Pad Prism Software, version 9). The binding data revealed desirable values for Kd. The highest Bmax (fmol/mg) for 40A3-LO11 was observed in C4-2 (199±36) and 22RV1 (165±18) followed by LNCaP (100±19). The highest Bmax (fmol/mg) for 40A3-LO14 was observed in C4-2 (525±52) and LNCaP (280±73) followed by 22RV1 (226±35).

It was observed that the STEAP2 conjugate, i.e., [¹⁷⁷Lu]-STEAP2 (40A3-LO14), exhibited higher binding affinities of about 3.2 nM with the 22RV1 cells, about 2.8 nM with the C4-2 cells, and about 0.79 nM with the LNCaP cells, compared to [¹⁷⁷Lu]-STEAP2 (40A3-LO11), which exhibited binding affinities of about 6.7 nM with the 22RV1 cells, about 6.9 nM with the C4-2 cells, and about 1.2 nM with the LNCaP cells.

### Example 8. Internalization Evaluation of [¹⁷⁷Lu]-Compound C-Anti-STEAP2 Conjugate

This internalization assay was designed to determine the degree of cell retention of radiolabeled—linker antibody derivatives. The assay relies on the inherent ability of the STEAP2 receptor to internalize when bound to antibody and the ability to track radiolabeled compounds. Here, a constant amount of radioimmunoconjugate is incubated with three different cell lines for a fixed duration of time and residualizations are determined by calculating the amount of internalized radioactivity as a percentage of the total cell-associated activity.

A study was conducted to evaluate the internalization of a [¹⁷⁷Lu]-Compound C-anti-STEAP2 conjugate, i.e., [¹⁷⁷Lu]-STEAP2 (40A3-LO11) and [¹⁷⁷Lu]-STEAP2 (40A3-LO14), with three different cell lines of C4-2 cells, 22RV1 cells, and LNCaP cells, following the protocol described below.

This assay was designed to determine the degree of cell retention of the radioimmunoconjugate [¹⁷⁷Lu]-STEAP2 (40A3-LO11) and [¹⁷⁷Lu]-STEAP2 (40A3-LO14). Briefly, the above-mentioned cell lines were seeded in three 24-well plates at a concentration of 2.5×10⁵ cells/well in complete medium (for 0 h, 2 h and 24 h incubation time). Next day, media was decanted, the cells were washed once with sterile PBS and then treated with either [¹⁷⁷Lu]-STEAP2 (40A3-LO11) or [¹⁷⁷Lu]-STEAP2 (40A3-LO14) (4 nM) for 2 hours at 37° C. After incubation, all the plates were immediately placed on ice and medium was discarded into pre-labeled (non-bound) gamma counting tubes. Cells were washed once with sterile PBS, gently shaken, and decanted into the (non-bound labeled) gamma tubes. Strong acid wash buffer (pH 2.5, 500 μL) was added into 0-hour time point for 5 minutes at 4° C., and then buffer was collected into pre-labeled (membrane-bound) gamma counting tubes. Cells were then lysed with 300 μL 1% Triton X-100 for 30 minutes at room temperature with gentle shaking. 250 μL of the cell lysate was transferred into gamma counting tubes and counted for 10 minutes. Mild acid wash buffer (pH 4.6, 500 μL) was added to 2-hour and 24-hour plates for 15 min at 4° C. The buffer was then collected into pre-labeled (membrane-bound) gamma counting tubes. 1 mL of warmed media was added to the plates for further incubation at 37° C. for 2 and 24 hours respectively. Following the prescribed incubation times, plates were placed on ice and processed in the following manner-media was decanted and collected into pre-labeled (efflux) gamma tubes. Plates were then washed once with 1 mL cold PBS and added into efflux tubes. Strong acid wash buffer was added to all wells and plates were incubated for 5 minutes on ice. The acid wash fraction was then collected into pre-labeled (recycled) gamma tubes. Cells were lysed with 300 μL 1% Triton X—100 for 30 minutes at room temperature. 250 μL of the cell lysate was transferred into pre-labeled (retained) gamma counting tubes and counted for 10 minutes. 25 μL of the cell lysate fraction was transferred to a 96-well plate for protein quantification (Pierce BCA Protein Assay).

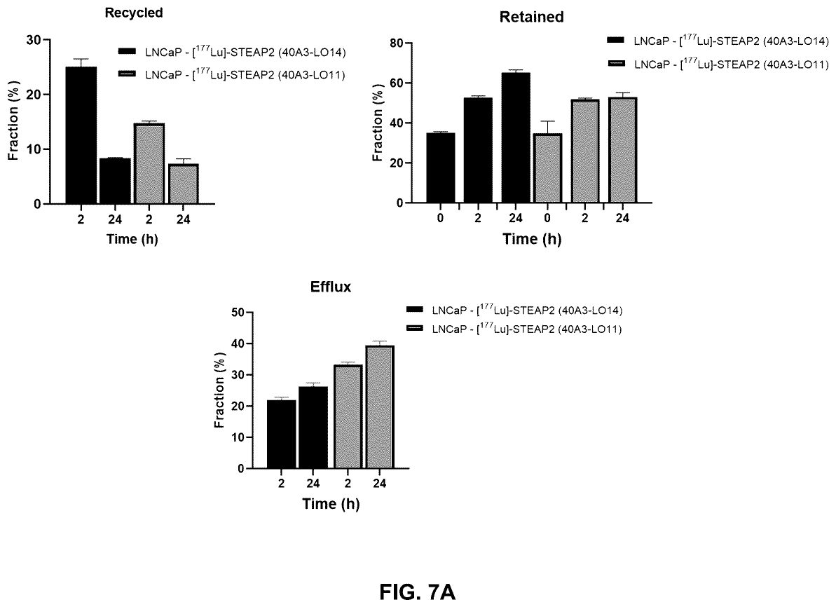

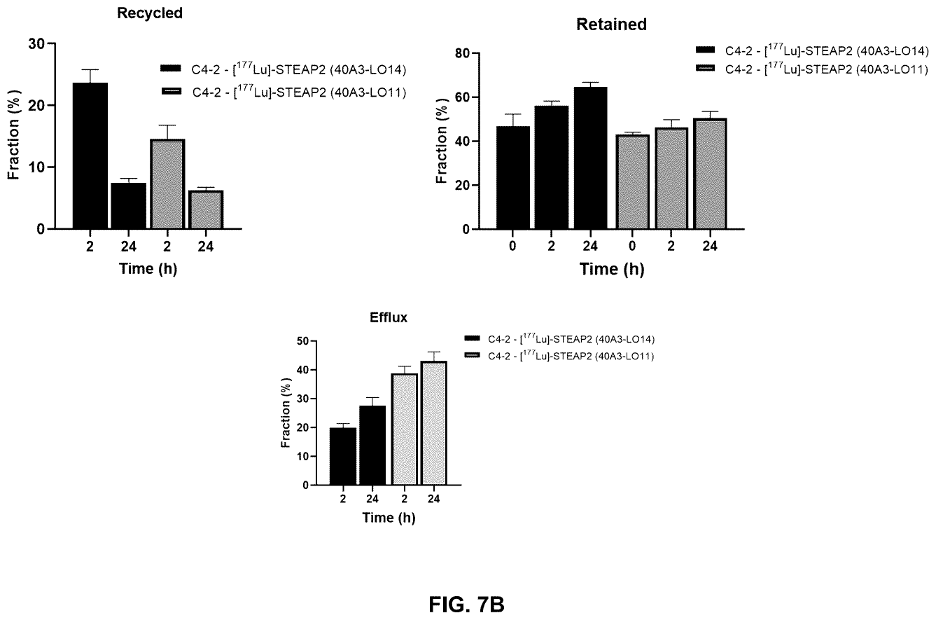

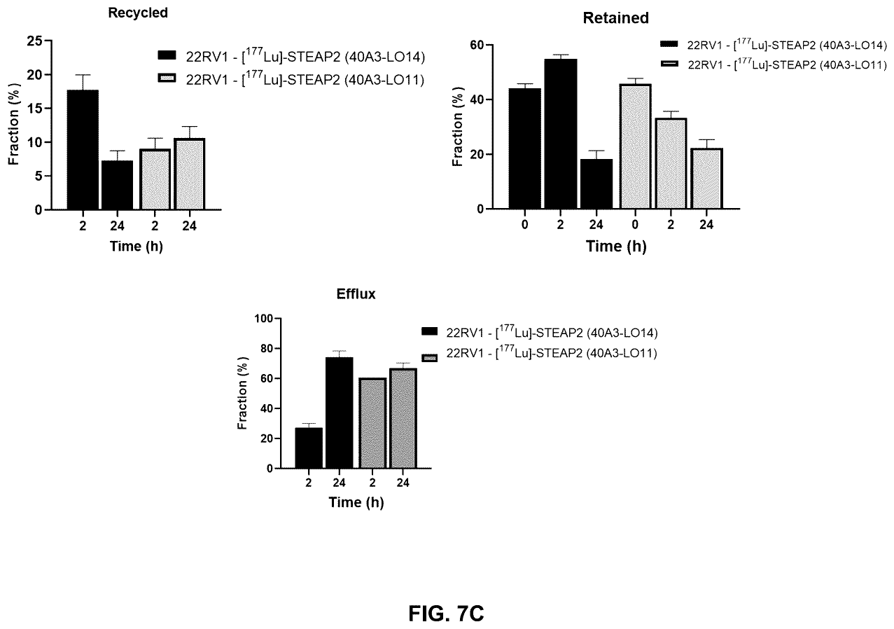

The results from the internalization study are shown in FIG. 7. Percent residualization was determined as CPM (lysate) or CPM (efflux+recycled+lysate). For [¹⁷⁷Lu]-STEAP2 (40A3-LO11), the lowest % efflux at 24 hours post-incubation was observed in LNCaP, followed by C4-2, and 22RV1 (about 39.55%, 43.1%, and 66.7% respectively). For [¹⁷⁷Lu]-STEAP2 (40A3-LO14), the lowest % efflux at 24 hours post-incubation was observed in LNCaP, followed by C4-2, and 22RV1 (about 26.3%, 27.69%, and 74% respectively). There seemed to be a correlation between the expression level STEAP2 receptors and cellular retention in this case for both 40A3-LO11 and 40A3-LO14.

### Example 9. In Vivo Biodistribution of [¹⁷⁷Lu]-Compound C-Anti-STEAP2 Conjugate in 22RV1 and C4-2 Animal Models

Two different cell line xenograft mouse models were used to assess the in vivo biodistribution of [¹⁷⁷Lu]-DOTA-anti-STEAP2 conjugate, i.e., [¹⁷⁷Lu]-STEAP2 (40A3-LO14), following the below protocol.

Tumor inoculations: Cells were washed with PBS and detached with 0.25% trypsin EDTA. The harvested cells were resuspended in a ready to use Cultrex concentrate at the following concentrations:
22RV1: 125×10⁶ cells/mL
C4-2: 125×10⁶ cells/mL

Four to 6-week-old male Athymic NCr-nu/nu mice (Charles River Laboratories) were injected subcutaneously into the right flank with 100 μL of the mixture. Radioactive injections started at about 15-25 days post inoculation when tumor volume reached 150-200 mm3.

Biodistribution studies: Six groups of 3 mice with subcutaneous tumors (described above) were injected intravenously via the lateral tail vein with 200 μL of [¹⁷⁷Lu]-STEAP2 (40A3-LO14) containing approximately 0.74 MBq of ¹⁷⁷Lu (about 2 μg of antibody). At specified timepoints (4 h, 24 h, 72 h, 96 h, 168 h, and 336H) post injection, one group per timepoint were anesthetized with isoflurane, exsanguinated via cardiac puncture then euthanized for blood and different organ collection by dissection. Tumor and organs were rinsed with PBS of any residual blood, blotted dry and collected into pre-weighed gamma counting tubes. Radiation counts per minute contained in tissue samples were measured using a gamma counter then converted to decay corrected μCi of activity using a calibration standard. Activity measurements and sample weights were used to calculate the percent of injected dose per gram of tissue weight (% ID/g).

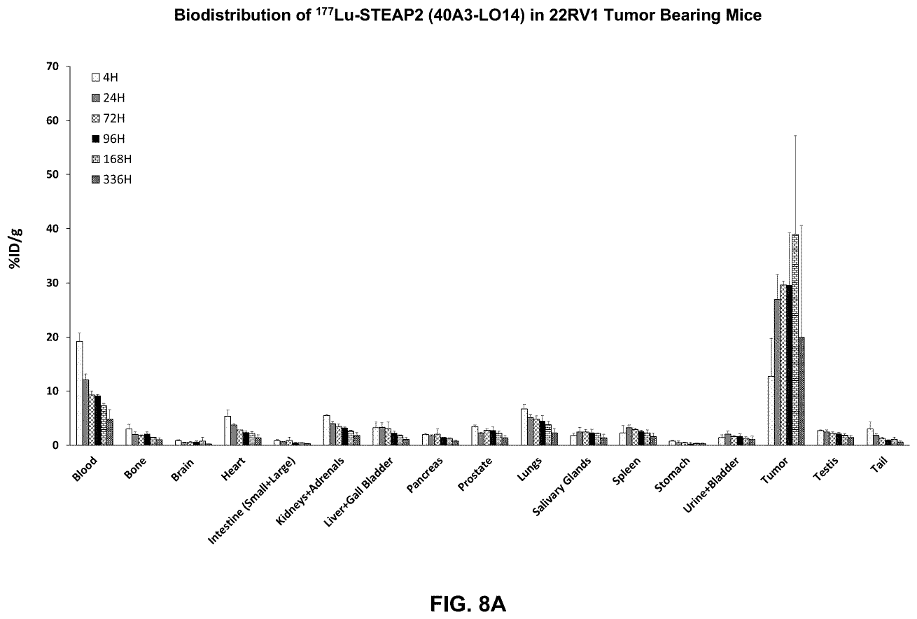

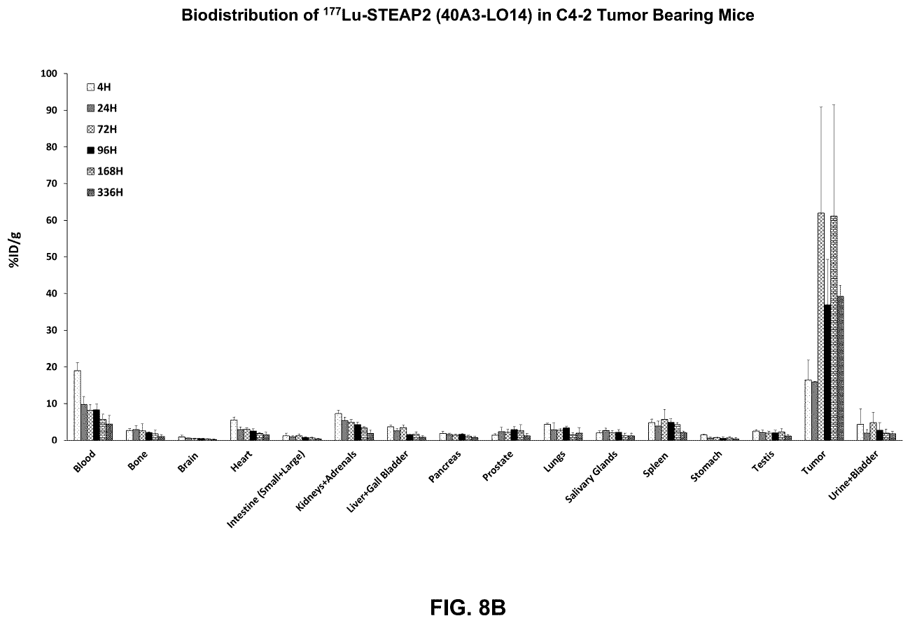

Results were expressed as the percentage injected dose per gram of tissue (% ID/g) and are depicted in FIGS. 8A-8B. Biodistribution study of [¹⁷⁷Lu]-STEAP2 (40A3-LO14) showed the typical biodistribution profile for IgGs with acceptable levels of uptake in normal organs. The highest tumor uptake (% ID/g) observed in C4-2 xenograft was greater than that in 22RV1 xenograft [about 61% (168 h), and about 38% (168 h) respectively].

### Example 10. In Vivo Efficacy of [²²⁵Ac]-Compound C-Anti-STEAP2 Conjugate in an Animal Model

A study was designed to evaluate the efficacy for different doses of actinium-225 labeled radioimmunoconjugate, i.e., [²²⁵Ac]-STEAP2 (40A3-LO14), as compared to the cold antibody alone, vehicle control, and isotype control.

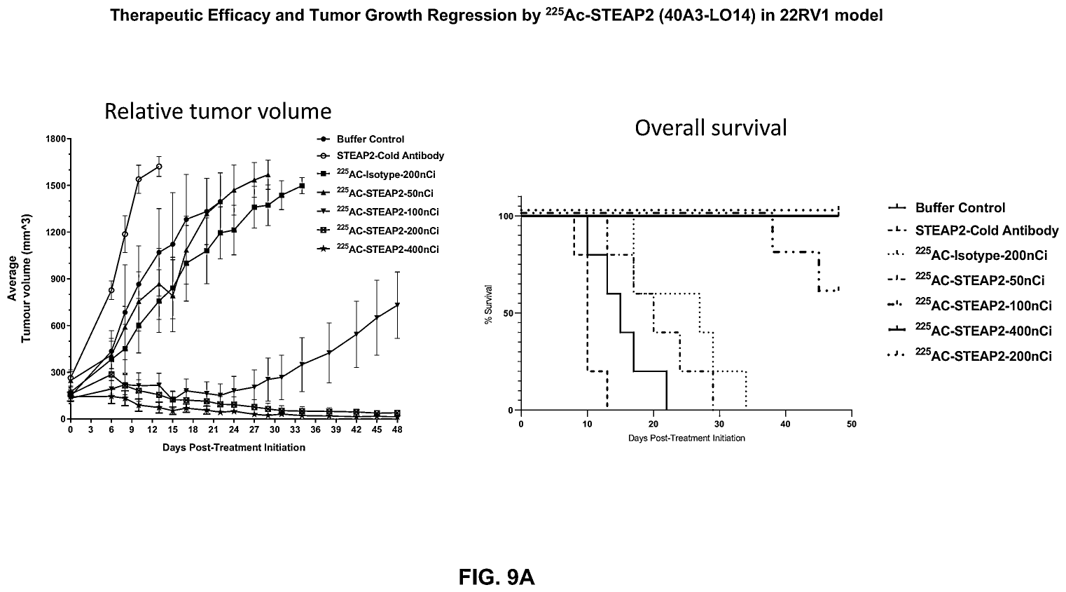

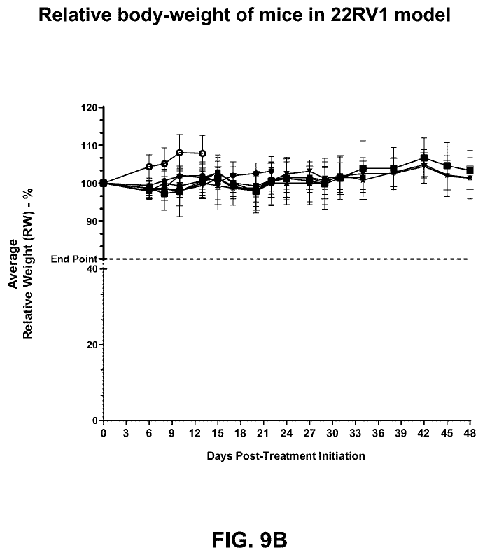

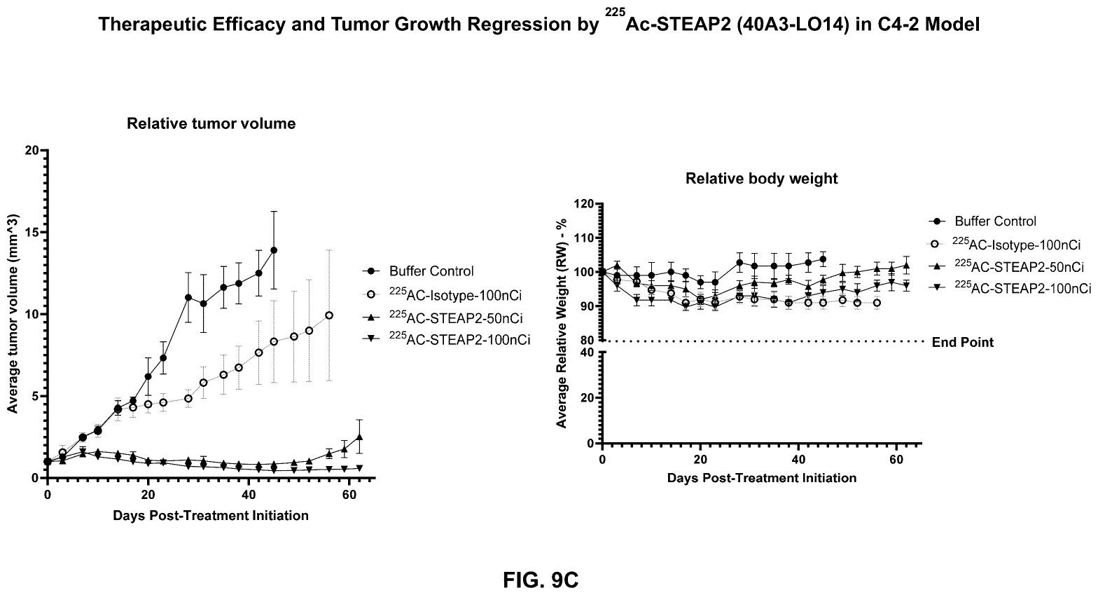

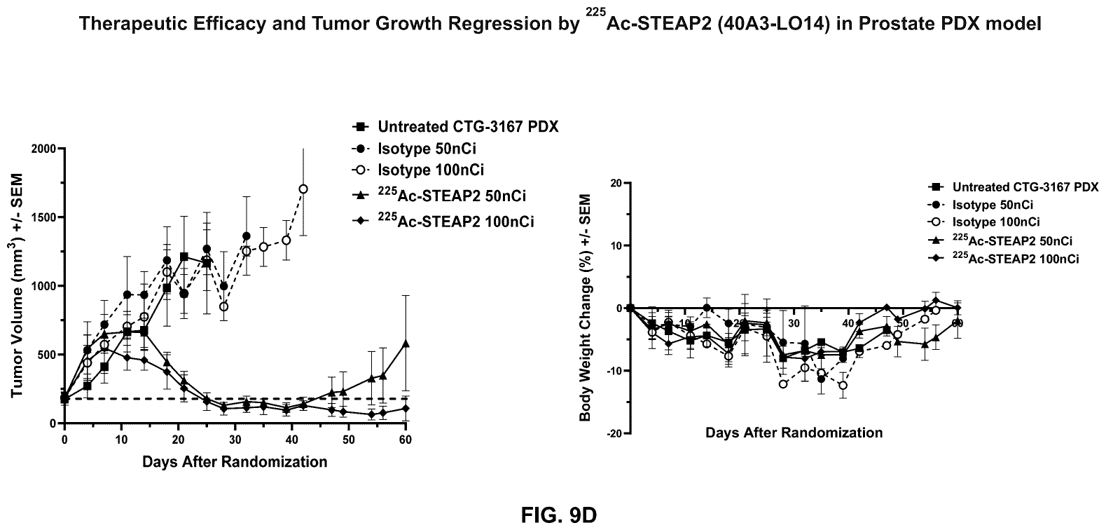

The Efficacy study was done with up to four increasing doses of [²²⁵Ac]-STEAP2 (40A3-LO14) and compared to the cold-antibody, vehicle control, and isotype control. Therapeutic efficacy studies were carried out using 22RV1 or C4-2 tumor xenografts. For the study, 4-7 groups of tumor-bearing animals (n=5 for 22RV1, n=4 for C4-2) were injected intravenously via the lateral tail vein with 200 μL of compounds. [²²⁵Ac]-STEAP2 (40A3-LO14) was dosed at 50-400 nanocuries (nCi) of activity formulated in 20 mM sodium citrate pH 5.5, 0.82% NaCl, and 0.01% Tween 80 buffer. As a control, non-radiolabeled, non-conjugated antibody was administered at a protein mass equivalent corresponding to the highest radioactivity dose of [²²⁵Ac]-STEAP2 (40A3-LO14) tested in a study. Tumor measurements were taken 2-3 times per week for at least 60 days with vernier calipers in two dimensions. Tumor length was defined as the longest dimension, width was measured perpendicular to the tumor length. At the same time animals were weighed. Overall body condition and general behavior were assessed daily. Tumor volume (mm) was calculated from caliper measurements as an ellipsoid: Tumor growth was expressed as relative tumor volume (RTV) which is tumor volume measured on day X divided by the tumor volume measured on the day of dosing. In the 22RV1 model, 200 nCi and 400 nCi caused long term tumor regression in all the mice (FIGS. 9A-9B). In the 22RV1 model, 100 nCi-treated group showed a mixed response including delayed tumor growth, tumor suppression and regression. In the C4-2 model, the 100 nCi-treated group caused long-term tumor regression in all the mice, while the 50 nCi-treated group caused long-term regression in 3 out of 4 mice (FIG. 9C).

The patient-derived xenograft (PDX) efficacy study was performed with two increasing doses of [²²⁵Ac]-STEAP2 (40A3-LO14) and compared to the isotype control and untreated control cohorts. The therapeutic efficacy study was carried out using the CTG-3167 prostate cancer PDX model. For the study, 5 groups of tumor-bearing animals (n=3) were injected intravenously via the lateral tail vein. [²²⁵Ac]-STEAP2 (40A3-LO14) was dosed at 50-100 nanocuries (nCi) of activity formulated in 20 mM sodium citrate pH 5.5, 0.82% NaCl, and 0.01% Tween 80 buffer. Animals were assessed daily to monitor general health and any display of acute adverse effects to the treatment. Animals were weighed and tumor measurements were taken with vernier calipers 2 times per week for up 60 days. Tumor volume was evaluated by measuring perpendicular tumor diameters and the growth of tumors in each experimental group was expressed as the mean tumor volume (mm³)±SEM of the number of animals used. In the CTG-3167 model, 50 nCi and 100 nCi caused long-term tumor regression in all mice (FIG. 9D).

### Other Embodiments

While the disclosure has been described in connection with specific embodiments thereof, it will be understood that it is capable of further modifications and this application is intended to cover any variations, uses, or adaptations of the disclosure following, in general, the principles of the disclosure and including such departures from the present disclosure that come within known or customary practice within the art to which the disclosure pertains and may be applied to the essential features herein before set forth.

What is claimed is:

1. A compound comprising Formula I, or a pharmaceutically acceptable salt thereof:
A-L¹-(L²)ₙ-B Formula I
wherein
A is a chelating moiety or a metal complex thereof,
B is an antibody or antigen-binding fragment thereof,
L¹ is a bond, C═O, C═S, optionally substituted C₁-C₆ alkyl, optionally substituted C₁-C₆ heteroalkyl, optionally substituted aryl, or optionally substituted heteroaryl;
n is an integer between 1 and 5 (inclusive); and
L² each independently has the structure of Formula II:
—X¹-L³-Z¹— Formula II
wherein
X¹ is —C(O)NR¹—*, —NR¹C(O)—*, —C(S)NR¹—*, —NR¹C(S)—*, —OC(O)NR¹—*, —NR¹C(O)O—*, —NR¹C(O)NR¹—, —CH₂-Ph-C(O)NR¹—*, —NR¹C(O)-Ph-CH₂—*, —CH₂-Ph-NH—C(S)NR¹—*, —NR¹C(S)—NH-Ph-CH₂—*, —O—, or —NR¹—, wherein “*” indicates the attachment point to L³, and R¹ is hydrogen, optionally substituted C₁-C₆ alkyl, optionally substituted C₁-C₆ heteroalkyl, optionally substituted aryl, or optionally substituted heteroaryl;
L³ is optionally substituted C₁-C₅₀alkyl or optionally substituted C₁-C₅₀ heteroalkyl; and
Z¹ is —CH₂—#, —C(O)—#, —C(S)—#, —OC(O)—#, —C(O)O—#, —NR²C(O)—#, —C(O)NR²—#, or —NR²—#, wherein “#” indicates the attachment point to B, and R² is hydrogen, optionally substituted C₁-C₆ alkyl, optionally substituted C₁-C₆ heteroalkyl, optionally substituted aryl, or optionally substituted heteroaryl,
wherein the antibody or antigen-binding fragment thereof binds to six-transmembrane epithelial antigen of prostate-2 (STEAP2) and comprises:
a) HCDR1 comprising the sequence of SEQ ID NO:1, HCDR2 comprising the sequence of SEQ ID NO:2 and HCDR3 comprising the sequence of SEQ ID NO:3 and LCDR1 comprising the sequence of SEQ ID NO:6, LCDR2 comprising the sequence of SEQ ID NO:7 and LCDR3 comprising the sequence of SEQ ID NO:8;
(b) HCDR1 comprising the sequence of SEQ ID NO:11, HCDR2 comprising the sequence of SEQ ID NO:12 and HCDR3 comprising the sequence of SEQ ID NO:13 and LCDR1 comprising the sequence of SEQ ID NO:16, LCDR2 comprising the sequence of SEQ ID NO:17 and LCDR3 comprising the sequence of SEQ ID NO:18;
(c) HCDR1 comprising the sequence of SEQ ID NO:21, HCDR2 comprising the sequence of SEQ ID NO:22 and HCDR3 comprising the sequence of SEQ ID NO:23 and LCDR1 comprising the sequence of SEQ ID NO:25, LCDR2 comprising the sequence of SEQ ID NO:26 and LCDR3 comprising the sequence of SEQ ID NO:27; or
(d) HCDR1 comprising the sequence of SEQ ID NO:31, HCDR2 comprising the sequence of SEQ ID NO:32 and HCDR3 comprising the sequence of SEQ ID NO:33 and LCDR1 comprising the sequence of SEQ ID NO:35, LCDR2 comprising the sequence of SEQ ID NO:36 and LCDR3 comprising the sequence of SEQ ID NO:37,
or a functional variant of an antibody or antigen-binding fragment of any one of (a)-(d).

2. The compound or pharmaceutically acceptable salt thereof of claim 1, wherein said chelating moiety is selected from the group consisting of DOTA (1,4,7,10-tetraazacyclododecane-1,4,7,10-tetraacetic acid), DOTMA (1R,4R,7R,10R)-α,α″,α″,α″′-tetramethyl-1,4,7,10-tetraazacyclododecane-1,4,7,10-tetraacetic acid, DOTAM (1,4,7,10-tetrakis(carbamoylmethyl)-1,4,7,10-tetraazacyclododecane), DOTPA (1,4,7,10-tetraazacyclododecane-1,4,7,10-tetra propionic acid), DO3AM-acetic acid (2-(4,7,10-tris(2-amino-2-oxoethyl)-1,4,7,10-tetraazacyclododecan-1-yl)acetic acid), DOTA-GA anhydride (2,2′,2″-(10-(2,6-dioxotetrahydro-2H-pyran-3-yl)-1,4,7,10-tetraazacyclododecane-1,4,7-triyl)triacetic acid, DOTP (1,4,7,10-tetraazacyclododecane-1,4,7,10-tetra(methylene phosphonic acid)), DOTMP (1,4,6,10-tetraazacyclodecane-1,4,7,10-tetramethylene phosphonic acid, DOTA-4AMP (1,4,7,10-tetraazacyclododecane-1,4,7,10-tetrakis(acetamido-methylenephosphonic acid), CB-TE2A (1,4,8,11-tetraazabicyclo[6.6.2]hexadecane-4,11-diacetic acid), NOTA (1,4,7-triazacyclononane-1,4,7-triacetic acid), NOTP (1,4,7-triazacyclononane-1,4,7-tri(methylene phosphonic acid), TETPA (1,4,8,11-tetraazacyclotetradecane-1,4,8,11-tetrapropionic acid), TETA (1,4,8,11-tetraazacyclotetradecane-1,4,8,11-tetra acetic acid), HEHA (1,4,7,10,13,16-hexaazacyclohexadecane-1,4,7,10,13,16-hexaacetic acid), PEPA (1,4,7,10,13-pentaazacyclopentadecane-N,N′,N″,N″′,N″″-pentaacetic acid), H₄octapa (N,N′-bis(6-carboxy-2-pyridylmethyl)-ethylenediamine-N,N′-diacetic acid), H₂dedpa (1,2-[[6-(carboxy)-pyridin-2-yl]-methylamino]ethane), H₆phospa (N,N′-(methylenephosphonate)-N,N′-[6-(methoxycarbonyl)pyridin-2-yl]-methyl-1,2-diaminoethane), TTHA (triethylenetetramine-N,N,N′,N″,N″′,N″′-hexaacetic acid), DO2P (tetraazacyclododecane dimethanephosphonic acid), HP-D03A (hydroxypropyltetraazacyclododecanetriacetic acid), EDTA (ethylenediaminetetraacetic acid), Deferoxamine, DTPA (diethylenetriaminepentaacetic acid), DTPA-BMA (diethylenetriaminepentaacetic acid-bismethylamide), and porphyrin.

3. The compound or pharmaceutically acceptable salt thereof of claim 2, wherein the compound is represented by: 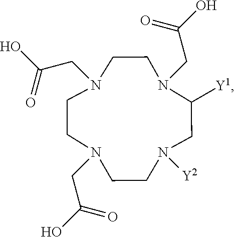
wherein Y¹ is —CH₂OCH₂(L²)ₙ-B, C═O(L²)ₙ-B, or C=S(L²)ₙ-B and Y² is —CH₂CO₂H; or
wherein Y¹ is H and Y² is L¹-(L²)ₙ-B.

4. The compound or pharmaceutically acceptable salt thereof of any one of claims 1-3, wherein L¹ is 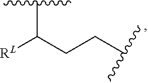
wherein R^{L} is hydrogen or —CO₂H.

5. The compound or pharmaceutically acceptable salt thereof of any one of claims 1-4,
wherein the metal complex comprises a metal selected from the group consisting of Bi, Pb, Y, Mn, Cr, Fe, Co, Zn, Ni, Tc, In, Ga, Cu, Re, a lanthanide, and an actinide; or
wherein the metal complex comprises a radionuclide selected from the group consisting of ⁴⁴Sc, ⁴⁷Sc, ⁵⁵Co, ⁶⁰Cu, ⁶¹Cu, ⁶²Cu, ⁶⁴Cu, ⁶⁷Cu, ⁶⁶Ga ⁶⁷Ga, ⁶⁸Ga, ⁸²Rb, ⁸⁶Y, ⁸⁷Y⁸⁹Zr, ⁹⁰Y, ⁹⁷Ru, ⁹⁹Tc, ^{99m}Tc, ¹⁰⁵Rh, ¹⁰⁹Pd, ¹¹n, ^{117m}Sn, ¹⁴⁹Pm, ¹⁴⁹Tb, ¹⁵³Sm, ¹⁶⁶Ho, ¹⁷⁷Lu, ¹⁸⁶Re, ¹⁸⁸Re, ¹⁹⁸Au, ¹⁹⁹Au, ²⁰¹Tl, ²⁰³Pb, ²¹¹At, ²¹²Pb, ²¹²Bi, ²¹³Bi, ²²³Ra, ²²⁵Ac, ²²⁷Th, and ²²⁹Th.

6. The compound or pharmaceutically acceptable salt thereof of any one of claims 3-5, wherein Y¹ is H.

7. The compound or pharmaceutically acceptable salt thereof of any one of claims 1-6 wherein X¹ is —C(O)NR¹—*or —NR¹C(O)—*, “*” indicating the attachment point to L³, and R¹ is H.

8. The compound or pharmaceutically acceptable salt thereof of any one of claims 1-7, wherein Z¹ is —CH₂—.

9. The compound or pharmaceutically acceptable salt thereof of any one of claims 1-8, wherein n is 1, and L³ comprises (CH₂CH₂O)₂₋₂₀.

10. The compound or pharmaceutically acceptable salt thereof of any one of claims 1-8, wherein n is 1, and L³ is (CH₂CH₂O)ₘ(CH₂)_{w}, wherein m and w are each independently an integer between 0 and 10 (inclusive), and at least one of m and w is not 0.

11. The compound or pharmaceutically acceptable salt thereof of claim 1, wherein the compound comprises: 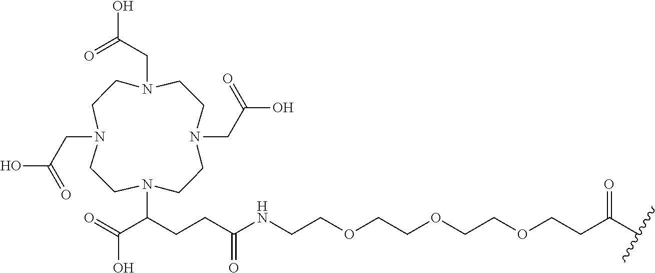 [SMILES: *C(=O)CCOCCOCCOCCNC(=O)CCC(C(=O)O)N1CCN(CC(=O)O)CCN(CC(=O)O)CCN(CC(=O)O)CC1]
or a metal complex thereof, or the compound comprises 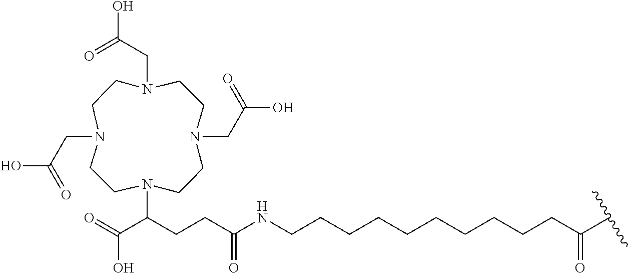 [SMILES: *C(=O)CCCCCCCCCCNC(=O)CCC(C(=O)O)N1CCN(CC(=O)O)CCN(CC(=O)O)CCN(CC(=O)O)CC1]
or a metal complex thereof.

12. The compound or pharmaceutically acceptable salt thereof of claim 1, wherein the compound comprises: 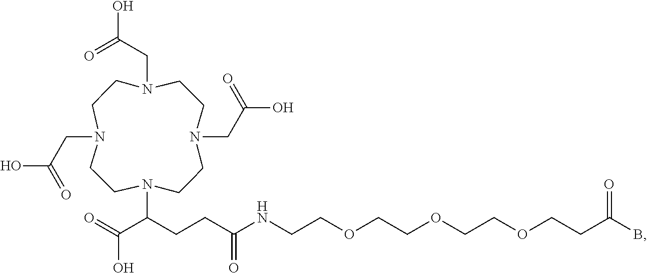
or a metal complex thereof.

13. The compound or pharmaceutically acceptable salt thereof of any one of claims 1-12, wherein A is a metal complex of a chelating moiety, and the metal complex comprises a radionuclide.

14. The compound or pharmaceutically acceptable salt thereof of claim 13, wherein the radionuclide is ⁶⁸Ga, ¹¹¹In ¹⁷⁷Lu, or ²²⁵Ac.

15. The compound or pharmaceutically acceptable salt thereof of claim 13, wherein the radionuclide is an alpha emitter selected from the group consisting of Astatine-211 (²¹¹At), Bismuth-212 (²¹²Bi), Bismuth-213 (²¹³Bi), Actinium-225 (²²⁵Ac), Radium-223 (²²³Ra), Lead-212 (²¹²Pb), Thorium-227 (²²⁷Th), and Terbium-149 (¹⁴⁹Tb), or a progeny thereof.

16. The compound or pharmaceutically acceptable salt thereof of claim 15, wherein the alpha emitter is ²²⁵Ac or a progeny thereof.

17. The compound or pharmaceutically acceptable salt thereof of claim 13, wherein the radionuclide is ²²⁵Ac.

18. The compound or pharmaceutically acceptable salt thereof of any one of the preceding claims, wherein the antibody or antigen-binding fragment thereof comprises HCDR1 comprising the sequence of SEQ ID NO:1, HCDR2 comprising the sequence of SEQ ID NO:2 and HCDR3 comprising the sequence of SEQ ID NO:3 and LCDR1 comprising the sequence of SEQ ID NO:6, LCDR2 comprising the sequence of SEQ ID NO:7 and LCDR3 comprising the sequence of SEQ ID NO:8.

19. The compound or pharmaceutically acceptable salt thereof of any one of the preceding claims, wherein the antibody or antigen-binding fragment thereof comprises a VH domain having at least 80%, 85%, 90% or 95% sequence identity to the amino acid sequence of SEQ ID NO:4 and a VL domain having at least 80%, 85%, 90% or 95% sequence identity to the amino acid sequence of SEQ ID NO:9.

20. The compound or pharmaceutically acceptable salt thereof of any one of the preceding claims, wherein the antibody or antigen-binding fragment thereof comprises a VH domain having at least 95% sequence identity to the amino acid sequence of SEQ ID NO:4 and a VL domain having at least 95% sequence identity to the amino acid sequence of SEQ ID NO:9.

21. The compound or pharmaceutically acceptable salt thereof of claim 20, wherein the VH domain comprises the amino acid sequence of SEQ ID NO:4 and the VL domain comprises the amino acid sequence of SEQ ID NO:9.

22. The compound or pharmaceutically acceptable salt thereof of any one of the preceding claims, wherein the antibody or antigen-binding fragment thereof comprises a heavy chain having at least 80%, 85%, 90% or 95% sequence identity to the amino acid sequence of SEQ ID NO:5 and a light chain having at least 80%, 85%, 90% or 95% sequence identity to the amino acid sequence of SEQ ID NO:10.

23. The compound or pharmaceutically acceptable salt thereof of any one of the preceding claims, wherein the antibody or antigen-binding fragment thereof comprises a heavy chain having at least 95% sequence identity to the amino acid sequence of SEQ ID NO:5 and a light chain having at least 95% sequence identity to the amino acid sequence of SEQ ID NO: 10.

24. The compound or pharmaceutically acceptable salt thereof of claim 23, wherein the heavy chain comprises the amino acid sequence of SEQ ID NO:5 and the light chain comprises the amino acid sequence of SEQ ID NO:10.

25. The compound or pharmaceutically acceptable salt thereof of any one of the preceding claims, wherein the antibody or antigen-binding fragment thereof binds to a STEAP2 (preferably a human STEAP2) with a binding affinity of between about 0.1 nM to about 40 nM, between about 0.5 nM to about 30 nM, between about 1 nM to about 20 nM, or between about 1 nM to about 10 nM.

26. The compound or pharmaceutically acceptable salt thereof of any one of the preceding claims, wherein the compound comprises: 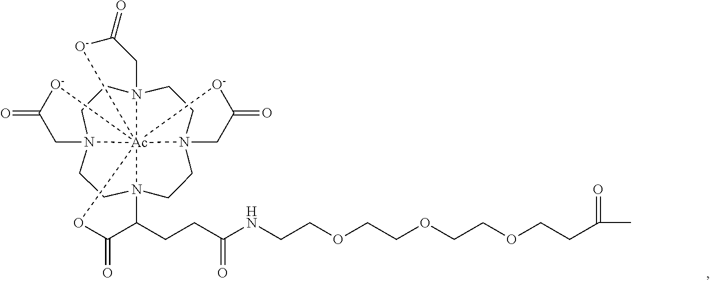
Wherein [symbol] is an antibody or antigen-binding fragment thereof as defined in anyone of the preceding claims.

27. The compound or pharmaceutically acceptable salt thereof of claim 26, wherein the antibody or antigen-binding fragment thereof is linked to A-L¹-(L²)ₙ- via the side-chain amino group of a lysine residue.

28. A compound comprising Formula I, or a pharmaceutically acceptable salt thereof:
A-L¹-(L²)ₙ-B Formula I
wherein
A is a chelating moiety or a metal complex thereof,
B is an antibody or antigen-binding fragment thereof,
L¹ is a bond, C═O, C═S, optionally substituted C₁-C₆ alkyl, optionally substituted C₁-C₆ heteroalkyl, optionally substituted aryl, or optionally substituted heteroaryl;
n is an integer between 1 and 5 (inclusive); and
L² each independently has the structure of Formula II:
—X¹-L³-Z¹— Formula II
wherein
X¹ is —C(O)NR¹—*, —NR¹C(O)—*, —C(S)NR¹—*, —NR¹C(S)—*, —OC(O)NR¹—*, —NR¹C(O)O—*, —NR¹C(O)NR¹—, —CH₂-Ph-C(O)NR¹—*, —NR¹C(O)-Ph-CH₂—*, —CH₂-Ph-NH—C(S)NR¹—*, —NR¹C(S)—NH-Ph-CH₂—*, —O—, or —NR¹—, wherein “*” indicates the attachment point to L³, and R¹ is hydrogen, optionally substituted C₁-C₆ alkyl, optionally substituted C₁-C₆ heteroalkyl, optionally substituted aryl, or optionally substituted heteroaryl;
L³ is optionally substituted C₁-C₅₀alkyl or optionally substituted C₁-C₅₀ heteroalkyl; and
Z¹ is —CH₂—#, —C(O)—#, —C(S)—#, —OC(O)—#, —C(O)O—#, —NR²C(O)—#, —C(O)NR²—#, or —NR²—#, wherein “#” indicates the attachment point to B, and R² is hydrogen, optionally substituted C₁-C₆ alkyl, optionally substituted C₁-C₆ heteroalkyl, optionally substituted aryl, or optionally substituted heteroaryl,
wherein the antibody or antigen-binding fragment thereof binds to six-transmembrane epithelial antigen of prostate-2 (STEAP2) and competes for binding with an antibody or antigen-binding fragment that comprises
a) HCDR1 comprising the sequence of SEQ ID NO:1, HCDR2 comprising the sequence of SEQ ID NO:2 and HCDR3 comprising the sequence of SEQ ID NO:3 and LCDR1 comprising the sequence of SEQ ID NO:6, LCDR2 comprising the sequence of SEQ ID NO:7 and LCDR3 comprising the sequence of SEQ ID NO:8;
(b) HCDR1 comprising the sequence of SEQ ID NO:11, HCDR2 comprising the sequence of SEQ ID NO:12 and HCDR3 comprising the sequence of SEQ ID NO:13 and LCDR1 comprising the sequence of SEQ ID NO:16, LCDR2 comprising the sequence of SEQ ID NO:17 and LCDR3 comprising the sequence of SEQ ID NO:18;
(c) HCDR1 comprising the sequence of SEQ ID NO:21, HCDR2 comprising the sequence of SEQ ID NO:22 and HCDR3 comprising the sequence of SEQ ID NO:23 and LCDR1 comprising the sequence of SEQ ID NO:25, LCDR2 comprising the sequence of SEQ ID NO:26 and LCDR3 comprising the sequence of SEQ ID NO:27; or
(d) HCDR1 comprising the sequence of SEQ ID NO:31, HCDR2 comprising the sequence of SEQ ID NO:32 and HCDR3 comprising the sequence of SEQ ID NO:33 and LCDR1 comprising the sequence of SEQ ID NO:35, LCDR2 comprising the sequence of SEQ ID NO:36 and LCDR3 comprising the sequence of SEQ ID NO:37.

29. A pharmaceutical composition comprising a compound or pharmaceutically acceptable salt thereof of any one of claims 1-28 and a pharmaceutically acceptable carrier, diluent, or excipient.

30. A method of treating a cancer in a subject, said method comprising administering to the subject a therapeutically effective amount of a compound or pharmaceutically acceptable salt thereof of any one of claims 1-28 or the pharmaceutical composition of claim 29.

31. The method of claim 30, wherein the cancer is a solid tumor cancer selected from the group consisting of prostate cancer, bladder cancer, breast cancer, colorectal carcinoma, and gastric cancer.

32. The method of claim 31, wherein the cancer is prostate cancer.

33. The method of any one of claims 30-32, further comprising administering to the subject an antiproliferative agent, a radiation sensitizer, or an immunomodulatory agent.

34. A compound or pharmaceutically acceptable salt thereof of any one of claims 1-28, or the pharmaceutical composition of claim 29, for use in a method of treating cancer.

35. Use of a compound or pharmaceutically acceptable salt thereof of any one of claims 1-28, or the pharmaceutical composition of claim 29, in the manufacture of a medicament for the treatment of cancer.

36. The use of claim 35, wherein the cancer is a solid tumor cancer selected from the group consisting of prostate cancer, bladder cancer, breast cancer, colorectal carcinoma, and gastric cancer.

37. The use of claim 36, wherein the cancer is prostate cancer.

38. The use of any one of claims 35-37, wherein the treatment further comprises an antiproliferative agent, a radiation sensitizer, or an immunomodulatory agent.


SEQUENCE LISTING (40 sequences)

SEQ ID NO: 1 - protein, 7 aa, synthetic construct
RNSAVWN

SEQ ID NO: 2 - protein, 18 aa, synthetic construct
RTYYRSKWYNDYAPSVKS

SEQ ID NO: 3 - protein, 13 aa, synthetic construct
GLRQNQFYYYMDV

SEQ ID NO: 4 - protein, 125 aa, synthetic construct
QVQLQQSGPGLVKPSQTLSLTCAISGDSVSRNSAVWNWIRQSPSRGLEWLGRTYYRSKWYNDYAPSVKSRITINPDTSKNQFSLQLNSVTPEDTAVYYCARGLRQNQFYYYMDVWGKGTTVTVSS

SEQ ID NO: 5 - protein, 455 aa, synthetic construct
QVQLQQSGPGLVKPSQTLSLTCAISGDSVSRNSAVWNWIRQSPSRGLEWLGRTYYRSKWYNDYAPSVKSRITINPDTSKNQFSLQLNSVTPEDTAVYYCARGLRQNQFYYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 11 aa, synthetic construct
RASQSVASNLA

SEQ ID NO: 7 - protein, 7 aa, synthetic construct
GASTRAT

SEQ ID NO: 8 - protein, 9 aa, synthetic construct
QQYNNWPFT

SEQ ID NO: 9 - protein, 107 aa, synthetic construct
EIVMTQSPATLSVSPGERATLSCRASQSVASNLAWYQQKPGQAPRLLIYGASTRATGIPARFSGSGSGTEFTLTISSLQSEDFAVYYCQQYNNWPFTFGPGTKVDIK

SEQ ID NO: 10 - protein, 214 aa, synthetic construct
EIVMTQSPATLSVSPGERATLSCRASQSVASNLAWYQQKPGQAPRLLIYGASTRATGIPARFSGSGSGTEFTLTISSLQSEDFAVYYCQQYNNWPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 7 aa, synthetic construct
RNSAVWN

SEQ ID NO: 12 - protein, 18 aa, synthetic construct
RTYYRSKWYNDYAPSVKS

SEQ ID NO: 13 - protein, 13 aa, synthetic construct
GLLQNQFYYYMDV

SEQ ID NO: 14 - protein, 125 aa, synthetic construct
QVQLQQSGPGLVKPSQTLSLTCAISGDSVSRNSAVWNWIRQSPSRGLEWLGRTYYRSKWYNDYAPSVKSRITINPDTSKNQFSLQLNSVTPEDTAVYYCARGLLQNQFYYYMDVWGKGTTVTVSS

SEQ ID NO: 15 - protein, 455 aa, synthetic construct
QVQLQQSGPGLVKPSQTLSLTCAISGDSVSRNSAVWNWIRQSPSRGLEWLGRTYYRSKWYNDYAPSVKSRITINPDTSKNQFSLQLNSVTPEDTAVYYCARGLLQNQFYYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 11 aa, synthetic construct
RASQSVASNLA

SEQ ID NO: 17 - protein, 7 aa, synthetic construct
GASTRAT

SEQ ID NO: 18 - protein, 9 aa, synthetic construct
QQYNNWPFT

SEQ ID NO: 19 - protein, 107 aa, synthetic construct; identical to SEQ ID NO: 9

SEQ ID NO: 20 - protein, 214 aa, synthetic construct; identical to SEQ ID NO: 10

SEQ ID NO: 21 - protein, 7 aa, synthetic construct
RNSAVWN

SEQ ID NO: 22 - protein, 18 aa, synthetic construct
RTYYRSKWYNDYAVSVKS

SEQ ID NO: 23 - protein, 13 aa, synthetic construct
GLLQNNFYYYMDV

SEQ ID NO: 24 - protein, 125 aa, synthetic construct
QVQLQQSGPGLVKPSQTLSLTCAISGDSVSRNSAVWNWIRQSPSRGLEWLGRTYYRSKWYNDYAVSVKSRITINPDTSKNQFSLQVNSVTPEDTAVYYCARGLLQNNFYYYMDVWGKGTTVTVSS

SEQ ID NO: 25 - protein, 11 aa, synthetic construct
RASQSVNSNLA

SEQ ID NO: 26 - protein, 7 aa, synthetic construct
GASTRAT

SEQ ID NO: 27 - protein, 9 aa, synthetic construct
QQYNNWPFT

SEQ ID NO: 28 - protein, 107 aa, synthetic construct
EIVMTQSPATLSVSPGERATLSCRASQSVNSNLAWYQQKPGQAPRLLIYGASTRATGIPARFSGSGSGTEFTLTISSLQSEDFAVYYCQQYNNWPFTFGPGTKVDIK

SEQ ID NO: 29 - protein, 490 aa, Homo sapiens
MESISMMGSPKSLSETFLPNGINGIKDARKVTVGVIGSGDFAKSLTIRLIRCGYHVVIGSRNPKFASEFFPHVVDVTHHEDALTKTNIIFVAIHREHYTSLWDLRHLLVGKILIDVSNNMRINQYPESNAEYLASLFPDSLIVKGFNVVSAWALQLGPKDASRQVYICSNNIQARQQVIELARQLNFIPIDLGSLSSAREIENLPLRLFTLWRGPVVVAISLATFFFLYSFVRDVIHPYARNQQSDFYKIPIEIVNKTLPIVAITLLSLVYLAGLLAAAYQLYYGTKYRRFPPWLETWLQCRKQLGLLSFFFAMVHVAYSLCLPMRRSERYLFLNMAYQQVHANIENSWNEEEVWRIEMYISFGIMSLGLLSLLAVTSIPSVSNALNWREFSFIQSTLGYVALLISTFHVLIYGWKRAFEEEYYRFYTPPNFVLALVLPSIVILGKIILFLPCISRKLKRIKKGWEKSQFLEEGMGGTIPHVSPERVTVM

SEQ ID NO: 30 - protein, 498 aa, synthetic construct
MSHQPAVATKMPEEMDKPLISLHLVDSDSSLAKVPDEAPKVGILGSGDFARSLATRLVGSGFKVVVGSRNPKRTARLFPSAAQVTFQEEAVSSPEVIFVAVFREHYSSLCSLSDQLAGKILVDVSNPTEQEHLQHRESNAEYLASLFPTCTVVKAFNVISAWTLQAGPRDGNRQVPICGDQPEAKRAVSEMALAMGFMPVDMGSLASAWEVEAMPLRLLPAWKVPTLLALGLFVCFYAYSFVRDVIHPYARNQQSDFYKIPIEIVNKTLPCVAYVLLSLVYLPGVLAAALQLRRGTKYQRFPDWLDHWLQHRKQIGLLSFFCAALHALYSFCLPLRRSERYLFLNMAYQQVHANIENSWNEEEVWRIEIYLSLGVLALGTLSLLAVTSLPSIANSLNWREFSFVQSSLGFVALVLSTLHTLTYGWKRAFEEEYYRFYTPPNFTLTLLVPCVVILAKALFLLPCISRRLARIRRGWERESTIKFTLPTDHALAEKTSHV

SEQ ID NO: 31 - protein, 7 aa, synthetic construct
RNSAVWN

SEQ ID NO: 32 - protein, 18 aa, synthetic construct
RTYYRSKWYNDYAVSVKS

SEQ ID NO: 33 - protein, 13 aa, synthetic construct
GLLQNQFYYYMDV

SEQ ID NO: 34 - protein, 125 aa, synthetic construct
QVQLQQSGPGLVKPSQTLSLTCAISGDSVSRNSAVWNWIRQSPSRGLEWLGRTYYRSKWYNDYAVSVKSRITINPDTSKNQFSLQLNSVTPEDTAVYYCARGLLQNQFYYYMDVWGKGTTVTVSS

SEQ ID NO: 35 - protein, 11 aa, synthetic construct
RASQSVSSNLA

SEQ ID NO: 36 - protein, 7 aa, synthetic construct
GASTRAT

SEQ ID NO: 37 - protein, 9 aa, synthetic construct
QQYNNWPFT

SEQ ID NO: 38 - protein, 107 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYNNWPFTFGPGTKVDIK

SEQ ID NO: 39 - protein, 5 aa, synthetic construct
  VARIANT 3: note = Any amino acid
LPXTG

SEQ ID NO: 40 - protein, 5 aa, synthetic construct
  VARIANT 4: note = Any amino acid
LPTXG